(19) United States
(12) Patent Application Publication
(10) Pub. No.: US 2023/0348853 A1
(43) Pub. Date: Nov. 2, 2023

(54) C825 EXPRESSING IMMUNE CELLS AND DIAGNOSTIC USES THEREOF

(71) Applicant: Memorial Sloan Kettering Cancer Center, New York, NY (US)

(72) Inventors: Steven Larson, New York, NY (US); Darren Veach, New York, NY (US); Sarah Cheal, New York, NY (US); Ouathek Ouerfelli, New York, NY (US); Guangbin Yang, New York, NY (US); Simone Krebs, New York, NY (US); Megan Dacek, New York, NY (US); David Scheinberg, New York, NY (US); Nai-Kong Cheung, New York, NY (US); Brian Santich, New York, NY (US)

(21) Appl. No.: 18/003,646

(22) PCT Filed: Jun. 28, 2021
(86) PCT No.: PCT/US2021/039418

§ 371 Date: Dec. 28, 2022

Related U.S. Application Data
(60) Provisional application No. 63/045,641, filed on Jun. 29, 2020.

(51) Int. Cl. C12N 5/078; C07K 16/44; G01N 33/574; C12N 15/63; C07K 14/725

(52) U.S. Cl. CPC C12N 5/0634; C07K 16/44; G01N 33/57492; C12N 15/63; C07K 14/7051; C07K 2319/03; C07K 2317/622; G01N 2800/7028

(57) ABSTRACT

Provided herein are compositions comprising engineered immune cells that express a tumor antigen-targeted chimeric antigen receptor and an anti-DOTA C825 antigen binding fragment. The engineered immune cells of the present technology are configured to bind to DOTA haptens that may be complexed with a diagnostic radiopharmaceutical (e.g., ¹¹¹In). Also disclosed herein are methods for determining the in vivo biodistribution, viability, and expansion of the engineered immune cells described herein.

### CROSS-REFERENCE TO RELATED APPLICATIONS

This application is a U.S. National Stage Application of PCT/US2021/039418, filed Jun. 28, 2021, which claims the benefit of and priority to U.S. Provisional Patent Application No. 63/045,641, filed Jun. 29, 2020, the contents of which are incorporated by reference herein in its entirety.

### STATEMENT OF GOVERNMENT SUPPORT

This invention was made with government support under CA55349 and CA23766, awarded by the National Institutes of Health/National Cancer Institute. The government has certain rights in the invention.

### TECHNICAL FIELD

The present technology relates generally to compositions including engineered immune cells that express a tumor antigen-targeted chimeric antigen receptor and an anti-DOTA C825 antigen binding fragment. Also disclosed herein are methods for determining the in vivo biodistribution, viability, and expansion of the compositions disclosed herein.

### SEQUENCE LISTING

The instant application contains a Sequence Listing which has been submitted electronically in ASCII format and is hereby incorporated by reference in its entirety. Said ASCII copy, created on Jul. 27, 2021, is named 115872-2236_SL.txt and is 77,752 bytes in size.

### BACKGROUND

The following description of the background of the present technology is provided simply as an aid in understanding the present technology and is not admitted to describe or constitute prior art to the present technology.

Chimeric antigen receptor (CAR) T cell therapy redirects T cells to activate in the presence of, and subsequently kill, an antigen-expressing cell. This is achieved by coupling an antigen-specific single-chain variable fragment (scFv) to endogenous T cell activation signaling domains. CAR T cells enhance the ability of the immune cells to recognize and destroy individual cancer cells.

CAR T cell therapy has been especially potent for tumors of the blood and lymph nodes, i.e., leukemia and lymphoma. However, many tumors do not respond well to CAR T cell therapy, especially solid tumors of the colon, lung, and breast. D'Aloia et al., Cell Death & Disease 9, 282 (2018); Singh et al., Semin Cancer Biol. S1044-579X(19)30398-0 (2019). One reason might be that CAR-T cells do not find their way to the tumor due to a variety of resistance mechanisms, including the tumor microenvironment that deflects immune surveillance and attack. Other challenges include heterogeneously expressed tumor target antigens and impaired long-term persistence of CAR T cells at the tumor site.

Thus, there is an urgent need for methods that accurately and reliably “track” or “trace” CAR-T cells within the body of a patient.

### SUMMARY OF THE PRESENT TECHNOLOGY

Disclosed herein are methods for determining the in vivo biodistribution, viability, and expansion of engineered immune cells of any immune specificity.

Provided herein, in certain embodiments, are compositions comprising engineered immune cells that express an anti-DOTA C825 antigen binding fragment and a receptor that binds to a tumor antigen. In some embodiments, the receptor is a T cell receptor. In some embodiments, the receptor is a native receptor (e.g., a native T cell receptor). In some embodiments, the receptor is a non-native receptor (e.g., a non-native T cell receptor), for example, an engineered receptor, such as a chimeric antigen receptor (CAR). In some embodiments, the engineered immune cells comprise an anti-DOTA C825 antigen binding fragment and/or a nucleic acid encoding the anti-DOTA C825 antigen binding fragment. In some embodiments, the engineered immune cells comprise a chimeric antigen receptor and/or nucleic acid encoding the chimeric antigen receptor. In some embodiments, the nucleic acid encoding the anti-DOTA C825 antigen binding fragment is operably linked to a promoter. The promoter may be a constitutive promoter or a conditional promoter. In some embodiments, the conditional promoter is inducible by binding of the receptor (e.g., a CAR) to an antigen, such as a tumor antigen. In some embodiments, the chimeric antigen receptor comprises (i) an extracellular antigen binding domain; (ii) a transmembrane domain; and (iii) an intracellular domain. In some embodiments, the extracellular antigen binding domain binds to a tumor antigen. In some embodiments, the tumor antigen is selected from among 5T4, alpha 5β1-integrin, 707-AP, A33, AFP, ART-4, B7H4, BAGE, Bcl-2, β-catenin, BCMA, Bcr-abl, MN/C IX antibody, CA125, CA19-9, CAMEL, CAP-1, CASP-8, CD4, CD5, CD19, CD20, CD21, CD22, CD25, CDC27/m, CD33, CD37, CD45, CD52, CD56, CD80, CD123, CDK4/m, CEA, c-Met, CS-1, CT, Cyp-B, cyclin B1, DAGE, DAM, EBNA, EGFR, ErbB3, ELF2M, EMMPRIN, EpCam, ephrinB2, estrogen receptor, ETV6-AML1, FAP, ferritin, folate-binding protein, GAGE, G250, GD-2, GM2, GnT-V, gp75, gp100 (Pmel 17), HAGE, HER-2/neu, HLA-A*0201-R170I, HPV E6, HPV E7, Ki-67, HSP70-2M, HST-2, hTERT (or hTRT), iCE, IGF-1R, IL-2R, IL-5, KIAA0205, LAGE, LDLR/FUT, LRP, MAGE, MART, MART-1/melan-A, MART-2/Ski, MC1R, mesothelin, MUC, MUC16, MUM-1-B, myc, MUM-2, MUM-3, NA88-A, NYESO-1, NY-Eso-B, p53, proteinase-3, p190 minor ber-abl, Pml/RARα, PRAME, progesterone receptor, PSA, PSCA, PSM, PSMA, ras, RAGE, RU1 or RU2, RORI, SART-1 or SART-3, survivin, TEL/AML1, TGFβ, TPI/m, TRP-1, TRP-2, TRP-2/INT2, tenascin, TSTA tyrosinase, VEGF, and WT1.

In some embodiments, the extracellular antigen binding domain of the chimeric antigen receptor comprises a single chain variable fragment (scFv). In some embodiments, the extracellular antigen binding domain of the chimeric antigen receptor comprises a human scFv. In some embodiments, the extracellular antigen binding domain of the chimeric antigen receptor comprises a CD19 scFv of SEQ ID NO: 3 or SEQ ID NO: 4. In some embodiments, the extracellular antigen binding domain of the chimeric antigen receptor comprises a CD19 scFv having at least 80%, 85%, 90%, 95%, 96%, 97%, 98%, or 99% sequence identity to SEQ ID NO: 3 or SEQ ID NO: 4. In some embodiments, the extracellular antigen binding domain of the chimeric antigen receptor comprises a signal peptide that is covalently joined to the N-terminus of the extracellular antigen binding domain. In some embodiments, the transmembrane domain of the chimeric antigen receptor comprises a CD8 transmembrane domain. In some embodiments, the intracellular domain of the chimeric antigen receptor comprises one or more costimulatory domains. In some embodiments, the one or more costimulatory domains are selected from a CD28 costimulatory domain, a CD3ζ-chain, a 4-1BBL costimulatory domain, or any combination thereof. In some embodiments, the immune cell is a lymphocyte. In some embodiments, the lymphocyte is a T-cell, a B cell or a natural killer (NK) cell. In some embodiments, the T cell is a CD4⁺ T cell or a CD8⁺ T cell. In some embodiments, the immune cell is a tumor infiltrating lymphocyte. In some embodiments, the immune cell is derived from an autologous donor or an allogenic donor.

Also provided are polypeptides comprising an anti-DOTA C825 antigen binding fragment and a chimeric antigen receptor. In some embodiments, the polypeptides further comprise a self-cleaving peptide located between the anti-DOTA C825 antigen binding fragment and the chimeric antigen receptor. In some embodiments, the self-cleaving peptide is a P2A self-cleaving peptide. In some embodiments, the anti-DOTA C825 antigen binding fragment comprises a signal peptide for secretion of the anti-DOTA C825 antigen binding fragment. In some embodiments, the anti-DOTA C825 antigen binding fragment comprises the amino acid sequence of any one of SEQ ID NOs: 35-39, 41 or 42. In some embodiments, the anti-DOTA C825 antigen binding fragment is a scFv. Additionally or alternatively, in some embodiments, the chimeric antigen receptor comprises (i) an antigen binding domain; (ii) a transmembrane domain; and (iii) an intracellular domain. In some embodiments, the antigen binding domain of the chimeric antigen receptor binds to a tumor antigen. In some embodiments, the tumor antigen is selected from among BCMA, CD19, mesothelin, MUC16, PSCA, WT1, and PRAME. In some embodiments, the antigen binding domain of the chimeric antigen receptor comprises a single chain variable fragment (scFv). In some embodiments, the extracellular antigen binding domain of the chimeric antigen receptor comprises a CD19 scFv of SEQ ID NO: 3 or SEQ ID NO: 4. In some embodiments, the extracellular antigen binding domain of the chimeric antigen receptor comprises a CD19 scFv having at least 80%, 85%, 90%, 95%, 96%, 97%, 98%, or 99% sequence identity to SEQ ID NO: 3 or SEQ ID NO: 4. In some embodiments, the transmembrane domain of the chimeric antigen receptor comprises a CD8 transmembrane domain. In some embodiments, the intracellular domain of the chimeric antigen receptor comprises one or more costimulatory domains. In some embodiments, the one or more costimulatory domains of the chimeric antigen receptor are selected from a CD28 costimulatory domain, a CD3ζ-chain, a 4-1BBL costimulatory domain, or any combination thereof.

Also provided are nucleic acids encoding any of the polypeptides disclosed herein. In some embodiments, the nucleic acid encoding the polypeptide is operably linked to a promoter. The promoter may be a constitutive promoter or a conditional promoter. In some embodiments, the conditional promoter is inducible by the CAR binding to an antigen. Also provided are vectors comprising any of the nucleic acids disclosed herein. In some embodiments, the vector is a viral vector or a plasmid. In some embodiments, the vector is a retroviral vector.

Also provided are host cells comprising a polypeptide, a nucleic acid, or a vector disclosed herein.

In another aspect, the present disclosure provides a complex comprising any of the engineered immune cells described herein and a DOTA hapten, wherein the engineered immune cell is configured to bind to the DOTA hapten and a tumor antigen. Exemplary DOTA haptens include, but are not limited to, benzyl-DOTA, NH₂-benzyl (Bn) DOTA, DOTA-desferrioxamine, DOTA-Phe-Lys(HSG)-D-Tyr-Lys(HSG)-NH₂, Ac-Lys(HSG)D-Tyr-Lys(HSG)-Lys(Tscg-Cys)-NH₂, DOTA-D-Asp-D-Lys(HSG)-D-Asp-D-Lys(HSG)-NH₂; DOTA-D-Glu-D-Lys(HSG)-D-Glu-D-Lys(HSG)-NH₂, DOTA-D-Tyr-D-Lys(HSG)-D-Glu-D-Lys(HSG)-NH₂, DOTA-D-Ala-D-Lys(HSG)-D-Glu-D-Lys(HSG)-NH₂, DOTA-D-Phe-D-Lys(HSG)-D-Tyr-D-Lys(HSG)-NH₂, Ac-D-Phe-D-Lys(DOTA)-D-Tyr-D-Lys(DOTA)-NH₂, Ac-D-Phe-D-Lys(DTPA)-D-Tyr-D-Lys(DTPA)-NH₂, Ac-D-Phe-D-Lys(Bz-DTPA)-D-Tyr-D-Lys(Bz-DTPA)-NH₂, Ac-D-Lys(HSG)-D-Tyr-D-Lys(HSG)-D-Lys(Tscg-Cys)-NH₂, DOTA-D-Phe-D-Lys(HSG)-D-Tyr-D-Lys(HSG)-D-Lys(Tscg-Cys)-NH₂, (Tscg-Cys)-D-Phe-D-Lys(HSG)-D-Tyr-D-Lys(HSG)-D-Lys(DOTA)-NH₂, Tscg-D-Cys-D-Glu-D-Lys(HSG)-D-Glu-D-Lys(HSG)-NH₂, (Tscg-Cys)-D-Glu-D-Lys(HSG)-D-Glu-D-Lys(HSG)-NH₂, Ac-D-Cys-D-Lys(DOTA)-D-Tyr-D-Ala-D-Lys(DOTA)-D-Cys-NH₂, Ac-D-Cys-D-Lys(DTPA)-D-Tyr-D-Lys(DTPA)-NH₂, Ac-D-Lys(DTPA)-D-Tyr-D-Lys(DTPA)-D-Lys(Tscg-Cys)-NH₂, Ac-D-Lys(DOTA)-D-Tyr-D-Lys(DOTA)-D-Lys(Tscg-Cys)-NH₂, DOTA-RGD, DOTA-PEG-E(c(RGDyK))₂, DOTA-8-AOC-BBN, DOTA-PESIN, p-NO2-benzyl-DOTA, DOTA-biotin-sarcosine (DOTA-biotin), 1,4,7,10-tetraazacyclododecane-1,4,7,10-tetraacetic acid mono (N-hydroxysuccinimide ester) (DOTA-NHS), and DOTATyrLysDOTA. In certain embodiments of the complexes of the present technology, the DOTA hapten has the structure of Formula II

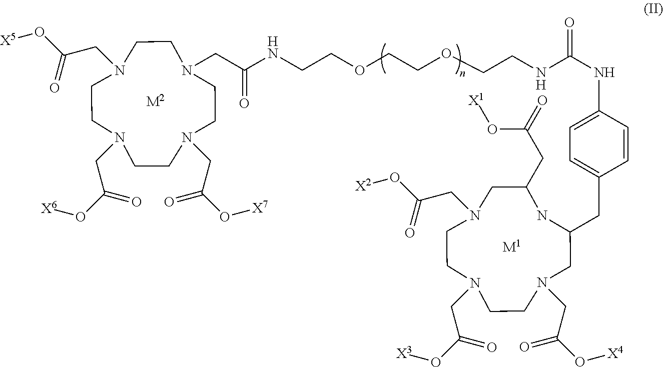

or a pharmaceutically acceptable salt thereof, wherein
M¹ is ¹⁷⁵Lu³⁺, ⁴⁵Sc³⁺, ⁶⁹Ga³⁺, ⁷¹Ga³⁺, ⁸⁹Y³⁺, ¹¹³In³⁺, ¹¹⁵In³⁺, ¹³⁹La³⁺, ¹³⁶Ce³⁺, 138Ce³⁺, ¹⁴⁰Ce³⁺, ¹⁴²Ce³⁺, ¹⁵¹Eu³⁺, ¹⁵³Eu³⁺, ¹⁵⁹Tb³⁺, ¹⁵⁴Gd³⁺, ¹⁵⁵Gd³⁺, ¹⁵⁶Gd³⁺, ¹⁵⁷Gd³⁺, ¹⁵⁸Gd³⁺, or ¹⁶⁰Gd³⁺.
M² is a radionuclide cation;
X¹, X², X³, and X⁴ are each independently a lone pair of electrons (i.e., providing an oxygen anion) or H;
X⁵, X⁶, and X⁷ are each independently a lone pair of electrons (i.e., providing an oxygen anion) or H; and
   n is 1, 2, 3, 4, 5, 6, 7, 8, 9, 10, 11, 12, 13, 14, 15, 16, 17, 18, 19, 20, 21, or 22.

In any and all embodiments of the complexes disclosed herein, M² comprises ¹¹¹In, ⁶⁷Ga ⁵¹Cr, ⁵⁸Co, ^{99m}Tc, ^{103m}Rh, ^{195m}Pt, ¹¹⁹Sb, ¹⁶¹Ho, ^{189m}Os, ¹⁹²Ir, ²⁰¹Tl, ²⁰³Pb, ⁸⁹Zr, ⁶⁸Ga, or ⁶⁴Cu.

In one aspect, the present disclosure provides a method for detecting tumors (e.g., solid or liquid tumors) in a subject in need thereof comprising (a) administering to the subject an effective amount of any complex of the present technology, wherein the complex is configured to localize to a tumor expressing the tumor antigen recognized by the engineered immune cell of the complex; and (b) detecting the presence of tumors in the subject by detecting radioactive levels emitted by the complex that are higher than a reference value. Also disclosed are methods for detecting tumors (e.g., solid or liquid tumors) in a subject in need thereof comprising (a) administering to the subject an effective amount of any engineered immune cell described herein, wherein the engineered immune cell is configured to localize to a tumor expressing the tumor antigen recognized by the engineered immune cell; (b) administering to the subject an effective amount of a radiolabeled-DOTA hapten, wherein the radiolabeled-DOTA hapten is configured to bind to the anti-DOTA C825 antigen binding fragment expressed by the engineered immune cell; and (c) detecting the presence of tumors in the subject by detecting radioactive levels emitted by the radiolabeled-DOTA hapten that are higher than a reference value. In some embodiments of the methods disclosed herein, the radioactive levels emitted by the complex or the radiolabeled-DOTA hapten are detected using positron emission tomography or single photon emission computed tomography.

Additionally or alternatively, in some embodiments, the radioactive levels emitted by the complex or the radiolabeled-DOTA hapten are detected between 4 to 24 hours after the complex or the radiolabeled-DOTA hapten is administered. In certain embodiments, the radioactive levels emitted by the complex or the radiolabeled-DOTA hapten are expressed as the percentage injected dose per gram tissue (0% ID/g). Additionally or alternatively, in some embodiments, the ratio of radioactive levels between a tumor and normal tissue is about 2:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1 or 100:1. In some embodiments, the subject is human.

In any of the preceding embodiments of the methods disclosed herein, the complex, the engineered immune cell, or the radiolabeled-DOTA hapten is administered intravenously, intratumorally, intramuscularly, intraarterially, intrathecally, intracapsularly, intraorbitally, intradermally, intraperitoneally, transtracheally, subcutaneously, intracerebroventricularly, orally or intranasally. Additionally or alternatively, in some embodiments, the complex, the engineered immune cell, or the radiolabeled-DOTA hapten is administered into the cerebral spinal fluid or blood of the subject.

In one aspect, the present disclosure provides a method for monitoring biodistribution of engineered immune cells in a subject comprising: (a) administering to the subject an effective amount of any engineered immune cell disclosed herein, wherein the engineered immune cell is configured to localize to a tissue expressing the target antigen recognized by the engineered immune cell; (b) administering to the subject an effective amount of a radiolabeled-DOTA hapten, wherein the radiolabeled-DOTA hapten is configured to bind to the anti-DOTA C825 antigen binding fragment expressed by the engineered immune cell; and (c) determining the biodistribution of engineered immune cells in the subject by detecting radioactive levels emitted by the radiolabeled-DOTA hapten that are higher than a reference value. In another aspect, the present disclosure provides a method for monitoring biodistribution of engineered immune cells in a subject comprising: (a) administering to the subject an effective amount of a complex comprising any engineered immune cell of the present technology and a radiolabeled DOTA hapten, wherein the complex is configured to localize to a tissue expressing the target antigen recognized by the engineered immune cell; and (b) determining the biodistribution of engineered immune cells in the subject by detecting radioactive levels emitted by the radiolabeled-DOTA hapten that are higher than a reference value.

In yet another aspect, the present disclosure provides a method for monitoring viability of engineered immune cells in a subject comprising: (a) administering to the subject an effective amount of any engineered immune cell disclosed herein, wherein the engineered immune cell is configured to localize to a tissue expressing the target antigen recognized by the engineered immune cell; (b) administering to the subject an effective amount of a radiolabeled-DOTA hapten, wherein the radiolabeled-DOTA hapten is configured to bind to the anti-DOTA C825 antigen binding fragment expressed by the engineered immune cell; (c) detecting radioactive levels emitted by the radiolabeled-DOTA hapten that are higher than a reference value at a first time point; (d) detecting radioactive levels emitted by the radiolabeled-DOTA hapten that are higher than a reference value at a second time point; and (e) determining that the engineered immune cells in the subject are viable when the radioactive levels emitted by the radiolabeled-DOTA hapten at the second time point are comparable to that observed at the first time point. In some embodiments, the method further comprises administering to the subject a second effective amount of the radiolabeled-DOTA hapten prior to step (d). Also disclosed herein is a method for monitoring viability of engineered immune cells in a subject comprising: (a) administering to the subject an effective amount of a complex comprising any engineered immune cell described herein and a radiolabeled DOTA hapten, wherein the complex is configured to localize to a tissue expressing the target antigen recognized by the engineered immune cell; (b) detecting radioactive levels emitted by the radiolabeled-DOTA hapten that are higher than a reference value at a first time point; (c) detecting radioactive levels emitted by the radiolabeled-DOTA hapten that are higher than a reference value at a second time point; and (d) determining that the engineered immune cells in the subject are viable when the radioactive levels emitted by the radiolabeled-DOTA hapten at the second time point are comparable to that observed at the first time point.

In yet another aspect, the present disclosure provides a method for monitoring expansion of engineered immune cells in a subject comprising: (a) administering to the subject an effective amount of any engineered immune cell described herein, wherein the engineered immune cell is configured to localize to a tissue expressing the target antigen recognized by the engineered immune cell; (b) administering to the subject a first effective amount of a radiolabeled-DOTA hapten, wherein the radiolabeled-DOTA hapten is configured to bind to the anti-DOTA C825 antigen binding fragment expressed by the engineered immune cell; (c) detecting radioactive levels emitted by the radiolabeled-DOTA hapten that are higher than a reference value at a first time point; (d) administering to the subject a second effective amount of the radiolabeled-DOTA hapten after step (c); (e) detecting radioactive levels emitted by the radiolabeled-DOTA hapten that are higher than a reference value at a second time point; and (f) determining that the engineered immune cells in the subject have expanded when the radioactive levels emitted by the radiolabeled-DOTA hapten at the second time point are higher relative to that observed at the first time point.

In any and all embodiments of the methods disclosed herein, the radioactive levels emitted by the complex or the radiolabeled-DOTA hapten are detected using positron emission tomography or single photon emission computed tomography. Additionally or alternatively, in any of the preceding embodiments of the methods disclosed herein, the engineered immune cell, the radiolabeled-DOTA hapten, or the complex is administered intravenously, intraperitoneally, subcutaneously, intramuscularly, or intratumorally.

In any and all embodiments of the methods disclosed herein, the subject is suffering from or is at risk for a cancer or tumor. In some embodiments, the cancer or tumor is a carcinoma, sarcoma, a melanoma, or a hematopoietic cancer. In some embodiments, the cancer or tumor is selected from among adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof. In some embodiments, the subject is human.

Also disclosed herein are kits containing components suitable for diagnosing or monitoring the progress of cancer in a patient. In certain embodiments, the kits comprise at least one engineered immune cell of the present technology, and instructions for use. In some embodiments, the kits comprise a DOTA hapten (e.g., any of the DOTA haptens disclosed herein), at least one engineered immune cell of the present technology, and instructions for use.

### BRIEF DESCRIPTION OF THE DRAWINGS

FIG. 1 shows the structures of exemplary DOTA haptens: Benzyl-DOTA and Proteus-DOTA(Pr).

FIGS. 2A-2C show radiohapten capture imaging results of C825-CD19 CAR-T cells with membrane expression of C825 (a picomolar binding affinity, hapten capture scFv antibody). FIG. 2A shows in vitro saturation binding curve using [¹¹¹In]Pr-DOTA binding to quantify C825 expression in C825-CD19 CAR-T cells. FIG. 2B shows a NSG mouse with a s.c. Raji GFP-fluc tumor in the right shoulder. Ten days after i.v. CAR-T cell injection (2.5×10⁶), the mouse was injected with ¹¹¹In-radiohapten for in vivo tracking of CAR T cells (either: CD19 CAR+C825, or control CD19 CAR only). FIG. 2C shows SPECT/CT images collected 18 h after injection of ¹¹¹In-radiohapten. Shown are MIP images for animals treated with C825+CD19 CAR T cells, or control CD19 CAR T cells only.

FIGS. 3A-3C show three different strategies to virally transduce primary human T cells with both C825 and CD19-CAR. FIG. 3A show transduction with two single constructs, one encoding C825 with a GFP reporter (top) and one encoding the CD19 CAR (bottom).

FIG. 3B shows a bicistronic construct encoding C825 with a transmembrane domain and GFP reporter and CD19 CAR, separated by P2A cleavage site. FIG. 3C shows a bicistronic construct encoding C825 with a Thy1 GPI linkage and His tag reporter and CD19 CAR, separated by a P2A cleavage site. Representative flow plots of transduction of primary human T cells are shown on the right.

FIG. 4A shows schematic structures of retroviral vectors SFG-Thor, SFG-19BBz (CAR) and SFG-C825. FIG. 4B shows that there is no difference between SFG-Thor T cells and SFG-19BBz (CAR) T cells with respect to killing CD19(+) Raji tumor cells as measured by in vitro 4 h cytotoxicity assays. SFG-C825 and non-transduced (NT) T cells are included as negative controls. (n=3-4 donors). FIG. 4C shows in vitro binding of [¹¹¹In]InPr at 1 h. This representative data set demonstrates the specific binding of the radiolabeled DOTA probe to C825-expressing T cells, whereas no significant uptake was observed in SFG-19BBz (CAR) and NT T cells. (All experiments were performed in triplicate at 37° C.). Data are mean±SD. FIG. 4D shows in vitro binding kinetics of [¹¹¹In]InPr to SFG-Thor T cells (n=3 independent assays; representative example shown). FIG. 4E shows an exemplary scheme of in vivo study for assessing T cell targeting to tumor cells. ⁶⁸Ga-NODAGA-Pr (100mCu, 700 pmol) was used as the radiotracer and administered 10 days after T cell administration (1×10⁶). FIG. 4F shows exemplary Maximum intensity projection (MIP) images at 1 h post-injection (p.i.) of ⁶⁸Ga-NODAGA-Pr depicting homing and accumulation of SFG-Thor T cells at the tumor (right shoulder, red arrow). No uptake above background at the tumor site is noted following SFG-19BBz (CAR) T cell administration (blue arrow). FIG. 4G shows mean uptake in tumors and tumor-to-normal-tissue-ratios (TNR) (SFG-Thor: n=4; SFG-19BBz (CAR): n=2) using image-based biodistribution. **, P<0.01.

FIGS. 5A-5B show in vivo tracking of the engineered CAR T cells of the present technology with ⁸⁶Y-DOTABn. FIG. 5A shows an exemplary scheme for tracking engineered T cells in vivo in a s.c. Raji-tumor mouse model (3×10⁶ cells) with established treatment failure. Seven days post tumor inoculation, mice were injected i.v. with either 3×10⁶ huC825-19BBz or 3×10⁶ 19BBz T cells. On day 17 post T cell administration, mice demonstrating persistent growing tumor burden indicating treatment failure were i.v. injected with ⁸⁶Y-DOTA-Bn (3.7 MBq; 40 pmol) to assess persistence and localization of the transplanted T cells. FIG. 5B shows Maximum intensity projection (MIP) and axial PET/CT images at 1, 3 and 16 h p.i. depict accumulation of huC825-19BBz-CAR T cells at the tumor (orange circle). Highest intratumoral T cell uptake was seen at 3 h pi of 4.9% ID/g (vs 0.8% ID/g in control). No uptake above background at the tumor is noted in control mice (19BBz CAR; green circle). Rapid, predominant renal tracer clearance was noted.

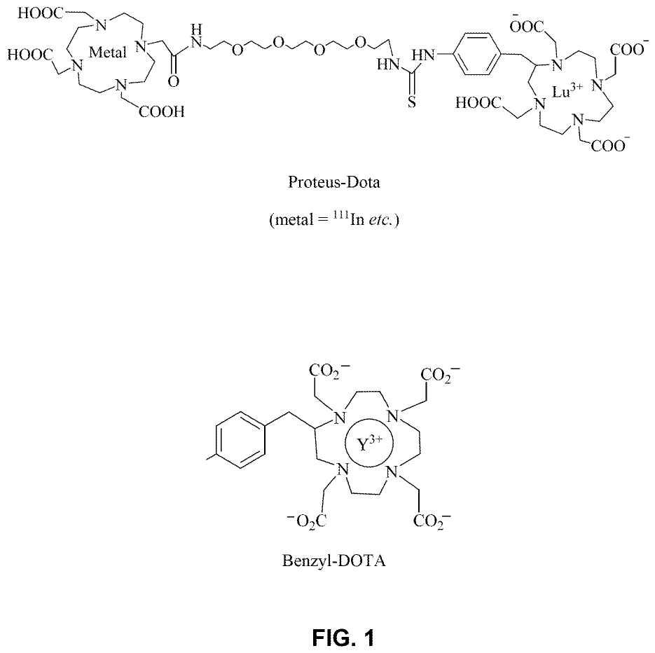

### DETAILED DESCRIPTION

It is to be appreciated that certain aspects, modes, embodiments, variations and features of the present methods are described below in various levels of detail in order to provide a substantial understanding of the present technology.

The present disclosure is not to be limited in terms of the particular embodiments described in this application, which are intended as single illustrations of individual aspects of the disclosure. All the various embodiments of the present disclosure will not be described herein. Many modifications and variations of the disclosure can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled.

It is to be understood that the present disclosure is not limited to particular uses, methods, reagents, compounds, compositions or biological systems, which can, of course, vary. It is also to be understood that the terminology used herein is for the purpose of describing particular embodiments only, and is not intended to be limiting.

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

The compositions of the present technology include engineered immune cells that express a tumor antigen-targeted chimeric antigen receptor and an anti-DOTA C825 antigen binding fragment that are useful for determining the in vivo biodistribution, viability, and expansion of the engineered immune cells described herein.

### Definitions

Unless defined otherwise, all technical and scientific terms used herein have the meaning commonly understood by a person skilled in the art to which this disclosure belongs. The following references provide one of skill with a general definition of many of the terms used in the present disclosure. Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

As used herein, the singular forms “a”, “an” and “the” are intended to include the plural forms as well, unless the context clearly indicates otherwise.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value.

As used herein, the term “administration” of an agent to a subject includes any route of introducing or delivering the agent to a subject to perform its intended function. Administration can be carried out by any suitable route, including, but not limited to, intravenously, intramuscularly, intraperitoneally, subcutaneously, and other suitable routes as described herein. Administration includes self-administration and the administration by another.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refer to agents that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. In some embodiments, amino acids forming a polypeptide are in the D form. In some embodiments, the amino acids forming a polypeptide are in the L form. In some embodiments, a first plurality of amino acids forming a polypeptide are in the D form, and a second plurality of amino acids are in the L form.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter code.

As used herein, the term “analog” refers to a structurally related polypeptide or nucleic acid molecule having the function of a reference polypeptide or nucleic acid molecule.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983)). Antibodies may comprise whole native antibodies, monoclonal antibodies, human antibodies, humanized antibodies, camelised antibodies, multispecific antibodies, bispecific antibodies, chimeric antibodies, Fab, Fab′, single chain V region fragments (scFv), single domain antibodies (e.g., nanobodies and single domain camelid antibodies), VNAR fragments, Bi-specific T-cell engager (BiTE) antibodies, minibodies, disulfide-linked Fvs (sdFv), and anti-idiotypic (anti-Id) antibodies, intrabodies, fusion polypeptides, unconventional antibodies and antigen binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA, and IgY), class (e.g., IgG1, IgG2, IgG3, IgG4, IgA1, and IgA2), or subclass.

In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, C_{H}1, C_{H}2, and C_{H}3. Each light chain is comprised of a light chain variable region (abbreviated herein as V_{L}) and a light chain constant C_{L} region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Cl q) of the classical complement system. As used herein interchangeably, the terms “antigen binding portion”, “antigen binding fragment”, or “antigen binding region” of an antibody, refer to the region or portion of an antibody that binds to the antigen and which confers antigen specificity to the antibody; fragments of antigen binding proteins, for example antibodies, include one or more fragments of an antibody that retain the ability to specifically bind to an antigen (e.g., an peptide/HLA complex). It has been shown that the antigen binding function of an antibody can be performed by fragments of a full-length antibody. Examples of antigen binding portions encompassed within the term “antibody fragments” of an antibody include a Fab fragment, a monovalent fragment consisting of the V_{L}, V_{H}, C_{L} and C_{H}1 domains; a F(ab)₂ fragment, a bivalent fragment comprising two Fab fragments linked by a disulfide bridge at the hinge region; a Fd fragment consisting of the V_{H} and C_{H}1 domains; a Fv fragment consisting of the V_{L} and V_{H} domains of a single arm of an antibody; a dAb fragment (Ward et al., Nature 341 544-546 (1989)), which consists of a V_{H} domain; and an isolated complementarity determining region (CDR). An “isolated antibody” or “isolated antigen binding protein” is one which has been identified and separated and/or recovered from a component of its natural environment. “Synthetic antibodies” or “recombinant antibodies” are generally generated using recombinant technology or using peptide synthetic techniques known to those of skill in the art.

Antibodies and antibody fragments can be wholly or partially derived from mammals (e.g., humans, non-human primates, goats, guinea pigs, hamsters, horses, mice, rats, rabbits and sheep) or non-mammalian antibody producing animals (e.g., chickens, ducks, geese, snakes, and urodele amphibians). The antibodies and antibody fragments can be produced in animals or produced outside of animals, such as from yeast or phage (e.g., as a single antibody or antibody fragment or as part of an antibody library).

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules. These are known as single chain Fv (scFv); see e.g., Bird et al., Science 242:423-426 (1988); and Huston et al., Proc. Natl. Acad. Sci. 85: 5879-5883 (1988). These antibody fragments are obtained using conventional techniques known to those of ordinary skill in the art, and the fragments are screened for utility in the same manner as are intact antibodies.

As used herein, an “antigen” refers to a molecule to which an antibody can selectively bind. The target antigen may be a protein (e.g., an antigenic peptide), carbohydrate, nucleic acid, lipid, hapten, or other naturally occurring or synthetic compound. An antigen may also be administered to an animal subject to generate an immune response in the subject.

By “binding affinity” is meant the strength of the total noncovalent interactions between a single binding site of a molecule (e.g., an antibody) and its binding partner (e.g., an antigen). Without wishing to be bound by theory, affinity depends on the closeness of stereochemical fit between antibody combining sites and antigen determinants, on the size of the area of contact between them, and on the distribution of charged and hydrophobic groups. Affinity also includes the term “avidity,” which refers to the strength of the antigen-antibody bond after formation of reversible complexes (e.g., either monovalent or multivalent). Methods for calculating the affinity of an antibody for an antigen are known in the art, comprising use of binding experiments to calculate affinity. The affinity of a molecule X for its partner Y can generally be represented by the dissociation constant (K_{d}). A low-affinity complex contains an antibody that generally tends to dissociate readily from the antigen, whereas a high-affinity complex contains an antibody that generally tends to remain bound to the antigen for a longer duration. Antibody activity in functional assays (e.g., flow cytometry assay) is also reflective of antibody affinity. Antibodies and affinities can be phenotypically characterized and compared using functional assays (e.g., flow cytometry assay).

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. Sequences of Proteins of Immunological Interest, Fifth Edition, U.S. Department of Health and Human Services, NIH Publication No. 91-3242(1991)).

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells, at least about 10,000 cells, at least about 100,000 cells, at least about 1×10⁶ cells, at least about 1×10⁷ cells, at least about 1×10⁸ cells, at least about 1×10⁹ cells, at least about 1×10¹⁰ cells, at least about 1×10¹¹ cells, at least about 1×10¹² cells, or more cells expressing similar or different phenotypes.

As used herein, the term “chimeric co-stimulatory receptor” or “CCR” refers to a chimeric receptor that binds to an antigen and provides co-stimulatory signals, but does not provide a T-cell activation signal.

As used herein, the term “conservative sequence modification” refers to an amino acid modification that does not significantly affect or alter the binding characteristics of the presently disclosed CAR (e.g., the extracellular antigen binding domain of the CAR) comprising the amino acid sequence. Conservative modifications can include amino acid substitutions, additions, and deletions. Modifications can be introduced into the human scFv of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine; negatively-charged amino acids include aspartic acid and glutamic acid; and neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

As used herein, a “control” is an alternative sample used in an experiment for comparison purpose. A control can be “positive” or “negative.”

As used herein, the term, “co-stimulatory signaling domain,” or “co-stimulatory domain”, refers to the portion of the CAR comprising the intracellular domain of a co-stimulatory molecule. Co-stimulatory molecules are cell surface molecules other than antigen receptors or Fc receptors that provide a second signal required for efficient activation and function of T lymphocytes upon binding to antigen. Examples of such co-stimulatory molecules include CD27, CD28, 4-1BB (CD137), OX40 (CD134), CD30, CD40, PD-1, ICOS (CD278), LFA-1, CD2, CD7, LIGHT, NKD2C, B7-H2 and a ligand that specifically binds CD83. Accordingly, while the present disclosure provides exemplary costimulatory domains derived from CD28 and 4-1BB, other costimulatory domains are contemplated for use with the CARs described herein. The inclusion of one or more co-stimulatory signaling domains can enhance the efficacy and expansion of T cells expressing CAR receptors. The intracellular signaling and co-stimulatory signaling domains can be linked in any order in tandem to the carboxyl terminus of the transmembrane domain.

As used herein, the term “disease” refers to any condition or disorder that damages or interferes with the normal function of a cell, tissue, or organ. Examples of diseases include neoplasia or pathogen infection of cell.

As used herein, the term “effective amount” refers to a quantity of an agent sufficient to achieve a beneficial or desired result upon administration. The amount of an agent administered to the subject can depend on the characteristics of the individual, such as general health, age, sex, body weight, effective concentration of the engineered immune cells administered, and tolerance to drugs. The skilled artisan will be able to determine appropriate dosages depending on these and other factors. An effective amount can be administered to a subject in one or more doses.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell. The expression level of a gene can be determined by measuring the amount of mRNA or protein in a cell or tissue sample. In one aspect, the expression level of a gene from one sample can be directly compared to the expression level of that gene from a control or reference sample. In another aspect, the expression level of a gene from one sample can be directly compared to the expression level of that gene from the same sample following administration of the compositions disclosed herein. The term “expression” also refers to one or more of the following events: (1) production of an RNA template from a DNA sequence (e.g., by transcription) within a cell; (2) processing of an RNA transcript (e.g., by splicing, editing, 5′ cap formation, and/or 3′ end formation) within a cell; (3) translation of an RNA sequence into a polypeptide or protein within a cell; (4) post-translational modification of a polypeptide or protein within a cell; (5) presentation of a polypeptide or protein on the cell surface; and (6) secretion or presentation or release of a polypeptide or protein from a cell. The level of expression of a polypeptide can be assessed using any method known in art, including, for example, methods of determining the amount of the polypeptide produced from the host cell. Such methods can include, but are not limited to, quantitation of the polypeptide in the cell lysate by ELISA, Coomassie blue staining following gel electrophoresis, Lowry protein assay and Bradford protein assay.

As used herein, “F(ab)” refers to a fragment of an antibody structure that binds to an antigen but is monovalent and does not have a Fc portion, for example, an antibody digested by the enzyme papain yields two F(ab) fragments and an Fc fragment (e.g., a heavy (H) chain constant region; Fc region that does not bind to an antigen).

As used herein, “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)₂” fragment can be split into two individual Fab′ fragments.

As used herein, the term “heterologous nucleic acid molecule or polypeptide” refers to a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

As used herein, a “host cell” is a cell that is used to receive, maintain, reproduce and amplify a vector. A host cell also can be used to express the polypeptide encoded by the vector. The nucleic acid contained in the vector is replicated when the host cell divides, thereby amplifying the nucleic acids.

As used herein, the term “immune cell” refers to any cell that plays a role in the immune response of a subject. Immune cells are of hematopoietic origin, and include lymphocytes, such as B cells and T cells; natural killer cells; myeloid cells, such as monocytes, macrophages, dendritic cells, eosinophils, neutrophils, mast cells, basophils, and granulocytes. As used herein, the term “engineered immune cell” refers to an immune cell that is genetically modified. As used herein, the term “native immune cell” refers to an immune cell that naturally occurs in the immune system.

As used herein, the term “increase” means to alter positively by at least about 5%, including, but not limited to, alter positively by about 5%, by about 10%, by about 25%, by about 30%, by about 50%, by about 75%, or by about 100%.

As used herein, the term “isolated cell” refers to a cell that is separated from the molecular and/or cellular components that naturally accompany the cell.

As used herein, the term “isolated,” “purified,” or “biologically pure” refers to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or polypeptide of the presently disclosed subject matter is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

As used herein, the term “ligand” refers to a molecule that binds to a receptor. In particular, the ligand binds a receptor on another cell, allowing for cell-to-cell recognition and/or interaction.

The term “linker” refers to synthetic sequences (e.g., amino acid sequences) that connect or link two sequences, e.g., that link two polypeptide domains. In some embodiments, the linker contains 1, 2, 3, 4, 5, 6, 7, 8, 9, or 10 amino acid residues.

The term “lymphocyte” refers to all immature, mature, undifferentiated, and differentiated white blood cell populations that are derived from lymphoid progenitors including tissue specific and specialized varieties, and encompasses, by way of non-limiting example, B cells, T cells, NKT cells, and NK cells. In some embodiments, lymphocytes include all B cell lineages including pre-B cells, progenitor B cells, early pro-B cells, late pro-B cells, large pre-B cells, small pre-B cells, immature B cells, mature B cells, plasma B cells, memory B cells, B-1 cells, B-2 cells, and anergic AN1/T3 cell populations.

As used herein, the term “modulate” means to positively or negatively alter. Exemplary modulations include an about 1%, about 2%, about 5%, about 10%, about 25%, about 50%, about 75%, or about 100% change.

As used herein, the term “neoplasia” refers to a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasias can affect a variety of cell types, tissues, or organs, including but not limited to an organ selected from the group consisting of bladder, colon, bone, brain, breast, cartilage, glia, esophagus, fallopian tube, gallbladder, heart, intestines, kidney, liver, lung, lymph node, nervous tissue, ovaries, pleura, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasias include cancers, such as sarcomas, carcinomas, or plasmacytomas (malignant tumor of the plasma cells).

As used herein, “operably linked” with reference to nucleic acid sequences, regions, elements or domains means that the nucleic acid regions are functionally related to each other. For example, nucleic acid encoding a leader peptide can be operably linked to nucleic acid encoding a polypeptide, whereby the nucleic acids can be transcribed and translated to express a functional fusion protein, wherein the leader peptide effects secretion of the fusion polypeptide. In some instances, the nucleic acid encoding a first polypeptide (e.g., a leader peptide) is operably linked to nucleic acid encoding a second polypeptide and the nucleic acids are transcribed as a single mRNA transcript, but translation of the mRNA transcript can result in one of two polypeptides being expressed. For example, an amber stop codon can be located between the nucleic acid encoding the first polypeptide and the nucleic acid encoding the second polypeptide, such that, when introduced into a partial amber suppressor cell, the resulting single mRNA transcript can be translated to produce either a fusion protein containing the first and second polypeptides, or can be translated to produce only the first polypeptide. In another example, a promoter can be operably linked to nucleic acid encoding a polypeptide, whereby the promoter regulates or mediates the transcription of the nucleic acid.

As used herein, the “percent homology” between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4: 1 1-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

The terms “polypeptide,” “peptide,” and “protein” are used interchangeably herein to refer to a polymer of amino acid residues. The terms apply to naturally occurring amino acid polymers as well as amino acid polymers in which one or more amino acid residues are a non-naturally occurring amino acid, e.g., an amino acid analog. The terms encompass amino acid chains of any length, including full length proteins, wherein the amino acid residues are linked by covalent peptide bonds.

As used herein, the term “reduce” means to alter negatively by at least about 5% including, but not limited to, alter negatively by about 5%, by about 10%, by about 25%, by about 30%, by about 50%, by about 75%, or by about 100%.

As used herein, “regulatory region” of a nucleic acid molecule means a cis-acting nucleotide sequence that influences expression, positively or negatively, of an operably linked gene. Regulatory regions include sequences of nucleotides that confer inducible (i.e., require a substance or stimulus for increased transcription) expression of a gene. When an inducer is present or at increased concentration, gene expression can be increased. Regulatory regions also include sequences that confer repression of gene expression (i.e., a substance or stimulus decreases transcription). When a repressor is present or at increased concentration gene expression can be decreased. Regulatory regions are known to influence, modulate or control many in vivo biological activities including cell proliferation, cell growth and death, cell differentiation and immune modulation. Regulatory regions typically bind to one or more trans-acting proteins, which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to and including 10 Kb. Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more. Regulatory regions also include, but are not limited to, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding site (IRES) elements for the creation of multigene, or polycistronic, messages, polyadenylation signals to provide proper polyadenylation of the transcript of a gene of interest and stop codons, and can be optionally included in an expression vector.

As used herein, the term “sample” refers to clinical samples obtained from a subject. In certain embodiments, a sample is obtained from a biological source (i.e., a “biological sample”), such as tissue, bodily fluid, or microorganisms collected from a subject. Sample sources include, but are not limited to, mucus, sputum, bronchial alveolar lavage (BAL), bronchial wash (BW), whole blood, bodily fluids, cerebrospinal fluid (CSF), urine, plasma, serum, or tissue.

As used herein, the term “secreted” in reference to a polypeptide means a polypeptide that is released from a cell via the secretory pathway through the endoplasmic reticulum, Golgi apparatus, and as a vesicle that transiently fuses at the cell plasma membrane, releasing the proteins outside of the cell. Small molecules, such as drugs, can also be secreted by diffusion through the membrane to the outside of cell.

As used herein, the term “single-chain variable fragment” or “scFv” is a fusion protein of the variable regions of the heavy (V_{H}) and light chains (V_{L}) of an immunoglobulin (e.g., mouse or human) covalently linked to form a V_{H}::V_{L} heterodimer. The heavy (V_{H}) and light chains (V_{L}) are either joined directly or joined by a peptide-encoding linker (e.g., about 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. The linker can link the heavy chain variable region and the light chain variable region of the extracellular antigen binding domain. In certain embodiments, the linker comprises amino acids having the sequence set forth in SEQ ID NO: 1 as provided below: GGGGSGGGGSGGGGS (SEQ ID NO: 1). In certain embodiments, the nucleic acid sequence encoding the amino acid sequence of SEQ ID NO: 1 is set forth in SEQ ID NO: 2, which is provided below: ggcggcggcggatctggaggtggtggctcaggtggcggaggctcc (SEQ ID NO: 2). Other examples of linkers include GGGGSGGGGSGGGGSGGGGS (i.e., [G4S]4) (SEQ ID NO: 33), or GGGGSGGGGSGGGGSGGGGSGGGGSGGGGS (i.e., [G4S]6) (SEQ ID NO: 34).

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston, et al. (Proc. Nat. Acad. Sci. USA, 85:5879-5883 (1988)). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hybridoma (Larchmt) 27(6):455-51 (2008); Peter et al., J Cachexia Sarcopenia Muscle (2012); Shieh et al., J Imunol 183(4):2277-85 (2009); Giomarelli et al., Thromb Haemost 97(6):955-63 (2007); Fife eta., J Clin Invst 116(8):2252-61 (2006); Brocks et al., Immunotechnology 3(3): 173-84 (1997); Moosmayer et al., Ther Immunol 2(10):31-40 (1995). Agonistic scFvs having stimulatory activity have been described (see, e.g., Peter et al., J Biol Chem 25278(38):36740-7 (2003); Xie et al., Nat Biotech 15(8):768-71 (1997); Ledbetter et al., Crit Rev Immunol 17(5-6):427-55 (1997); Ho et al., Bio Chim Biophys Acta 1638(3):257-66 (2003)).

As used herein, the term “specifically binds” or “specifically binds to” or “specifically target” refers to a molecule (e.g., a polypeptide or fragment thereof) that recognizes and binds a molecule of interest (e.g., an antigen), but which does not substantially recognize and bind other molecules. The terms “specific binding,” “specifically binds to,” or is “specific for” a particular molecule (e.g., an antigen), as used herein, can be exhibited, for example, by a molecule having a K_{d} for the molecule to which it binds to of about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M.

As used herein, the terms “subject,” “individual,” or “patient” are used interchangeably and refer to an individual organism, a vertebrate, or a mammal and may include humans, non-human primates, rodents, and the like (e.g., which is to be the recipient of a particular medical intervention, or from whom cells are harvested). In certain embodiments, the individual, patient or subject is a human.

The terms “substantially homologous” or “substantially identical” mean a polypeptide or nucleic acid molecule that exhibits at least 50% or greater homology or identity to a reference amino acid sequence (for example, any one of the amino acid sequences described herein) or nucleic acid sequence (for example, any one of the nucleic acid sequences described herein). For example, such a sequence is at least about 60%, about 65%, about 70%, about 75%, about 80%, about 85%, about 90%, about 95% or about 99% homologous or identical at the amino acid level or nucleic acid to the sequence used for comparison (e.g., a wild-type, or native, sequence). In some embodiments, a substantially homologous or substantially identical polypeptide contains one or more amino acid amino acid substitutions, insertions, or deletions relative to the sequence used for comparison. In some embodiments, a substantially homologous or substantially identical polypeptide contains one or more non-natural amino acids or amino acid analogs, including, D-amino acids and retroinverso amino, to replace homologous sequences.

Sequence homology or sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. In an exemplary approach to determining the degree of identity, a BLAST program may be used, with a probability score between e-3 and e-100 indicating a closely related sequence.

Nucleic acid molecules useful in the presently disclosed subject matter include any nucleic acid molecule that encodes a polypeptide or a fragment thereof. In certain embodiments, nucleic acid molecules useful in the presently disclosed subject matter include nucleic acid molecules that encode an antibody or an antigen binding portion thereof. Such nucleic acid molecules need not be 100% identical with an endogenous nucleic acid sequence, but will typically exhibit substantial identity. Polynucleotides having “substantial homology” or “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger, Methods Enzymol. 152:399 (1987); Kimmel, A. R. Methods Enzymol. 152:507 (1987)). For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% w/v formamide, or at least about 50% w/v formamide. Stringent temperature conditions will ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In certain embodiments, hybridization will occur at 30° C. in 750 mM NaCl, 75 mM trisodium citrate, and 1% w/v SDS. In certain embodiments, hybridization will occur at 37° C. in 500 mM NaCl, 50 mM trisodium citrate, 1% w/v SDS, 35% w/v formamide, and 100 μg/ml denatured salmon sperm DNA (ssDNA). In certain embodiments, hybridization will occur at 42° C. in 250 mM NaCl, 25 mM trisodium citrate, 1% w/v SDS, 50% w/v formamide, and 200 μg ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will less than about 30 mM NaCl and 3 mM trisodium citrate, or less than about 15 mM NaCl and 1.5 mM trisodium citrate. Stringent temperature conditions for the wash steps will ordinarily include a temperature of at least about 25° C., at least about 42° C., or at least about 68° C. In certain embodiments, wash steps will occur at 25° C. in 30 mM NaCl, 3 mM trisodium citrate, and 0.1% w/v SDS. In certain embodiments, wash steps will occur at 42° C. in 15 mM NaCl, 1.5 mM trisodium citrate, and 0.1% w/v SDS. In certain embodiments, wash steps will occur at 68° C. in 15 mM NaCl, 1.5 mM trisodium citrate, and 0.1% w/v SDS. Additional variations on these conditions will be readily apparent to those skilled in the art. Hybridization techniques are well known to those skilled in the art and are described, for example, in Benton and Davis (Science 196: 180 (1977)); Grunstein and Rogness (Proc. Natl. Acad. Sci., USA 72:3961 (1975)); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

As used herein, “synthetic,” with reference to, for example, a synthetic nucleic acid molecule or a synthetic gene or a synthetic peptide refers to a nucleic acid molecule or polypeptide molecule that is produced by recombinant methods and/or by chemical synthesis methods. As used herein, “production by recombinant means by using recombinant DNA methods” means the use of the well-known methods of molecular biology for expressing proteins encoded by cloned DNA.

As used herein, the term “T-cell” includes naïve T cells, CD4⁺ T cells, CD8⁺ T cells, memory T cells, activated T cells, anergic T cells, tolerant T cells, chimeric B cells, and antigen-specific T cells.

As used herein “tumor-infiltrating lymphocytes” or “TILs” refer to white blood cells that have left the bloodstream and migrated into a tumor.

As used herein, a “vector” is a replicable nucleic acid from which one or more heterologous proteins can be expressed when the vector is transformed into an appropriate host cell. Reference to a vector includes those vectors into which a nucleic acid encoding a polypeptide or fragment thereof can be introduced, typically by restriction digest and ligation. Reference to a vector also includes those vectors that contain nucleic acid encoding a polypeptide. The vector is used to introduce the nucleic acid encoding the polypeptide into the host cell for amplification of the nucleic acid or for expression/display of the polypeptide encoded by the nucleic acid. The vectors typically remain episomal, but can be designed to effect integration of a gene or portion thereof into a chromosome of the genome. Also contemplated are vectors that are artificial chromosomes, such as yeast artificial chromosomes and mammalian artificial chromosomes. Selection and use of such vehicles are well known to those of skill in the art. A vector also includes “virus vectors” or “viral vectors.” Viral vectors are engineered viruses that are operably linked to exogenous genes to transfer (as vehicles or shuttles) the exogenous genes into cells. As used herein, an “expression vector” includes vectors capable of expressing DNA that is operably linked with regulatory sequences, such as promoter regions, that are capable of effecting expression of such DNA fragments. Such additional segments can include promoter and terminator sequences, and optionally can include one or more origins of replication, one or more selectable markers, an enhancer, a polyadenylation signal, and the like. Expression vectors are generally derived from plasmid or viral DNA, or can contain elements of both. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

### Overview

As described herein, immune cells can be engineered to constitutively or conditionally express an anti-DOTA C825 antigen binding fragment that binds to a DOTA hapten. In some embodiments, the engineered immune cells additionally express a chimeric antigen receptor for delivering the immune cell to the target site. Expression of the anti-DOTA C825 antigen binding fragment allows for tracking of the CAR T cells and identification of tumor cell sites. Without wishing to be bound by theory, it is believed that the engineered immune cells of the present technology are useful for monitoring the in vivo distribution of the engineered immune cells over time.

The methods provided herein allow for modular use of a wide range of CAR and tumor specific antibody combinations depending on the desired application. The engineered immune cells described herein can be employed in combination with a wide variety of tumor specific antibodies. Tumor specific antibodies are known in art. Exemplary tumor specific antibodies include, but are not limited to, antibodies targeted to Her2, EGFR, PSMA, CD20, CD33, CD38, or WT1. In some embodiments, the tumor specific antibody is trastuzumab, cetuximab, ESK1, rituximab, daratumumab, or lintuzumab.

In some embodiments, the engineered immune cells provided herein express a T-cell receptor (TCR) or other cell-surface ligand that binds to a target antigen, such as a tumor antigen and an anti-DOTA C825 antigen binding fragment. In some embodiments, the T cell receptor is a wild-type, or native, T-cell receptor. In some embodiments, the T cell receptor is a chimeric T-cell receptor (CAR).

In exemplary embodiments provided herein, the engineered immune cells provided herein express a T-cell receptor (TCR) (e.g., a CAR) or other cell-surface ligand that binds to a CD19 tumor antigen. In some embodiments, the engineered immune cells provided herein express a T-cell receptor (TCR) (e.g., a CAR) or other cell-surface ligand that binds to a CD19 tumor antigen presented in the context of an MHC molecule. In some embodiments, the engineered immune cells provided herein express a T-cell receptor (TCR) (e.g., a CAR) or other cell-surface ligand that binds to a CD19 tumor antigen presented in the context of an HLA-A2 molecule.

In exemplary embodiments provided herein, the engineered immune cells provided herein express a T-cell receptor (TCR) (e.g., a CAR) or other cell-surface ligand that binds to a “preferentially expressed antigen in melanoma” (PRAME) tumor antigen. In some embodiments, the engineered immune cells provided herein express a T-cell receptor (TCR) (e.g., a CAR) or other cell-surface ligand that binds to a PRAME tumor antigen presented in the context of an MHC molecule. In some embodiments, the PRAME tumor antigen presented in the context of an HLA-A2 molecule. The PRAME protein is a currently undruggable, retinoic acid receptor binding protein involved in differentiation, proliferation arrest, and apoptosis. After proteasomal processing the PRAME³⁰⁰⁻³⁰⁹ peptide (ALYVDSLFFL) (SEQ ID NO: 32) is presented on the cell surface in the context of an HLA-I haplotype HLA*A02:01 (HLA-A2).

In exemplary embodiments provided herein, the engineered immune cells provided herein express a T-cell receptor (TCR) (e.g., a CAR) or other cell-surface ligand that binds to a Wilm's tumor protein 1 (WT1) tumor antigen. In some embodiments, the engineered immune cells provided herein express a T-cell receptor (TCR) (e.g., a CAR) or other cell-surface ligand that binds to a WT1 tumor antigen presented in the context of an MHC molecule. In some embodiments, the engineered immune cells provided herein express a T-cell receptor (TCR) (e.g., a CAR) or other cell-surface ligand that binds to a WT1 tumor antigen presented in the context of an HLA-A2 molecule.

In exemplary embodiments provided herein, the engineered immune cells provided herein express a T-cell receptor (TCR) (e.g., a CAR) or other cell-surface ligand that binds to a mesothelin tumor antigen. In some embodiments, the engineered immune cells provided herein express a T-cell receptor (TCR) (e.g., a CAR) or other cell-surface ligand that binds to a mesothelin tumor antigen presented in the context of an MHC molecule. In some embodiments, the engineered immune cells provided herein express a T-cell receptor (TCR) (e.g., a CAR) or other cell-surface ligand that binds to a mesothelin tumor antigen presented in the context of an HLA-A2 molecule.

In exemplary embodiments provided herein, the engineered immune cells provided herein express a T-cell receptor (TCR) (e.g., a CAR) or other cell-surface ligand that binds to a MUC16 tumor antigen. In some embodiments, the engineered immune cells provided herein express a T-cell receptor (TCR) (e.g., a CAR) or other cell-surface ligand that binds to a MUC16 tumor antigen presented in the context of an MHC molecule. In some embodiments, the engineered immune cells provided herein express a T-cell receptor (TCR) (e.g., a CAR) or other cell-surface ligand that binds to a MUC16 tumor antigen presented in the context of an HLA-A2 molecule.

In exemplary embodiments provided herein, the engineered immune cells provided herein express a T-cell receptor (TCR) (e.g., a CAR) or other cell-surface ligand that binds to a prostate stem cell antigen (PSCA) tumor antigen. In some embodiments, the engineered immune cells provided herein express a T-cell receptor (TCR) (e.g., a CAR) or other cell-surface ligand that binds to a PSCA tumor antigen presented in the context of an MHC molecule. In some embodiments, the engineered immune cells provided herein express a T-cell receptor (TCR) (e.g., a CAR) or other cell-surface ligand that binds to a PSCA tumor antigen presented in the context of an HLA-A2 molecule.

In exemplary embodiments provided herein, the engineered immune cells provided herein express a T-cell receptor (TCR) (e.g., a CAR) or other cell-surface ligand that binds to a B cell maturation antigen (BCMA) tumor antigen. In some embodiments, the engineered immune cells provided herein express a T-cell receptor (TCR) (e.g., a CAR) or other cell-surface ligand that binds to a BCMA tumor antigen presented in the context of an MHC molecule. In some embodiments, the engineered immune cells provided herein express a T-cell receptor (TCR) (e.g., a CAR) or other cell-surface ligand that binds to a BCMA tumor antigen presented in the context of an HLA-A2 molecule.

The engineered immune cells (e.g., CAR T cells) provided herein that express an antigen receptor, e.g., a chimeric antigen receptor, in combination with an anti-DOTA C825 antigen binding fragment are useful for monitoring the in vivo distribution of the engineered immune cells over time.

In addition, the engineered immune cells will proliferate extensively (e.g., 100 times or more) when it encounters the tumor specific antigen at the tumor site, thus significantly increasing production of the anti-DOTA C825 antigen binding fragment. The engineered immune cells (e.g., CAR T cells) can be easily generated by in vitro transduction of immune cells with nucleic acid encoding the chimeric antigen and the anti-DOTA C825 antigen binding fragment.

The amino acid sequence of the V_{H} of the anti-DOTA C825 antigen binding fragment may be:

(SEQ ID NO: 35)
HVQLVESGGGLVQPGGSLRLSCAASGFSLTDYGVHWVRQAPGKGLEWLGV
IWSGGGTAYNTALISRFTISRDNSKNTLYLQMNSLRAEDTAVYYCARRGS
YPYNYFDAWGCGTLVTVSS

The amino acid sequence of the V_{L} of the anti-DOTA C825 antigen binding fragment may be:

(SEQ ID NO: 36)
QAVVTQEPSLTVSPGGTVTLTCGSSTGAVTASNYANWVQQKPGQCPRGLI
GGHNNRPPGVPARFSGSLLGGKAALTLLGAQPEDEAEYYCALWYSDHWVI
GGGTKLTVLG

The anti-DOTA C825 antigen binding fragment may comprise an amino acid sequence selected from the group consisting of:

(SEQ ID NO: 37)
HVQLVESGGGLVQPGGSLRLSCAASGFSLTDYGVHWVRQA
PGKGLEWLGVIWSGGGTAYNTALISRFTISRDNSKNTLYL
QMNSLRAEDTAVYYCARRGSYPYNYFDAWGCGTLVTVSSG
GGGSGGGGSGGGGSQAVVTQEPSLTVSPGGTVTLTCGSST
GAVTASNYANWVQQKPGQCPRGLIGGHNNRPPGVPARFSG
SLLGGKAALTLLGAQPEDEAEYYCALWYSDHWVIGGGTKL
TVLG;
and
(SEQ ID NO: 38)
HVQLVESGGGLVQPGGSLRLSCAASGFSLTDYGVHWVRQA
PGKGLEWLGVIWSGGGTAYNTALISRFTISRDNSKNTLYL
QMNSLRAEDTAVYYCARRGSYPYNYFDAWGCGTLVTVSSG
GGGSGGGGSGGGGSGGGGSGGGGSGGGGSQAVVTQEPSLT
VSPGGTVTLTCGSSTGAVTASNYANWVQQKPGQCPRGLIG
GHNNRPPGVPARFSGSLLGGKAALTLLGAQPEDEAEYYCA
LWYSDHWVIGGGTKLTVLG

*(G4S)3 linker sequence (SEQ ID NO: 1) is shown in boldface

In some embodiments, the anti-DOTA C825 antigen binding fragment is an scFv, a Fab, or a (Fab)₂.

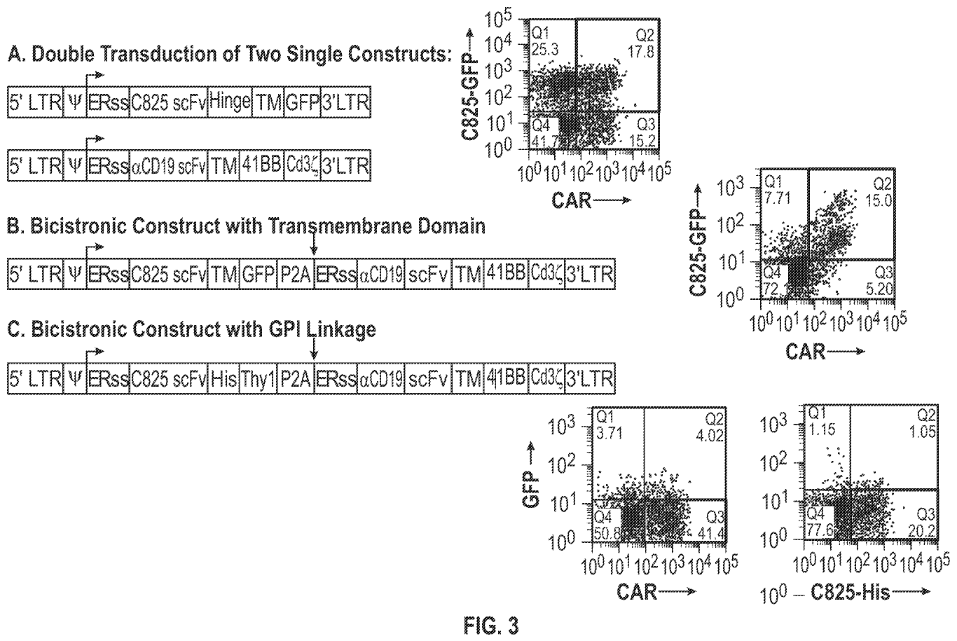

Exemplary constructs of the present technology include double transduction constructs such as those described in FIG. 3A. The amino acid sequences of the constructs described in FIG. 3A are shown below:

C825-hinge-GFP
(SEQ ID NO: 39)
TMNRGVPFRHLLLVLQLALLPAATQGHVQLVESGGGLVQP
GGSLRLSCAASGFSLTDYGVHWVRQAPGKGLEWLGVIWSG
GGTAYNTALISRFTISRDNSKNTLYLQMNSLRAEDTAVYY
CARRGSYPYNYFDAWGCGTLVTVSSGGGGSGGGGSGGGGS
QAVVTQEPSLTVSPGGTVTLTCGSSTGAVTASNYANWVQQ
KPGQCPRGLIGGHNNRPPGVPARFSGSLLGGKAALTLLGA
QPEDEAEYYCALWYSDHWVIGGGTKLTVLGDLEPKSPDKT
HTCPPCPAPPVAGPSVFLFPPKPKDTLMISRTPEVTCVVV
DVSHEDPEVKFNWYVDGVEVHNAKTKPREEQYQSTYRVVS
VLTVLHQDWLNGKEYKCKVSNKALPAPIEKTISKAKGQPR
EPQVYTLPPSRDELTKNQVSLTCLVKGFYPSDIAVEWESN
GQPENNYKTTPPVLDSDGSFFLYSKLTVDKSRWQQGNVFS
CSVMHEALHNHYTQKSLSLSPGKKDPKAAAMALIVLGGVA
GLLLFIGLGIFFCVRCRHMVSKGEELFTGVVPILVELDGD
VNGHKFSVSGEGEGDATYGKLTLKFICTTGKLPVPWPTLV
TTLTYGVQCFSRYPDHMKQHDFFKSAMPEGYVQERTIFFK
DDGNYKTRAEVKFEGDTLVNRIELKGIDFKEDGNILGHKL
EYNYNSHNVYIMADKQKNGIKVNFKIRHNIEDGSVQLADH
YQQNTPIGDGPVLLPDNHYLSTQSALSKDPNEKRDHMVLL
EFVTAAGITLGMDELYK

* The V_{H} and V_{L} sequences of the C825 scFv are underlined, (G4S)3 linker sequence (SEQ ID NO: 1) is italicized, and transmembrane domain is in boldface.

19BBz CAR
(SEQ ID NO: 40)
MALPVTALLLPLALLLHAEVKLQQSGAELVRPGSSVKISC
KASGYAFSSYWMNWVKQRPGQGLEWIGQIYPGDGDTNYNG
KFKGQATLTADKSSSTAYMQLSGLTSEDSAVYFCARKTIS
SVVDFYFDYWGQGTTVTVSSGGGGSGGGGSGGGGSDIELT
QSPKFMSTSVGDRVSVTCKASQNVGTNVAWYQQKPGQSPK
PLIYSATYRNSGVPDRFTGSGSGTDFTLTITNVQSKDLAD
YFCQQYNRYPYTSGGGTKLEIKRAAAPTTTPAPRPPTPAP
TIASQPLSLRPEACRPAAGGAVHTRGLDFACDIYIWAPLA
GTCGVLLLSLVITLYCNKRGRKKLLYIFKQPFMRPVQTTQ
EEDGCSCRFPEEEEGGCELRVKFSRSADAPAYQQGQNQLY
NELNLGRREEYDVLDKRRGRDPEMGGKPRRKNPQEGLYNE
LQKDKMAEAYSEIGMKGERRRGKGHDGLYQGLSTATKDTY
DALHMQALPPR

* The V_{H} and V_{L} sequences of the CD19 scFv are underlined, (G4S)3 linker sequence (SEQ ID NO: 1) is italicized, and the transmembrane domain is in boldface, 41BB is italicized and underlined, and CD3ζ polypeptide is underlined and in boldface.

Other exemplary constructs of the present technology include those described in FIGS. 3B-3C. The amino acid sequences of the constructs described in FIGS. 3B-3C are shown below:

(SEQ ID NO: 41)
TMNRGVPFRHLLLVLQLALLPAATQGHVQLVESGGGLVQP
GGSLRLSCAASGFSLTDYGVHWVRQAPGKGLEWLGVIWSG
GGTAYNTALISRFTISRDNSKNTLYLQMNSLRAEDTAVYY
CARRGSYPYNYFDAWGCGTLVTVSSGGGGSGGGGSGGGGS
QAVVTQEPSLTVSPGGTVTLTCGSSTGAVTASNYANWVQQ
KPGQCPRGLIGGHNNRPPGVPARFSGSLLGGKAALTLLGA
QPEDEAEYYCALWYSDHWVIGGGTKLTVLGKDPKAAAMAL
IVLGGVAGLLLFIGLGIFFCVRCRHMVSKGEELFTGVVPI
LVELDGDVNGHKFSVSGEGEGDATYGKLTLKFICTTGKLP
VPWPTLVTTLTYGVQCFSRYPDHMKQHDFFKSAMPEGYVQ
ERTIFFKDDGNYKTRAEVKFEGDTLVNRIELKGIDFKEDG
NILGHKLEYNYNSHNVYIMADKQKNGIKVNFKIRHNIEDG
SVQLADHYQQNTPIGDGPVLLPDNHYLSTQSALSKDPNEK
RDHMVLLEFVTAAGITLGMDELYKATNFSLLKQAGDVEEN
PGPALPVTALLLPLALLLHAEVKLQQSGAELVRPGSSVKI
SCKASGYAFSSYWMNWVKQRPGQGLEWIGQIYPGDGDTNY
NGKFKGQATLTADKSSSTAYMQLSGLTSEDSAVYFCARKT
ISSVVDFYFDYWGQGTTVTVSSGGGGSGGGGSGGGGSDIE
LTQSPKFMSTSVGDRVSVTCKASQNVGTNVAWYQQKPGQS
PKPLIYSATYRNSGVPDRFTGSGSGTDFTLTITNVQSKDL
ADYFCQQYNRYPYTSGGGTKLEIKRAAAPTTTPAPRPPTP
APTIASQPLSLRPEACRPAAGGAVHTRGLDFACDIYIWAP
LAGTCGVLLLSLVITLYCNKRGRKKLLYIFKQPFMRPVQT
TQEEDGCSCRFPEEEEGGCELRVKFSRSAEPPAYQQGQNQ
LYNELNLGRREEYDVLDKRRGRDPEMGGKPRRKNPQEGLY
NELQKDKMAEAYSEIGMKGERRRGKGHDGLYQGLSTATKD
TYDALHMQALPPR
(SEQ ID NO: 42)
TMNRGVPFRHLLLVLQLALLPAATQGHVQLVESGGGLVQP
GGSLRLSCAASGFSLTDYGVHWVRQAPGKGLEWLGVIWSG
GGTAYNTALISRFTISRDNSKNTLYLQMNSLRAEDTAVYY
CARRGSYPYNYFDAWGCGTLVTVSSGGGGSGGGGSGGGGS
QAVVTQEPSLTVSPGGTVTLTCGSSTGAVTASNYANWVQQ
KPGQCPRGLIGGHNNRPPGVPARFSGSLLGGKAALTLLGA
QPEDEAEYYCALWYSDHWVIGGGTKLTVLGHHHHHHDKLV
KCEGISLLAQNTSWLLLLLLSLSLLQATDFMSLATNFSLL
KQAGDVEENPGPMALPVTALLLPLALLLHAEVKLQQSGAE
LVRPGSSVKISCKASGYAFSSYWMNWVKQRPGQGLEWIGQ
IYPGDGDTNYNGKFKGQATLTADKSSSTAYMQLSGLTSED
SAVYFCARKTISSVVDFYFDYWGQGTTVTVSSGGGGSGGG
GSGGGGSDIELTQSPKFMSTSVGDRVSVTCKASQNVGTNV
AWYQQKPGQSPKPLIYSATYRNSGVPDRFTGSGSGTDFTL
TITNVQSKDLADYFCQQYNRYPYTSGGGTKLEIKRAAAPT
TTPAPRPPTPAPTIASQPLSLRPEACRPAAGGAVHTRGLD
FACDIYIWAPLAGTCGVLLLSLVITLYCNKRGRKKLLYIF
KQPFMRPVQTTQEEDGCSCRFPEEEEGGCELRVKFSRSAD
APAYQQGQNQLYNELNLGRREEYDVLDKRRGRDPEMGGKP
RRKNPQEGLYNELQKDKMAEAYSEIGMKGERRRGKGHDGL
YQGLSTATKDTYDALHMQALPPR

* The V_{H} and V_{L} sequences of the C825 scFv and CD19 scFv are underlined, (G4S)3 linker sequence (SEQ ID NO: 1) is italicized, the transmembrane domains are in boldface, 41BB is italicized and underlined, and CD3ζ polypeptide is underlined and in boldface.

### Targeting Ligands and Target Antigens

In some embodiments, the engineered immune cells provided herein express a T-cell receptor (TCR) or other cell-surface ligand that binds to a target antigen, such as a tumor antigen. The cell-surface ligand can be any molecule that directs an immune cell to a target site (e.g., a tumor site). Exemplary cell surface ligands include, for example endogenous receptors, engineered receptors, or other specific ligands to achieve targeting of the immune cell to a target site. In some embodiments, the receptor is a T cell receptor. In some embodiments, the T cell receptor is a wild-type, or native, T-cell receptor that binds to a target antigen. In some embodiments, the receptor, e.g., a T cell receptor, is non-native receptor (e.g., not endogenous to the immune cells). In some embodiments, the receptor is a chimeric antigen receptor (CAR), for example, a T cell CAR, that binds to a target antigen.

In some embodiments, the target antigen expressed by a tumor cell. In some embodiments, the target antigen is expressed on the surface of a tumor cell. In some embodiments, the target antigen is a cell surface receptor. In some embodiments, the target antigen is a cell surface glycoprotein. In some embodiments, the target antigen is secreted by a tumor cell. In some embodiments, the target antigen is localized to the tumor microenvironment. In some embodiments, the target antigen is localized to the extracellular matrix or stroma of the tumor microenvironment. In some embodiments, the target antigen is expressed by one or more cells located within the extracellular matrix or stroma of the tumor microenvironment.

In some embodiments, the target antigen is a tumor antigen selected from among 5T4, alpha 5β1-integrin, 707-AP, A33, AFP, ART-4, B7H4, BAGE, Bcl-2, β-catenin, BCMA, Bcr-abl, MN/C IX antibody, CA125, CA19-9, CAMEL, CAP-1, CASP-8, CD4, CD5, CD19, CD20, CD21, CD22, CD25, CDC27/m, CD33, CD37, CD45, CD52, CD56, CD80, CD123, CDK4/m, CEA, c-Met, CS-1, CT, Cyp-B, cyclin B1, DAGE, DAM, EBNA, EGFR, ErbB3, ELF2M, EMMPRIN, EpCam, ephrinB2, estrogen receptor, ETV6-AML1, FAP, ferritin, folate-binding protein, GAGE, G250, GD-2, GM2, GnT-V, gp75, gp100 (Pmel 17), HAGE, HER-2/neu, HLA-A*0201-R170I, HPV E6, HPV E7, Ki-67, HSP70-2M, HST-2, hTERT (or hTRT), iCE, IGF-1R, IL-2R, IL-5, KIAA0205, LAGE, LDLR/FUT, LRP, MAGE, MART, MART-1/melan-A, MART-2/Ski, MC1R, mesothelin, MUC, MUC16, MUM-1-B, myc, MUM-2, MUM-3, NA88-A, NYESO-1, NY-Eso-B, p53, proteinase-3, p190 minor bcr-abl, Pml/RARα, PRAME, progesterone receptor, PSA, PSCA, PSM, PSMA, ras, RAGE, RU1 or RU2, RORI, SART-1 or SART-3, survivin, TEL/AML1, TGFβ, TPI/m, TRP-1, TRP-2, TRP-2/INT2, tenascin, TSTA tyrosinase, VEGF, and WT1. In certain embodiments, the target antigen is a tumor antigen selected from among BCMA, CD19, mesothelin, MUC16, PSCA, WT1, and PRAME.

In some embodiments, target antigen is a tumor antigen presented in the context of an MHC molecule. In some embodiments, the MHC protein is a MHC class I protein. In some embodiments, the MHC Class I protein is an HLA-A, HLA-B, or HLA-C molecules. In some embodiments, target antigen is a tumor antigen presented in the context of an HLA-A2 molecule. IgG1, afucosylated Fc forms, bispecific, BiTE, and CAR T cell formats or portions thereof can be employed as described herein for the recognition of target antigens present on the surface of a target cell (e.g., a tumor cell) in the context of an MHC molecule.

### Chimeric Antigen Receptors

In some embodiments, the engineered immune cells provided herein express at least one chimeric antigen receptor (CAR). CARs are engineered receptors, which graft or confer a specificity of interest onto an immune effector cell. For example, CARs can be used to graft the specificity of a monoclonal antibody onto an immune cell, such as a T cell. In some embodiments, transfer of the coding sequence of the CAR is facilitated by nucleic acid vector, such as a retroviral vector.

There are currently three generations of CARs. In some embodiments, the engineered immune cells provided herein express a “first generation” CAR. “First generation” CARs are typically composed of an extracellular antigen binding domain (e.g., a single-chain variable fragment (scFv)) fused to a transmembrane domain fused to cytoplasmic/intracellular domain of the T cell receptor (TCR) chain. “First generation” CARs typically have the intracellular domain from the CD3ζ chain, which is the primary transmitter of signals from endogenous TCRs. “First generation” CARs can provide de novo antigen recognition and cause activation of both CD4⁺ and CD8⁺ T cells through their CD3ζ chain signaling domain in a single fusion molecule, independent of HLA-mediated antigen presentation.

In some embodiments, the engineered immune cells provided herein express a “second generation” CAR. “Second generation” CARs add intracellular domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (e.g., CD3ζ).

In some embodiments, the engineered immune cells provided herein express a “third generation” CAR. “Third generation” CARs comprise those that provide multiple co-stimulation (e.g., CD28 and 4-1BB) and activation (e.g., CD3ζ).

In accordance with the presently disclosed subject matter, the CARs of the engineered immune cells provided herein comprise an extracellular antigen-binding domain, a transmembrane domain and an intracellular domain.

Extracellular Antigen-Binding Domain of a CAR. In certain embodiments, the extracellular antigen-binding domain of a CAR specifically binds a tumor antigen. In certain embodiments, the extracellular antigen-binding domain is derived from a monoclonal antibody (mAb) that binds to a tumor antigen. In some embodiments, the extracellular antigen-binding domain comprises an scFv. In some embodiments, the extracellular antigen-binding domain comprises a Fab, which is optionally crosslinked. In a some embodiments, the extracellular binding domain comprises a F(ab)₂. In some embodiments, any of the foregoing molecules are comprised in a fusion protein with a heterologous sequence to form the extracellular antigen-binding domain. In certain embodiments, the extracellular antigen-binding domain comprises a human scFv that binds specifically to a tumor antigen. In certain embodiments, the scFv is identified by screening scFv phage library with tumor antigen-Fc fusion protein.

In certain embodiments, the extracellular antigen-binding domain of a presently disclosed CAR has a high binding specificity and high binding affinity to a tumor antigen (e.g., a mammalian tumor antigen, such as a human tumor antigen). For example, in some embodiments, the extracellular antigen-binding domain of the CAR (embodied, for example, in a human scFv or an analog thereof) binds to a particular tumor antigen with a dissociation constant (K_{d}) of about 1×10⁻⁵ M or less. In certain embodiments, the K_{d} is about 5×10⁻⁶ M or less, about 1×10⁻⁶ M or less, about 5×10⁻⁷ M or less, about 1×10⁻⁷ M or less, about 5×10⁻⁸ M or less, about 1×10⁻⁸ M or less, about 5×10⁻⁹ or less, about 4×10⁻⁹ or less, about 3×10⁻⁹ or less, about 2×10⁻⁹ or less, or about 1×10⁻⁹ M or less. In certain non-limiting embodiments, the K_{d} is from about 3×10⁻⁹ M or less. In certain non-limiting embodiments, the K_{d} is from about 3×10⁻⁹ to about 2×10⁻⁷.

Binding of the extracellular antigen-binding domain (embodiment, for example, in a human scFv or an analog thereof) of a presently disclosed tumor antigen-targeted CAR can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or a scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the extracellular antigen-binding domain of the tumor antigen-targeted CAR is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet). In certain embodiments, the human scFv of a presently disclosed tumor antigen-targeted CAR is labeled with GFP.

In some embodiments, the extracellular antigen-binding domain of the expressed CAR binds to tumor antigen that is expressed by a tumor cell. In some embodiments, the extracellular antigen-binding domain of the expressed CAR binds to tumor antigen that is expressed on the surface of a tumor cell. In some embodiments, the extracellular antigen-binding domain of the expressed CAR binds to tumor antigen that is expressed on the surface of a tumor cell in combination with an MHC protein. In some embodiments, the MHC protein is a MHC class I protein. In some embodiments, the MHC Class I protein is an HLA-A, HLA-B, or HLA-C molecules. In some embodiments, the extracellular antigen-binding domain of the expressed CAR binds to tumor antigen that is expressed on the surface of a tumor cell not in combination with an MHC protein.

In some embodiments, the extracellular antigen-binding domain of the expressed CAR binds to tumor antigen selected from among 5T4, alpha 5β1-integrin, 707-AP, A33, AFP, ART-4, B7H4, BAGE, Bcl-2, β-catenin, BCMA, Bcr-abl, MN/C IX antibody, CA125, CA19-9, CAMEL, CAP-1, CASP-8, CD4, CD5, CD19, CD20, CD21, CD22, CD25, CDC27/m, CD33, CD37, CD45, CD52, CD56, CD80, CD123, CDK4/m, CEA, c-Met, CS-1, CT, Cyp-B, cyclin B1, DAGE, DAM, EBNA, EGFR, ErbB3, ELF2M, EMMPRIN, EpCam, ephrinB2, estrogen receptor, ETV6-AML1, FAP, ferritin, folate-binding protein, GAGE, G250, GD-2, GM2, GnT-V, gp75, gp100 (Pmel 17), HAGE, HER-2/neu, HLA-A*0201-R170I, HPV E6, HPV E7, Ki-67, HSP70-2M, HST-2, hTERT (or hTRT), iCE, IGF-1R, IL-2R, IL-5, KIAA0205, LAGE, LDLR/FUT, LRP, MAGE, MART, MART-1/melan-A, MART-2/Ski, MC1R, mesothelin, MUC, MUC16, MUM-1-B, myc, MUM-2, MUM-3, NA88-A, NYESO-1, NY-Eso-B, p53, proteinase-3, p190 minor bcr-abl, Pml/RARα, PRAME, progesterone receptor, PSA, PSCA, PSM, PSMA, ras, RAGE, RU1 or RU2, RORI, SART-1 or SART-3, survivin, TEL/AML1, TGFβ, TPI/m, TRP-1, TRP-2, TRP-2/INT2, tenascin, TSTA tyrosinase, VEGF, and WT1. In certain embodiments, the extracellular antigen-binding domain of the expressed CAR binds to tumor antigen selected from among BCMA, CD19, mesothelin, MUC16, PSCA, WT1, and PRAME. Exemplary extracellular antigen-binding domains and methods of generating such domains and associated CARs are described in, e.g., WO2016/191246, WO2017/023859, WO2015/188141, WO2015/070061, WO2012/135854, WO2014/055668, which are incorporated by reference in their entirety, including the sequence listings provided therein.

In some embodiments, the extracellular antigen-binding domain of the expressed CAR binds to a CD19 tumor antigen. In some embodiments, the extracellular antigen-binding domain of the expressed CAR binds to a CD19 tumor antigen presented in the context of an MHC molecule. In some embodiments, the extracellular antigen-binding domain of the expressed CAR binds to a CD19 tumor antigen presented in the context of an HLA-A2 molecule.

In some embodiments, the extracellular antigen-binding domain of the expressed CAR binds to a “preferentially expressed antigen in melanoma” (PRAME) tumor antigen. In some embodiments, the extracellular antigen-binding domain of the expressed CAR binds to a PRAME tumor antigen presented in the context of an MHC molecule. In some embodiments, the extracellular antigen-binding domain of the expressed CAR binds to a PRAME tumor antigen presented in the context of an HLA-A2 molecule.

In some embodiments, extracellular antigen-binding domain of the expressed CAR binds to a WT1 (Wilm's tumor protein 1) tumor antigen. In some embodiments, the extracellular antigen-binding domain of the expressed CAR binds to a WT1 tumor antigen presented in the context of an MHC molecule. In some embodiments, the extracellular antigen-binding domain binds to a WT1 tumor antigen presented in the context of an HLA-A2 molecule.

In some embodiments, extracellular antigen-binding domain of the expressed CAR binds to a MUC16 tumor antigen. In some embodiments, the extracellular antigen-binding domain of the expressed CAR binds to a MUC16 tumor antigen presented in the context of an MHC molecule. In some embodiments, the extracellular antigen-binding domain binds to a MUC16 tumor antigen presented in the context of an HLA-A2 molecule.

In some embodiments, extracellular antigen-binding domain of the expressed CAR binds to a mesothelin tumor antigen. In some embodiments, the extracellular antigen-binding domain of the expressed CAR binds to a mesothelin tumor antigen presented in the context of an MHC molecule. In some embodiments, the extracellular antigen-binding domain binds to a mesothelin tumor antigen presented in the context of an HLA-A2 molecule.

In some embodiments, extracellular antigen-binding domain of the expressed CAR binds to a BCMA (B-cell maturation antigen) tumor antigen. In some embodiments, the extracellular antigen-binding domain of the expressed CAR binds to a BCMA tumor antigen presented in the context of an MHC molecule. In some embodiments, the extracellular antigen-binding domain binds to a BCMA tumor antigen presented in the context of an HLA-A2 molecule.

In some embodiments, extracellular antigen-binding domain of the expressed CAR binds to a PSCA (prostate stem cell antigen) tumor antigen. In some embodiments, the extracellular antigen-binding domain of the expressed CAR binds to a PSCA tumor antigen presented in the context of an MHC molecule. In some embodiments, the extracellular antigen-binding domain binds to a PSCA tumor antigen presented in the context of an HLA-A2 molecule.

In certain embodiments, the extracellular antigen-binding domain (e.g., human scFv) comprises a heavy chain variable region and a light chain variable region, optionally linked with a linker sequence, for example a linker peptide (e.g., SEQ ID NO: 1), between the heavy chain variable region and the light chain variable region. In certain embodiments, the extracellular antigen-binding domain is a human scFv-Fc fusion protein or full length human IgG with V_{H} and V_{L} regions.

In certain embodiments, the extracellular antigen-binding domain comprises a human scFv that binds to a CD19 antigen. In some embodiments, the scFv comprises a polypeptide having an amino acid sequence of SEQ ID NO: 3.

(SEQ ID NO: 3)
EVKLQQSGAELVRPGSSVKISCKASGYAFSSYWMNWVKQR
PGQGLEWIGQIYPGDGDTNYNGKFKGQATLTADKSSSTAY
MQLSGLTSEDSAVYFCARKTISSVVDFYFDYWGQGTTVTV
SSGGGGSGGGGSGGGGSDIELTQSPKFMSTSVGDRVSVTC
KASQNVGTNVAWYQQKPGQSPKPLIYSATYRNSGVPDRFT
GSGSGTDFTLTITNVQSKDLADYFCQQYNRYPYTSGGGTK
LEIKR.

In some embodiments, the scFv comprises a polypeptide having an amino acid sequence of SEQ ID NO: 4.

(SEQ ID NO: 4)
MALPVTALLLPLALLLHAEVKLQQSGAELVRPGSSVKISC
KASGYAFSSYWMNWVKQRPGQGLEWIGQIYPGDGDTNYNG
KFKGQATLTADKSSSTAYMQLSGLTSEDSAVYFCARKTIS
SVVDFYFDYWGQGTTVTVSSGGGGSGGGGSGGGGSDIELT
QSPKFMSTSVGDRVSVTCKASQNVGTNVAWYQQKPGQSPK
PLIYSATYRNSGVPDRFTGSGSGTDFTLTITNVQSKDLAD
YFCQQYNRYPYTSGGGTKLEIKR

In some embodiments, the scFv comprises a polypeptide having an amino acid sequence that is at least 80%, at least 85%, at least 90%, or at least 95% identical to SEQ ID NO: 3 or SEQ ID NO: 4. For example, the scFv comprises a polypeptide having an amino acid sequence that is about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 3 or SEQ ID NO: 4.

In some embodiments, the scFv is encoded by a nucleic acid having a nucleic acid sequence of SEQ ID NO: 5.

(SEQ ID NO: 5)
GAGGTGAAGCTGCAGCAGTCTGGGGCTGAGCTGGTGAGGC
CTGGGTCCTCAGTGAAGATTTCCTGCAAGGCTTCTGGCTA
TGCATTCAGTAGCTACTGGATGAACTGGGTGAAGCAGAGG
CCTGGACAGGGTCTTGAGTGGATTGGACAGATTTATCCTG
GAGATGGTGATACTAACTACAATGGAAAGTTCAAGGGTCA
AGCCACACTGACTGCAGACAAATCCTCCAGCACAGCCTAC
ATGCAGCTCAGCGGCCTAACATCTGAGGACTCTGCGGTCT
ATTTCTGTGCAAGAAAGACCATTAGTTCGGTAGTAGATTT
CTACTTTGACTACTGGGGCCAAGGGACCACGGTCACCGTC
TCCTCAGGTGGAGGTGGATCAGGTGGAGGTGGATCTGGTG
GAGGTGGATCTGACATTGAGCTCACCCAGTCTCCAAAATT
CATGTCCACATCAGTAGGAGACAGGGTCAGCGTCACCTGC
AAGGCCAGTCAGAATGTGGGTACTAATGTAGCCTGGTATC
AACAGAAACCAGGACAATCTCCTAAACCACTGATTTACTC
GGCAACCTACCGGAACAGTGGAGTCCCTGATCGCTTCACA
GGCAGTGGATCTGGGACAGATTTCACTCTCACCATCACTA
ACGTGCAGTCTAAAGACTTGGCAGACTATTTCTGTCAACA
ATATAACAGGTATCCGTACACGTCCGGAGGGGGGACCAAG
CTGGAGATCAAACGGGCGGCCGCA.

In some embodiments, the scFv is encoded by a nucleic acid having a nucleic acid sequence of SEQ ID NO: 6.

(SEQ ID NO: 6)
ATGGCTCTCCCAGTGACTGCCCTACTGCTTCCCCTAGCGC
TTCTCCTGCATGCAGAGGTGAAGCTGCAGCAGTCTGGGGC
TGAGCTGGTGAGGCCTGGGTCCTCAGTGAAGATTTCCTGC
AAGGCTTCTGGCTATGCATTCAGTAGCTACTGGATGAACT
GGGTGAAGCAGAGGCCTGGACAGGGTCTTGAGTGGATTGG
ACAGATTTATCCTGGAGATGGTGATACTAACTACAATGGA
AAGTTCAAGGGTCAAGCCACACTGACTGCAGACAAATCCT
CCAGCACAGCCTACATGCAGCTCAGCGGCCTAACATCTGA
GGACTCTGCGGTCTATTTCTGTGCAAGAAAGACCATTAGT
TCGGTAGTAGATTTCTACTTTGACTACTGGGGCCAAGGGA
CCACGGTCACCGTCTCCTCAGGTGGAGGTGGATCAGGTGG
AGGTGGATCTGGTGGAGGTGGATCTGACATTGAGCTCACC
CAGTCTCCAAAATTCATGTCCACATCAGTAGGAGACAGGG
TCAGCGTCACCTGCAAGGCCAGTCAGAATGTGGGTACTAA
TGTAGCCTGGTATCAACAGAAACCAGGACAATCTCCTAAA
CCACTGATTTACTCGGCAACCTACCGGAACAGTGGAGTCC
CTGATCGCTTCACAGGCAGTGGATCTGGGACAGATTTCAC
TCTCACCATCACTAACGTGCAGTCTAAAGACTTGGCAGAC
TATTTCTGTCAACAATATAACAGGTATCCGTACACGTCCG
GAGGGGGGACCAAGCTGGAGATCAAACGG

In some embodiments, the scFv is encoded by a nucleic acid having a nucleic acid sequence that is at least 80%, at least 85%, at least 90%, or at least 95% identical to SEQ ID NO: 5 or SEQ ID NO: 6. In some embodiments, the scFv is encoded by a nucleic acid having a nucleic acid sequence of SEQ ID NO: 5 or SEQ ID NO: 6. In some embodiments, the scFv is encoded by a nucleic acid having a nucleic acid sequence that is about 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 5 or SEQ ID NO: 6.

In certain non-limiting embodiments, an extracellular antigen-binding domain of the presently disclosed CAR can comprise a linker connecting the heavy chain variable region and light chain variable region of the extracellular antigen-binding domain. As used herein, the term “linker” refers to a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker comprises amino acids having the sequence set forth in SEQ ID NO: 1, SEQ ID NO: 33, or SEQ ID NO: 34. In certain embodiments, the nucleotide sequence encoding the amino acid sequence of SEQ ID NO: 1 is set forth in SEQ ID NO: 2.

In addition, the extracellular antigen-binding domain can comprise a leader or a signal peptide that directs the nascent protein into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway.

In certain embodiments, the signal peptide is covalently joined to the N-terminus of the extracellular antigen-binding domain. In certain embodiments, the signal peptide comprises a CD8 signal polypeptide comprising amino acids having the sequence set forth in SEQ ID NO: 7 as provided below: MALPVTALLLPLALLLHAARP (SEQ ID NO: 7).

The nucleotide sequence encoding the amino acid sequence of SEQ ID NO: 7 is set forth in SEQ ID NO: 8, which is provided below: atggccctgccagtaacggctctgctgctgccacttgctctgctcctccatgcagccaggcct (SEQ ID NO: 8).

In certain embodiments, the signal peptide comprises a CD8 signal polypeptide comprising amino acids having the sequence set forth in SEQ ID NO: 9 as provided below:

(SEQ ID NO: 9)
MALPVTALLLPLALLLHA.

The nucleotide sequence encoding the amino acid sequence of SEQ ID NO: 9 is set forth in SEQ ID NO: 10, which is provided below:

(SEQ ID NO: 10)
ATGGCTCTCCCAGTGACTGCCCTACTGCTTCCCCTAGCGC
TTCTCCTGCATGCA.

Transmembrane Domain of a CAR. In certain non-limiting embodiments, the transmembrane domain of the CAR comprises a hydrophobic alpha helix that spans at least a portion of the membrane. Different transmembrane domains result in different receptor stability. After antigen recognition, receptors cluster and a signal is transmitted to the cell. In accordance with the presently disclosed subject matter, the transmembrane domain of the CAR can comprise a CD8 polypeptide, a CD28 polypeptide, a CD3ζ polypeptide, a CD4 polypeptide, a 4-1BB polypeptide, an OX40 polypeptide, an ICOS polypeptide, a CTLA-4 polypeptide, a PD-1 polypeptide, a LAG-3 polypeptide, a 2B4 polypeptide, a BTLA polypeptide, a synthetic peptide (e.g., a transmembrane peptide not based on a protein associated with the immune response), or a combination thereof.

In certain embodiments, the transmembrane domain of a presently disclosed CAR comprises a CD28 polypeptide. The CD28 polypeptide can have an amino acid sequence that is at least about 85%, about 90%, about 95%, about 96%, about 97%, about 98%, about 99% or 100% homologous to the sequence having a NCBI Reference No: PI0747 or NP006130 (SEQ ID NO: 11), or fragments thereof, and/or may optionally comprise up to one or up to two or up to three conservative amino acid substitutions. In certain embodiments, the CD28 polypeptide can have an amino acid sequence that is a consecutive portion of SEQ ID NO: 11 which is at least 20, or at least 30, or at least 40, or at least 50, and up to 220 amino acids in length. Alternatively or additionally, in non-limiting various embodiments, the CD28 polypeptide has an amino acid sequence of amino acids 1 to 220, 1 to 50, 50 to 100, 100 to 150, 114 to 220, 150 to 200, or 200 to 220 of SEQ ID NO: 11. In certain embodiments, the CAR of the presently disclosed comprises a transmembrane domain comprising a CD28 polypeptide, and an intracellular domain comprising a co-stimulatory signaling region that comprises a CD28 polypeptide. In certain embodiments, the CD28 polypeptide comprised in the transmembrane domain and the intracellular domain has an amino acid sequence of amino acids 114 to 220 of SEQ ID NO: 11.

SEQ ID NO: 11 is provided below:

(SEQ ID NO: 11)
MLRLLLALNLFPSIQVTGNKILVKQSPMLVAYDNAVNLSC
KYSYNLFSREFRASLHKGLDSAVEVCVVYGNYSQQLQVYS
KTGFNCDGKLGNESVTFYLQNLYVNQTDIYFCKIEVMYPP
PYLDNEKSNGTIIHVKGKHLCPSPLFPGPSKPFWVLVVVG
GVLACYSLLVTVAFIIFWVRSKRSRLLHSDYMNMTPRRPG
PTRKHYQPYAPPRDFAAYRS

In accordance with the presently disclosed subject matter, a “CD28 nucleic acid molecule” refers to a polynucleotide encoding a CD28 polypeptide. In certain embodiments, the CD28 nucleic acid molecule encoding the CD28 polypeptide comprised in the transmembrane domain and the intracellular domain (e.g., the co-stimulatory signaling region) of the presently disclosed CAR (amino acids 114 to 220 of SEQ ID NO: 11) comprises nucleic acids having the sequence set forth in SEQ ID NO: 12 as provided below.

(SEQ ID NO: 12)
attgaagttatgtatcctcctccttacctagacaatgaga
agagcaatggaaccattatccatgtgaaagggaaacacct
ttgtccaagtcccctatttcccggaccttctaagcccttt
tgggtgctggtggtggttggtggagtcctggcttgctata
gcttgctagtaacagtggcctttattattttctgggtgag
gagtaagaggagcaggctcctgcacagtgactacatgaac
atgactccccgccgccccgggcccacccgcaagcattacc
agccctatgccccaccacgcgacttcgcagcctatcgctc
c

In certain embodiments, the transmembrane domain comprises a CD8 polypeptide. The CD8 polypeptide can have an amino acid sequence that is at least about 85%, about 90%, about 95%, about 96%, about 97%, about 98%, about 99% or about 100%) homologous to SEQ ID NO: 13 (homology herein may be determined using standard software such as BLAST or FASTA) as provided below, or fragments thereof, and/or may optionally comprise up to one or up to two or up to three conservative amino acid substitutions. In certain embodiments, the CD8 polypeptide can have an amino acid sequence that is a consecutive portion of SEQ ID NO: 13 which is at least 20, or at least 30, or at least 40, or at least 50, and up to 235 amino acids in length. Alternatively or additionally, in various embodiments, the CD8 polypeptide has an amino acid sequence of amino acids 1 to 235, 1 to 50, 50 to 100, 100 to 150, 150 to 200, or 200 to 235 of SEQ ID NO: 13.

(SEQ ID NO: 13)
MALPVTALLLPLALLLHAARPSQFRVSPLDRTWNLGETVE
LKCQVLLSNP
TSGCSWLFQPRGAAASPTFLLYLSQNKPKAAEGLDTQRFS
GKRLGDTFVLTLSDFRRENEGYYFCSALSNSIMYFSHFVP
VFLPAKPTTTPAPRPPTPAPTIASQPLSLRPEACRPAAGG
AVHTRGLDFACDIYIWAPLAGTCGVLLLSLVITLYCNHRN
RRRVCKCPRPVVKSGDKPSLSARYV

In certain embodiments, the transmembrane domain comprises a CD8 polypeptide comprising amino acids having the sequence set forth in SEQ ID NO: 14 as provided below:

(SEQ ID NO: 14)
PTTTPAPRPPTPAPTIASQPLSLRPEACRPAAGGAVHTRG
LDFACDIYIWAPLAGTCGVLLLSLVITLYCN

In accordance with the presently disclosed subject matter, a “CD8 nucleic acid molecule” refers to a polynucleotide encoding a CD8 polypeptide. In certain embodiments, the CD8 nucleic acid molecule encoding the CD8 polypeptide comprised in the transmembrane domain of the presently disclosed CAR (SEQ ID NO: 14) comprises nucleic acids having the sequence set forth in SEQ ID NO: 15 as provided below.

(SEQ ID NO: 15)
CCCACCACGACGCCAGCGCCGCGACCACCAACCCCGGCGC
CCACGATCGCGTCGCAGCCCCTGTCCCTGCGCCCAGAGGC
GTGCCGGCCAGCGGCGGGGGGCGCAGTGCACACGAGGGGG
CTGGACTTCGCCTGTGATATCTACATCTGGGCGCCCCTGG
CCGGGACTTGTGGGGTCCTTCTCCTGTCACTGGTTATCAC
CCTTTACTGCAAC

In certain non-limiting embodiments, a CAR can also comprise a spacer region that links the extracellular antigen-binding domain to the transmembrane domain. The spacer region can be flexible enough to allow the antigen-binding domain to orient in different directions to facilitate antigen recognition while preserving the activating activity of the CAR. In certain non-limiting embodiments, the spacer region can be the hinge region from IgG1, the CH₂CH₃ region of immunoglobulin and portions of CD3, a portion of a CD28 polypeptide (e.g., SEQ ID NO: 11), a portion of a CD8 polypeptide (e.g., SEQ ID NO: 13), a variation of any of the foregoing which is at least about 80%, at least about 85%>, at least about 90%, or at least about 95% homologous thereto, or a synthetic spacer sequence. In certain non-limiting embodiments, the spacer region may have a length between about 1-50 (e.g., 5-25, 10-30, or 30-50) amino acids.

Intracellular Domain of a CAR. In certain non-limiting embodiments, an intracellular domain of the CAR can comprise a CD3ζ polypeptide, which can activate or stimulate a cell (e.g., a cell of the lymphoid lineage, e.g., a T cell). CD3ζ comprises 3 ITAMs, and transmits an activation signal to the cell (e.g., a cell of the lymphoid lineage, e.g., a T cell) after antigen is bound. The CD3ζ polypeptide can have an amino acid sequence that is at least about 85%, about 90%, about 95%, about 96%, about 97%, about 98%, about 99% or about 100% homologous to the sequence having a NCBI Reference No: NP_932170 (SEQ ID NO: 16), or fragments thereof, and/or may optionally comprise up to one or up to two or up to three conservative amino acid substitutions. In certain embodiments, the CD3ζ polypeptide can have an amino acid sequence that is a consecutive portion of SEQ ID NO: 17 which is at least 20, or at least 30, or at least 40, or at least 50, and up to 164 amino acids in length. Alternatively or additionally, in various embodiments, the CD3ζ polypeptide has an amino acid sequence of amino acids 1 to 164, 1 to 50, 50 to 100, 100 to 150, or 150 to 164 of SEQ ID NO: 17. In certain embodiments, the CD3ζ polypeptide has an amino acid sequence of amino acids 52 to 164 of SEQ ID NO: 17.

SEQ ID NO: 17 is provided below:

(SEQ ID NO: 17)
MKWKALFTAAILQAQLPITEAQSFGLLDPKLCYLLDGILFIYGVILTALF
LRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATK
DTYDALHMQALPPR

In certain embodiments, the CD3ζ polypeptide has the amino acid sequence set forth in SE ID NO: 18, which is provided below:

(SEQ ID NO: 18)
RVKFSRSAEPPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

In certain embodiments, the CD3ζ polypeptide has the amino acid sequence set forth in SEQ ID NO: 19, which is provided below:

(SEQ ID NO: 19)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

In accordance with the presently disclosed subject matter, a “CD3ζ nucleic acid molecule” refers to a polynucleotide encoding a CD3ζ polypeptide. In certain embodiments, the CD3ζ nucleic acid molecule encoding the CD3ζ polypeptide (SEQ ID NO: 18) comprised in the intracellular domain of the presently disclosed CAR comprises a nucleotide sequence as set forth in SEQ ID NO: 20 as provided below.

(SEQ ID NO: 20)
agagtgaagttcagcaggagcgcagagccccccgcgtaccagcagggcca
gaaccagctctataacgagctcaatctaggacgaagagaggagtacgatg
ttttggacaagagacgtggccgggaccctgagatggggggaaagccgaga
aggaagaaccctcaggaaggcctgtacaatgaactgcagaaagataagat
ggcggaggcctacagtgagattgggatgaaaggcgagcgccggaggggca
aggggcacgatggcctttaccagggtctcagtacagccaccaaggacacc
tacgacgcccttcacatgcaggccctgccccctcgcg

In certain embodiments, the CD3ζ nucleic acid molecule encoding the CD3ζ polypeptide (SEQ ID NO: 19) comprised in the intracellular domain of the presently disclosed CAR comprises a nucleotide sequence as set forth in SEQ ID NO: 21 as provided below.

(SEQ ID NO: 21)
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTACAATGAACTGCAGAAAGATAAGAT
GGCGGAGGCCTACAGTGAGATTGGGATGAAAGGCGAGCGCCGGAGGGGCA
AGGGGCACGATGGCCTTTACCAGGGTCTCAGTACAGCCACCAAGGACACC
TACGACGCCCTTCACATGCAGGCCCTGCCCCCTCGCTAA

In certain non-limiting embodiments, an intracellular domain of the CAR further comprises at least one signaling region. The at least one signaling region can include a CD28 polypeptide, a 4-1BB polypeptide, an OX40 polypeptide, an ICOS polypeptide, a DAP-10 polypeptide, a PD-1 polypeptide, a CTLA-4 polypeptide, a LAG-3 polypeptide, a 2B4 polypeptide, a BTLA polypeptide, a synthetic peptide (not based on a protein associated with the immune response), or a combination thereof.

In certain embodiments, the signaling region is a co-stimulatory signaling region.

In certain embodiments, the co-stimulatory signaling region comprises at least one co-stimulatory molecule, which can provide optimal lymphocyte activation. As used herein, “co-stimulatory molecules” refer to cell surface molecules other than antigen receptors or their ligands that are required for an efficient response of lymphocytes to antigen. The at least one co-stimulatory signaling region can include a CD28 polypeptide, a 4-1BB polypeptide, an OX40 polypeptide, an ICOS polypeptide, a DAP-10 polypeptide, or a combination thereof. The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to its CAR molecule. Co-stimulatory ligands, include, but are not limited to CD80, CD86, CD70, OX40L, 4-1BBL, CD48, TNFRSF14, and PD-L1. As one example, a 4-1BB ligand (i.e., 4-1BBL) may bind to 4-1BB (also known as “CD 137”) for providing an intracellular signal that in combination with a CAR signal induces an effector cell function of the CAR⁺ T cell. CARs comprising an intracellular domain that comprises a co-stimulatory signaling region comprising 4-1BB, ICOS or DAP-10 are disclosed in U.S. Pat. No. 7,446,190, which is herein incorporated by reference in its entirety. In certain embodiments, the intracellular domain of the CAR comprises a co-stimulatory signaling region that comprises a CD28 polypeptide. In certain embodiments, the intracellular domain of the CAR comprises a co-stimulatory signaling region that comprises two co-stimulatory molecules: CD28 and 4-1BB or CD28 and OX40.

The 4-1BB polypeptide can have an amino acid sequence that is at least about 85%, about 90%, about 95%, about 96%, about 97%, about 98%, about 99% or 100% homologous to the sequence having a NCBI Reference No: P41273 or NP_001552 (SEQ ID NO: 22) or fragments thereof, and/or may optionally comprise up to one or up to two or up to three conservative amino acid substitutions.

SEQ ID NO: 22 is provided below:

(SEQ ID NO: 22)
MGNSCYNIVATLLLVLNFERTRSLQDPCSNCPAGTFCDNNRNQICSPCPP
NSFSSAGGQRTCDICRQCKGVFRTRKECSSTSNAECDCTPGFHCLGAGCS
MCEQDCKQGQELTKKGCKDCCFGTFNDQKRGICRPWTNCSLDGKSVLVNG
TKERDVVCGPSPADLSPGASSVTPPAPAREPGHSPQIISFFLALTSTALL
FLLFFLTLRFSVVKRGRKKLLYIFKQPFMRPVQTTQEEDGCSCRFPEEEE
GGCEL

In certain embodiments, the 4-1BB co-stimulatory domain has the amino acid sequence set forth in SEQ ID NO: 23, which is provided below:

(SEQ ID NO: 23)
KRGRKKLLYIFKQPFMRPVQTTQEEDGCSCRFPEEEEGGCEL

In accordance with the presently disclosed subject matter, a “4-1BB nucleic acid molecule” refers to a polynucleotide encoding a 4-1BB polypeptide. In certain embodiments, the 4-1BB nucleic acid molecule encoding the 4-1BB polypeptide (SEQ ID NO: 23) comprised in the intracellular domain of the presently disclosed CAR comprises a nucleotide sequence as set forth in SEQ ID NO: 24 as provided below.

(SEQ ID NO: 24)
AAACGGGGCAGAAAGAAGCTCCTGTATATATTCAAACAACCATTTATGAG
ACCAGTACAAACTACTCAAGAGGAAGATGGCTGTAGCTGCCGATTTCCAG
AAGAAGAAGAAGGAGGATGTGAACTG

An OX40 polypeptide can have an amino acid sequence that is at least about 85%, about 90%, about 95%, about 96%, about 97%, about 98%, about 99% or 100% homologous to the sequence having a NCBI Reference No: P43489 or NP 003318 (SEQ ID NO: 25), or fragments thereof, and/or may optionally comprise up to one or up to two or up to three conservative amino acid substitutions.

SEQ ID NO: 25 is provided below:

(SEQ ID NO: 25)
MCVGARRLGRGPCAALLLLGLGLSTVTGLHCVGDTYPSNDRCCHECRPGN
GMVSRCSRSQNTVCRPCGPGFYNDVVSSKPCKPCTWCNLRSGSERKQLCT
ATQDTVCRCRAGTQPLDSYKPGVDCAPCPPGHFSPGDNQACKPWTNCTLA
GKHTLQPASNSSDAICEDRDPPATQPQETQGPPARPITVQPTEAWPRTSQ
GPSTRPVEVPGGRAVAAILGLGLVLGLLGPLAILLALYLLRRDQRLPPDA
HKPPGGGSFRTPIQEEQADAHSTLAKI

In accordance with the presently disclosed subject matter, an “OX40 nucleic acid molecule” refers to a polynucleotide encoding an OX40 polypeptide.

An ICOS polypeptide can have an amino acid sequence that is at least about 85%, about 90%, about 95%, about 96%, about 97%, about 98%, about 99% or 100% homologous to the sequence having a NCBI Reference No: NP_036224 (SEQ ID NO: 26) or fragments thereof, and/or may optionally comprise up to one or up to two or up to three conservative amino acid substitutions.

SEQ ID NO: 26 is provided below:

(SEQ ID NO: 26)
MKSGLWYFFLFCLRIKVLTGEINGSANYEMFIFHNGGVQILCKYPDIVQQ
FKMQLLKGGQILCDLTKTKGSGNTVSIKSLKFCHSQLSNNSVSFFLYNLD
HSHANYYFCNLSIFDPPPFKVTLTGGYLHIYESQLCCQLKFWLPIGCAAF
VVVCILGCILICWLTKKKYSSSVHDPNGEYMFMRAVNTAKKSRLTDVTL

In accordance with the presently disclosed subject matter, an “ICOS nucleic acid molecule” refers to a polynucleotide encoding an ICOS polypeptide.

In accordance with the presently disclosed subject matter, a CTLA-4 polypeptide can have an amino acid sequence that is at least about 85%, about 90%, about 95%, about 96%, about 97%, about 98%, about 99% or about 100% homologous to UniProtKB/Swiss-Prot Ref. No.: P16410.3 (SEQ ID NO: 27) (homology herein may be determined using standard software such as BLAST or FASTA) or fragments thereof, and/or may optionally comprise up to one or up to two or up to three conservative amino acid substitutions.

SEQ ID NO: 27 is provided below:

(SEQ ID NO: 27)
MACLGFQRHKAQLNLATRTWPCTLLFFLLFIPVFCKAMHVAQPAVVLASS
RGIASFVCEYASPGKATEVRVTVLRQADSQVTEVCAATYMMGNELTFLDD
SICTGTSSGNQVNLTIQGLRAMDTGLYICKVELMYPPPYYLGIGNGTQIY
VIDPEPCPDSDFLLWILAAVSSGLFFYSFLLTAVSLSKMLKKRSPLTTGV
YVKMPPTEPECEKQFQPYFIPIN

In accordance with the presently disclosed subject matter, a “CTLA-4 nucleic acid molecule” refers to a polynucleotide encoding a CTLA-4 polypeptide.

In accordance with the presently disclosed subject matter, a PD-1 polypeptide can have an amino acid sequence that is at least about 85%, about 90%, about 95%, about 96%, about 9700, about 98%, about 99% or about 100% homologous to NCBI Reference No: NP_005009.2 (SEQ TD NO: 28) or fragments thereof, and/or may optionally comprise up to one or up to two or up to three conservative amino acid substitutions.

SEQ ID NO: 28 is provided below:

(SEQ ID NO: 28)
MQIPQAPWPVVWAVLQLGWRPGWFLDSPDRPWNPPTFSPALLVVTEGDNA
TFTCSFSNTSESFVLNWYRMSPSNQTDKLAAFPEDRSQPGQDCRFRVTQL
PNGRDFHMSVVRVGVVGGLLGSLVLLVWVLAVICSRAARGTIGARRTGQP
LKEDPSAVPVFSVDYGELDFQWREKTPEPPVPCVPEQTEYATIVFPSGMG
TSSPARRGSADGPRSAQPLRPEDGHCSWPL

In accordance with the presently disclosed subject matter, a “PD-1 nucleic acid molecule” refers to a polynucleotide encoding a PD-1 polypeptide.

In accordance with the presently disclosed subject matter, a LAG-3 polypeptide can have an amino acid sequence that is at least about 850%, about 900%, about 950%, about 96%, about 97%, about 98%, about 99% or about 100% homologous to UniProtKB/Swiss-Prot Ref. No.: P18627.5 (SEQ ID NO: 29) or fragments thereof, and/or may optionally comprise up to one or up to two or up to three conservative amino acid substitutions.

SEQ ID NO: 29 is provided below:

(SEQ ID NO: 29)
MWEAQFLGLLFLQPLWVAPVKPLQPGAEVPVVWAQEGAPAQLPCSPTIPL
QDLSLLRRAGVTWQHQPDSGPPAAAPGHPLAPGPHPAAPSSWGPRPRRYT
VLSVGPGGLRSGRLPLQPRVQLDERGRQRGDFSLWLRPARRADAGEYRAA
VHLRDRALSCRLRLRLGQASMTASPPGSLRASDWVILNCSFSRPDRPASV
HWFRNRGQGRVPVRESPHHHLAESFLFLPQVSPMDSGPWGCILTYRDGFN
VSIMYNLTVLGLEPPTPLTVYAGAGSRVGLPCRLPAGVGTRSFLTAKWTP
PGGGPDLLVTGDNGDFTLRLEDVSQAQAGTYTCHIHLQEQQLNATVTLAI
ITVTPKSFGSPGSLGKLLCEVTPVSGQERFVWSSLDTPSQRSFSGPWLEA
QEAQLLSQPWQCQLYQGERLLGAAVYFTELSSPGAQRSGRAPGALPAGHL
LLFLILGVLSLLLLVTGAFGFHLWRRQWRPRRFSALEQGIHPPQAQSKIE
ELEQEPEPEPEPEPEPEPEPEPEQL

In accordance with the presently disclosed subject matter, a “LAG-3 nucleic acid molecule” refers to a polynucleotide encoding a LAG-3 polypeptide.

In accordance with the presently disclosed subject matter, a 2B4 polypeptide can have an amino acid sequence that is at least about 85%, about 90%, about 95%, about 96%, about 97%, about 98%, about 99% or about 100% homologous to UniProtKB/Swiss-Prot Ref No.: Q9BZW8.2 (SEQ ID NO: 30) or fragments thereof, and/or may optionally comprise up to one or up to two or up to three conservative amino acid substitutions.

SEQ ID NO: 30 is provided below:

(SEQ ID NO: 30)
MLGQVVTLILLLLLKVYQGKGCQGSADHVVSISGVPLQLQPNSIQTKVDS
IAWKKLLPSQNGFHHILKWENGSLPSNTSNDRFSFIVKNLSLLIKAAQQQ
DSGLYCLEVTSISGKVQTATFQVFVFESLLPDKVEKPRLQGQGKILDRGR
CQVALSCLVSRDGNVSYAWYRGSKLIQTAGNLTYLDEEVDINGTHTYTCN
VSNPVSWESHTLNLTQDCQNAHQEFRFWPFLVIIVILSALFLGTLACFCV
WRRKRKEKQSETSPKEFLTIYEDVKDLKTRRNHEQEQTFPGGGSTIYSMI
QSQSSAPTSQEPAYTLYSLIQPSRKSGSRKRNHSPSFNSTIYEVIGKSQP
KAQNPARLSRKELENFDVYS

In accordance with the presently disclosed subject matter, a “2B4 nucleic acid molecule” refers to a polynucleotide encoding a 2B4 polypeptide.

In accordance with the presently disclosed subject matter, a BTLA polypeptide can have an amino acid sequence that is at least about 85%>, about 90%, about 95%, about 96%, about 97%, about 98%, about 99% or about 100% homologous to UniProtKB/Swiss-Prot Ref. No.: Q7Z6A9.3 (SEQ ID NO: 31) or fragments thereof, and/or may optionally comprise up to one or up to two or up to three conservative amino acid substitutions.

SEQ ID NO: 31 is provided below:

(SEQ ID NO: 31)
MKTLPAMLGTGKLFWVFFLIPYLDIWNIHGKESCDVQLYIKRQSEHSILA
GDPFELECPVKYCANRPHVTWCKLNGTTCVKLEDRQTSWKEEKNISFFIL
HFEPVLPNDNGSYRCSANFQSNLIESHSTTLYVTDVKSASERPSKDEMAS
RPWLLYRLLPLGGLPLLITTCFCLFCCLRRHQGKQNELSDTAGREINLVD
AHLKSEQTEASTRQNSQVLLSETGIYDNDPDLCFRMQEGSEVYSNPCLEE
NKPGIVYASLNHSVIGPNSRLARNVKEAPTEYASICVRS

In accordance with the presently disclosed subject matter, a “BTLA nucleic acid molecule” refers to a polynucleotide encoding a BTLA polypeptide.

### Exemplary CAR and C825 Antigen-Binding Fragment Constructs

In certain embodiments, the CAR and the anti-DOTA C825 antigen binding fragment are expressed as single polypeptide linked by a self-cleaving linker, such as a P2A linker. In certain embodiments, the CAR and the anti-DOTA C825 antigen binding fragment are expressed as two separate polypeptides.

In certain embodiments, the CAR comprises an extracellular antigen-binding region that comprises a human scFv that specifically binds to a human tumor antigen, a transmembrane domain comprising a CD28 polypeptide and/or a CD8 polypeptide, and an intracellular domain comprising a CD3ζ polypeptide and a co-stimulatory signaling region that comprises a 4-1BB polypeptide. The CAR also comprises a signal peptide or a leader covalently joined to the N-terminus of the extracellular antigen-binding domain. The signal peptide comprises amino acids having the sequence set forth in SEQ ID NO: 7 or SEQ ID NO: 9. In certain embodiments, the human scFv is selected from the group consisting of an anti-BCMA scFv, an anti-CD19 scFv, an anti-mesothelin scFv, an anti-MUC16 scFv, an anti-PSCA scFv, an anti-WT1 scFv, and an anti-PRAME scFv.

In some embodiments, the nucleic acid encoding the CAR and the anti-DOTA C825 antigen binding fragment is operably linked an inducible promoter. In some embodiments, the nucleic acid encoding the CAR and the anti-DOTA C825 antigen binding fragment is operably linked a constitutive promoter. In some embodiments, the nucleic acid encoding the CAR and the nucleic acid encoding and the anti-DOTA C825 antigen binding fragment are operably linked to two separate promoters. In some embodiments, the nucleic acid encoding the CAR is operably linked a constitutive promoter and the anti-DOTA C825 antigen binding fragment is operably linked a constitutive promoter. In some embodiments, the nucleic acid encoding the CAR is operably linked a constitutive promoter and the anti-DOTA C825 antigen binding fragment is operably linked an inducible promoter.

In some embodiments, the inducible promoter is a synthetic Notch promoter that is activatable in a CAR T cell, where the intracellular domain of the CAR contains a transcriptional regulator that is released from the membrane when engagement of the CAR with the tumor antigen induces intramembrane proteolysis (see, e.g., Morsut et al., Cell 164(4): 780-791 (2016). Accordingly, transcription of the anti-DOTA C825 antigen binding fragment is induced upon binding of the engineered immune cell with the tumor antigen.

The presently disclosed subject matter also provides isolated nucleic acid molecules encoding the CAR/anti-DOTA C825 antigen binding fragment constructs described herein or a functional portion thereof. In certain embodiments, the isolated nucleic acid molecule encodes an anti-CD19-targeted CAR comprising a human scFv that specifically binds to a human CD19 polypeptide, a transmembrane domain comprising a CD8 polypeptide, and an intracellular domain comprising a CD3ζ polypeptide and a co-stimulatory signaling region comprising a 4-1BB polypeptide, a P2A self-cleaving peptide, and an anti-DOTA C825 antigen binding fragment provided herein.

In certain embodiments, the isolated nucleic acid molecule encodes an anti-CD19-targeted CAR comprising a human scFv that specifically binds to a human CD19 polypeptide fused to a synthetic Notch transmembrane domain and an intracellular cleavable transcription factor. In certain embodiments, the isolated nucleic acid molecule encodes an anti-DOTA C825 antigen binding fragment inducible by release of the transcription factor of a synthetic Notch system.

In certain embodiments, the isolated nucleic acid molecule encodes an anti-MUC16-targeted CAR comprising a human scFv that specifically binds to a human MUC16 polypeptide, a transmembrane domain comprising a CD8 polypeptide, and an intracellular domain comprising a CD3ζ polypeptide and a co-stimulatory signaling region comprising a 4-1BB polypeptide, a P2A self-cleaving peptide, and an anti-DOTA C825 antigen binding fragment provided herein.

In certain embodiments, the isolated nucleic acid molecule encodes an anti-mesothelin-targeted CAR comprising a human scFv that specifically binds to a human mesothelin polypeptide, a transmembrane domain comprising a CD8 polypeptide, and an intracellular domain comprising a CD3ζ polypeptide and a co-stimulatory signaling region comprising a 4-1BB polypeptide, a P2A self-cleaving peptide, and an anti-DOTA C825 antigen binding fragment provided herein.

In certain embodiments, the isolated nucleic acid molecule encodes an anti-WT1-targeted CAR comprising a human scFv that specifically binds to a human WT1 polypeptide, a transmembrane domain comprising a CD8 polypeptide, and an intracellular domain comprising a CD3ζ polypeptide and a co-stimulatory signaling region comprising a 4-1BB polypeptide, a P2A self-cleaving peptide, and an anti-DOTA C825 antigen binding fragment provided herein.

In certain embodiments, the isolated nucleic acid molecule encodes an anti-PSCA-targeted CAR comprising a human scFv that specifically binds to a human PSCA polypeptide, a transmembrane domain comprising a CD8 polypeptide, and an intracellular domain comprising a CD3ζ polypeptide and a co-stimulatory signaling region comprising a 4-1BB polypeptide, a P2A self-cleaving peptide, and an anti-DOTA C825 antigen binding fragment provided herein.

In certain embodiments, the isolated nucleic acid molecule encodes an anti-BCMA-targeted CAR comprising a human scFv that specifically binds to a human BCMA polypeptide, a transmembrane domain comprising a CD8 polypeptide, and an intracellular domain comprising a CD3ζ polypeptide and a co-stimulatory signaling region comprising a 4-1BB polypeptide, a P2A self-cleaving peptide, and an anti-DOTA C825 antigen binding fragment provided herein.

In certain embodiments, the isolated nucleic acid molecule encodes a functional portion of a presently disclosed CAR constructs. As used herein, the term “functional portion” refers to any portion, part or fragment of a CAR, which portion, part or fragment retains the biological activity of the targeted CAR (the parent CAR). In certain embodiments, an isolated nucleic acid molecule encoding a functional portion of a tumor antigen-targeted CAR can encode a protein comprising, e.g., about 10%, about 20%, about 25%, about 30%, about 35%, about 40%, about 45%, about 50%, about 55%, about 60%, about 65%, about 70%, about 75%, about 80%, about 85%, about 90%, and about 95%, or more of the parent CAR.

### Immune Cells

The presently disclosed subject matter provides engineered immune cells expressing an anti-DOTA C825 antigen binding fragment and a T-cell receptor (e.g., a CAR) or other ligand that comprises an extracellular antigen-binding domain, a transmembrane domain and an intracellular domain, where the extracellular antigen-binding domain specifically binds tumor antigen, including a tumor receptor or ligand, as described above. In certain embodiments immune cells can be transduced with a presently disclosed CAR/anti-DOTA C825 antigen binding fragment constructs such that the cells express the CAR and the anti-DOTA C825 antigen binding fragment.

The engineered immune cells of the presently disclosed subject matter can be cells of the lymphoid lineage or myeloid lineage. The lymphoid lineage, comprising B, T, and natural killer (NK) cells, provides for the production of antibodies, regulation of the cellular immune system, detection of foreign agents in the blood, detection of cells foreign to the host, and the like. Non-limiting examples of immune cells of the lymphoid lineage include T cells, Natural Killer (NK) cells, embryonic stem cells, and pluripotent stem cells (e.g., those from which lymphoid cells may be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, T helper cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), Natural killer T cells, Mucosal associated invariant T cells, and γδ T cells. Cytotoxic T cells (CTL or killer T cells) are a subset of T lymphocytes capable of inducing the death of infected somatic or tumor cells. In certain embodiments, the CAR-expressing T cells express Foxp3 to achieve and maintain a T regulatory phenotype.

Natural killer (NK) cells can be lymphocytes that are part of cell-mediated immunity and act during the innate immune response. NK cells do not require prior activation in order to perform their cytotoxic effect on target cells.

The engineered immune cells of the presently disclosed subject matter can express an extracellular antigen-binding domain (e.g., a human scFv, a Fab that is optionally crosslinked, or a F(ab)₂) that specifically binds to a tumor antigen. In some embodiments, the immune cell is a lymphocyte, such as a T cell, a B cell or a natural killer (NK) cell. In certain embodiments, the engineered immune cell is a T cell. The T cell can be a CD4⁺ T cell or a CD8⁺ T cell. In certain embodiments, the T cell is a CD4⁺ T cell. In certain embodiments, the T cell is a CD8⁺ T cell.

A presently disclosed engineered immune cells can further include at least one recombinant or exogenous co-stimulatory ligand. For example, a presently disclosed engineered immune cells can be further transduced with at least one co-stimulatory ligand, such that the engineered immune cells co-expresses or is induced to co-express the tumor antigen-targeted CAR and the at least one co-stimulatory ligand. The interaction between the tumor antigen-targeted CAR and at least one co-stimulatory ligand provides a non-antigen-specific signal important for full activation of an immune cell (e.g., T cell). Co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD40L (CD40L)/CD 154, CD137L/4-1BBL, TNF-α, CD134L/OX40L/CD252, CD27L/CD70, Fas ligand (FasL), CD30L/CD153, tumor necrosis factor beta (TNFP)/lymphotoxin-alpha (LT-α), lymphotoxin-beta (LT-β), CD257/B cell-activating factor (BAFF)/BLYS/THANK/TALL-1, glucocorticoid-induced TNF Receptor ligand (GITRL), TNF-related apoptosis-inducing ligand (TRAIL), and LIGHT (TNFSF14). The immunoglobulin (Ig) superfamily is a large group of cell surface and soluble proteins that are involved in the recognition, binding, or adhesion processes of cells. These proteins share structural features with immunoglobulins they possess an immunoglobulin domain (fold). Immunoglobulin superfamily ligands include, but are not limited to, CD80 and CD86, both ligands for CD28, or PD-L1/(B7-H1) that are ligands for PD-1. In certain embodiments, the at least one co-stimulatory ligand is selected from the group consisting of 4-1BBL, CD80, CD86, CD70, OX40L, CD48, TNFRSF14, PD-L1, and combinations thereof. In certain embodiments, the engineered immune cell comprises one recombinant co-stimulatory ligand that is 4-1BBL. In certain embodiments, the engineered immune cell comprises two recombinant co-stimulatory ligands that are 4-1BBL and CD80. CARs comprising at least one co-stimulatory ligand are described in U.S. Pat. No. 8,389,282, which is incorporated by reference in its entirety.

Furthermore, a presently disclosed engineered immune cells can further comprise at least one exogenous cytokine. For example, a presently disclosed engineered immune cell can be further transduced with at least one cytokine, such that the engineered immune cells secretes the at least one cytokine as well as expresses the tumor antigen-targeted CAR. In certain embodiments, the at least one cytokine is selected from the group consisting of IL-2, IL-3, IL-6, IL-7, IL-11, IL-12, IL-15, IL-17, and IL-21. In certain embodiments, the cytokine is IL-12.

The engineered immune cells can be generated from peripheral donor lymphocytes. The engineered immune cells (e.g., T cells) can be autologous, non-autologous (e.g., allogeneic), or derived in vitro from engineered progenitor or stem cells.

In certain embodiments, a presently disclosed engineered immune cells (e.g., T cells) expresses from about 1 to about 5, from about 1 to about 4, from about 2 to about 5, from about 2 to about 4, from about 3 to about 5, from about 3 to about 4, from about 4 to about 5, from about 1 to about 2, from about 2 to about 3, from about 3 to about 4, or from about 4 to about 5 vector copy numbers per cell of a presently disclosed tumor antigen-targeted CAR and/or anti-DOTA C825 antigen binding fragment.

For example, the higher the CAR expression level in an engineered immune cell, the greater cytotoxicity and cytokine production the engineered immune cell exhibits. An engineered immune cell (e.g., T cell) having a high tumor antigen-targeted CAR expression level can induce antigen-specific cytokine production or secretion and/or exhibit cytotoxicity to a tissue or a cell having a low expression level of tumor antigen-targeted CAR, e.g., about 2,000 or less, about 1,000 or less, about 900 or less, about 800 or less, about 700 or less, about 600 or less, about 500 or less, about 400 or less, about 300 or less, about 200 or less, about 100 or less of tumor antigen binding sites/cell. Additionally or alternatively, the cytotoxicity and cytokine production of a presently disclosed engineered immune cell (e.g., T cell) are proportional to the expression level of tumor antigen in a target tissue or a target cell. For example, the higher the expression level of human tumor antigen in the target, the greater cytotoxicity and cytokine production the engineered immune cell exhibits.

The unpurified source of immune cells may be any source known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-immune cell initially. Monoclonal antibodies are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. Suitably, at least about 80%, usually at least 70% of the total hematopoietic cells will be removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g., plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). Usually, the cells are collected in a medium comprising 2% fetal calf serum (FCS) or 0.2% bovine serum albumin (BSA) or any other suitable (e.g., sterile), isotonic medium.

In some embodiments, the engineered immune cells comprise one or more additional modifications. For example, in some embodiments, the engineered immune cells comprise and express (is transduced to express) an antigen recognizing receptor that binds to a second antigen that is different than selected tumor antigen. The inclusion of an antigen recognizing receptor in addition to a presently disclosed CAR on the engineered immune cell can increase the avidity of the CAR or the engineered immune cell comprising thereof on a targeted cell, especially, the CAR is one that has a low binding affinity to a particular tumor antigen, e.g., a K_{d} of about 2×10⁻⁸ M or more, about 5×10⁻⁸ M or more, about 8×10⁻⁸ M or more, about 9×10⁻⁸ M or more, about 1×10⁻⁷ M or more, about 2×10⁻⁷ M or more, or about 5×10⁻⁷ M or more.

In certain embodiments, the antigen recognizing receptor is a chimeric co-stimulatory receptor (CCR). CCR is described in Krause, et al., J. Exp. Med. 188(4):619-626(1998), and US20020018783, the contents of which are incorporated by reference in their entireties. CCRs mimic co-stimulatory signals, but unlike, CARs, do not provide a T-cell activation signal, e.g., CCRs lack a CD3ζ polypeptide. CCRs provide co-stimulation, e.g., a CD28-like signal, in the absence of the natural co-stimulatory ligand on the antigen-presenting cell. A combinatorial antigen recognition, i.e., use of a CCR in combination with a CAR, can augment T-cell reactivity against the dual-antigen expressing T cells, thereby improving selective tumor targeting. Kloss et al., describe a strategy that integrates combinatorial antigen recognition, split signaling, and, critically, balanced strength of T-cell activation and costimulation to generate T cells that eliminate target cells that express a combination of antigens while sparing cells that express each antigen individually (Kloss et al., Nature Biotechnology 31(1):71-75 (2013)). With this approach, T-cell activation requires CAR-mediated recognition of one antigen, whereas costimulation is independently mediated by a CCR specific for a second antigen. To achieve tumor selectivity, the combinatorial antigen recognition approach diminishes the efficiency of T-cell activation to a level where it is ineffective without rescue provided by simultaneous CCR recognition of the second antigen. In certain embodiments, the CCR comprises an extracellular antigen-binding domain that binds to an antigen different than selected tumor antigen, a transmembrane domain, and a co-stimulatory signaling region that comprises at least one co-stimulatory molecule, including, but not limited to, CD28, 4-1BB, OX40, ICOS, PD-1, CTLA-4, LAG-3, 2B4, and BTLA. In certain embodiments, the co-stimulatory signaling region of the CCR comprises one co-stimulatory signaling molecule. In certain embodiments, the one co-stimulatory signaling molecule is CD28. In certain embodiments, the one co-stimulatory signaling molecule is 4-1BB. In certain embodiments, the co-stimulatory signaling region of the CCR comprises two co-stimulatory signaling molecules. In certain embodiments, the two co-stimulatory signaling molecules are CD28 and 4-1BB. A second antigen is selected so that expression of both selected tumor antigen and the second antigen is restricted to the targeted cells (e.g., cancerous tissue or cancerous cells). Similar to a CAR, the extracellular antigen-binding domain can be a scFv, a Fab, a F(ab)₂; or a fusion protein with a heterologous sequence to form the extracellular antigen-binding domain. In certain embodiments, the CCR comprises a scFv that binds to CD138, transmembrane domain comprising a CD28 polypeptide, and a co-stimulatory signaling region comprising two co-stimulatory signaling molecules that are CD28 and 4-1BB.

In certain embodiments, the antigen recognizing receptor is a truncated CAR. A “truncated CAR” is different from a CAR by lacking an intracellular signaling domain. For example, a truncated CAR comprises an extracellular antigen-binding domain and a transmembrane domain, and lacks an intracellular signaling domain. In accordance with the presently disclosed subject matter, the truncated CAR has a high binding affinity to the second antigen expressed on the targeted cells, e.g., myeloma cells. The truncated CAR functions as an adhesion molecule that enhances the avidity of a presently disclosed CAR, especially, one that has a low binding affinity to tumor antigen, thereby improving the efficacy of the presently disclosed CAR or engineered immune cell (e.g., T cell) comprising thereof. In certain embodiments, the truncated CAR comprises an extracellular antigen-binding domain that binds to CD138, a transmembrane domain comprising a CD8 polypeptide. A presently disclosed T cell comprises or is transduced to express a presently disclosed CAR targeting tumor antigen and a truncated CAR targeting CD138. In certain embodiments, the targeted cells are solid tumor cells. In some embodiments, the engineered immune cells are further modified to suppress expression of one or more genes. In some embodiments, the engineered immune cells are further modified via genome editing. Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus. See, for example, U.S. Pat. Nos. 7,888,121; 7,972,854; 7,914,796; 7,951,925; 8,110,379; 8,409,861; 8,586,526; U.S. Patent Publications 20030232410; 20050208489; 20050026157; 20050064474; 20060063231; 201000218264; 20120017290; 20110265198; 20130137104; 20130122591; 20130177983 and 20130177960, the disclosures of which are incorporated by reference in their entireties. These methods often involve the use of engineered cleavage systems to induce a double strand break (DSB) or a nick in a target DNA sequence such that repair of the break by an error born process such as non-homologous end joining (NHEJ) or repair using a repair template (homology directed repair or HDR) can result in the knock out of a gene or the insertion of a sequence of interest (targeted integration). Cleavage can occur through the use of specific nucleases such as engineered zinc finger nucleases (ZFN), transcription-activator like effector nucleases (TALENs), or using the CRISPR/Cas system with an engineered crRNA/tracr RNA (‘single guide RNA’) to guide specific cleavage. In some embodiments, the engineered immune cells are modified to disrupt or reduce expression of an endogenous T-cell receptor gene (see, e.g., WO 2014153470, which is incorporated by reference in its entirety). In some embodiments, the engineered immune cells are modified to result in disruption or inhibition of PD1, PDL-1 or CTLA-4 (see, e.g., U.S. Patent Publication 20140120622), or other immunosuppressive factors known in the art (Wu et al. (2015) Oncoimmunology 4(7): e1016700, Mahoney et al. (2015) Nature Reviews Drug Discovery 14, 561-584).

### Vectors

Many expression vectors are available and known to those of skill in the art and can be used for expression of polypeptides provided herein. The choice of expression vector will be influenced by the choice of host expression system. Such selection is well within the level of skill of the skilled artisan. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector in the cells.

Vectors also can contain additional nucleotide sequences operably linked to the ligated nucleic acid molecule, such as, for example, an epitope tag such as for localization, e.g., a hexa-his tag (SEQ ID NO: 43) or a myc tag, hemagglutinin tag or a tag for purification, for example, a GST fusion, and a sequence for directing protein secretion and/or membrane association.

Expression of the antibodies or antigen-binding fragments thereof can be controlled by any promoter/enhancer known in the art. Suitable bacterial promoters are well known in the art and described herein below. Other suitable promoters for mammalian cells, yeast cells and insect cells are well known in the art and some are exemplified below. Selection of the promoter used to direct expression of a heterologous nucleic acid depends on the particular application and is within the level of skill of the skilled artisan. Promoters which can be used include but are not limited to eukaryotic expression vectors containing the SV40 early promoter (Bernoist and Chambon, Nature 290:304-310(1981)), the promoter contained in the 3′ long terminal repeat of Rous sarcoma virus (Yamamoto et al., Cell 22:787-797(1980)), the herpes thymidine kinase promoter (Wagner et al., Proc. Natl. Acad. Sci. USA 75: 1441-1445 (1981)), the regulatory sequences of the metallothionein gene (Brinster et al., Nature 296:39-42 (1982)); prokaryotic expression vectors such as the (3-lactamase promoter (Jay et al., Proc. Natl. Acad. Sci. USA 75:5543 (1981)) or the tac promoter (DeBoer et al., Proc. Natl. Acad. Sci. USA 50:21-25(1983)); see also “Useful Proteins from Recombinant Bacteria”: in Scientific American 242:79-94 (1980)); plant expression vectors containing the nopaline synthetase promoter (Herrera-Estrella et al., Nature 505:209-213(1984)) or the cauliflower mosaic virus 35S RNA promoter (Gardner et al., Nucleic Acids Res. 9:2871(1981)), and the promoter of the photosynthetic enzyme ribulose bisphosphate carboxylase (Herrera-Estrella et al., Nature 510: 1 15-120(1984)); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 55:639-646 (1984); Ornitz et al., Cold Spring Harbor Symp. Quant. Biol. 50:399-409(1986); MacDonald, Hepatology 7:425-515 (1987)); insulin gene control region which is active in pancreatic beta cells (Hanahan et al., Nature 515: 115-122 (1985)), immunoglobulin gene control region which is active in lymphoid cells (Grosschedl et al., Cell 55:647-658 (1984); Adams et al., Nature 515:533-538 (1985); Alexander et al., Mol. Cell Biol. 7: 1436-1444 (1987)), mouse mammary tumor virus control region which is active in testicular, breast, lymphoid and mast cells (Leder et al., Cell 15:485-495 (1986)), albumin gene control region which is active in liver (Pinckert et al., Genes and Devel. 1:268-276 (1987)), alpha-fetoprotein gene control region which is active in liver (Krumlauf et al., Mol. Cell. Biol. 5:1639-403 (1985)); Hammer et al., Science 255:53-58 (1987)), alpha-1 antitrypsin gene control region which is active in liver (Kelsey et al., Genes and Devel. 7:161-171 (1987)), beta globin gene control region which is active in myeloid cells (Magram et al., Nature 515:338-340 (1985)); Kollias et al., Cell 5:89-94 (1986)), myelin basic protein gene control region which is active in oligodendrocyte cells of the brain (Readhead et al., Cell 15:703-712 (1987)), myosin light chain-2 gene control region which is active in skeletal muscle (Shani, Nature 514:283-286 (1985)), and gonadotrophic releasing hormone gene control region which is active in gonadotrophs of the hypothalamus (Mason et al., Science 254: 1372-1378 (1986)).

In addition to the promoter, the expression vector typically contains a transcription unit or expression cassette that contains all the additional elements required for the expression of the antibody, or portion thereof, in host cells. A typical expression cassette contains a promoter operably linked to the nucleic acid sequence encoding the antibody chain and signals required for efficient polyadenylation of the transcript, ribosome binding sites and translation termination. Additional elements of the cassette can include enhancers. In addition, the cassette typically contains a transcription termination region downstream of the structural gene to provide for efficient termination. The termination region can be obtained from the same gene as the promoter sequence or can be obtained from different genes.

Some expression systems have markers that provide gene amplification such as thymidine kinase and dihydrofolate reductase. Alternatively, high yield expression systems not involving gene amplification are also suitable, such as using a baculovirus vector in insect cells, with a nucleic acid sequence encoding a germline antibody chain under the direction of the polyhedron promoter or other strong baculovirus promoter.

Any methods known to those of skill in the art for the insertion of DNA fragments into a vector can be used to construct expression vectors containing a nucleic acid encoding any of the polypeptides provided herein. These methods can include in vitro recombinant DNA and synthetic techniques and in vivo recombinants (genetic recombination). The insertion into a cloning vector can, for example, be accomplished by ligating the DNA fragment into a cloning vector which has complementary cohesive termini. If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized nucleic acids encoding restriction endonuclease recognition sequences.

Exemplary plasmid vectors useful to produce the polypeptides provided herein contain a strong promoter, such as the HCMV immediate early enhancer/promoter or the MHC class I promoter, an intron to enhance processing of the transcript, such as the HCMV immediate early gene intron A, and a polyadenylation (poly A) signal, such as the late SV40 polyA signal.

Genetic modification of engineered immune cells (e.g., T cells, NK cells) can be accomplished by transducing a substantially homogeneous cell composition with a recombinant DNA or RNA construct. The vector can be a retroviral vector (e.g., gamma retroviral), which is employed for the introduction of the DNA or RNA construct into the host cell genome. For example, a polynucleotide encoding the tumor antigen-targeted CAR and the anti-DOTA C825 antigen binding fragment can be cloned into a retroviral vector and expression can be driven from its endogenous promoter, from the retroviral long terminal repeat, or from an alternative internal promoter.

Non-viral vectors or RNA may be used as well. Random chromosomal integration, or targeted integration (e.g., using a nuclease, transcription activator-like effector nucleases (TALENs), Zinc-finger nucleases (ZFNs), and/or clustered regularly interspaced short palindromic repeats (CRISPRs), or transgene expression (e.g., using a natural or chemically modified RNA) can be used.

For initial genetic modification of the cells to provide tumor antigen-targeted CAR and the anti-DOTA C825 antigen binding fragment expressing cells, a retroviral vector is generally employed for transduction, however any other suitable viral vector or non-viral delivery system can be used. For subsequent genetic modification of the cells to provide cells comprising an antigen presenting complex comprising at least two co-stimulatory ligands, retroviral gene transfer (transduction) likewise proves effective. Combinations of retroviral vector and an appropriate packaging line are also suitable, where the capsid proteins will be functional for infecting human cells. Various amphotropic virus-producing cell lines are known, including, but not limited to, PA12 (Miller, et al., Mol. Cell. Biol. 5:431-437 (1985)); PA317 (Miller, et al., Mol. Cell. Biol. 6:2895-2902 (1986)); and CRIP (Danos, et al. Proc. Natl. Acad. Sci. USA 85:6460-6464 (1988)). Non-amphotropic particles are suitable too, e.g., particles pseudotyped with VSVG, RD 114 or GALV envelope and any other known in the art.

Possible methods of transduction also include direct co-culture of the cells with producer cells, e.g., by the method of Bregni, et al., Blood 80: 1418-1422(1992), or culturing with viral supernatant alone or concentrated vector stocks with or without appropriate growth factors and polycations, e.g., by the method of Xu, et al., Exp. Hemat. 22:223-230 (1994); and Hughes, et al., J. Clin. Invest. 89: 1817 (1992).

Transducing viral vectors can be used to express a co-stimulatory ligand and/or secretes a cytokine (e.g., 4-1BBL and/or IL-12) in an engineered immune cell. In some embodiments, the chosen vector exhibits high efficiency of infection and stable integration and expression (see, e.g., Cayouette et al., Human Gene Therapy 8:423-430 (1997); Kido et al., Current Eye Research 15:833-844 (1996); Bloomer et al., Journal of Virology 71:6641-6649, 1997; Naldini et al., Science 272:263 267 (1996); and Miyoshi et al., Proc. Natl. Acad. Sci. U.S.A. 94: 10319, (1997)). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, (1990); Friedman, Science 244: 1275-1281 (1989); Eglitis et al., BioTechniques 6:608-614, (1988); Tolstoshev et al., Current Opinion in Biotechnology 1:55-61(1990); Sharp, The Lancet 337: 1277-1278 (1991); Cornetta et al., Nucleic Acid Research and Molecular Biology 36:311-322 (1987); Anderson, Science 226:401-409 (1984); Moen, Blood Cells 17:407-416 (1991); Miller et al., Biotechnology 7:980-990 (1989); Le Gal La Salle et al., Science 259:988-990 (1993); and Johnson, Chest 107:77S-83S (1995)). Retroviral vectors are particularly well developed and have been used in clinical settings (Rosenberg et al., N. Engl. J. Med 323:370 (1990); Anderson et al., U.S. Pat. No. 5,399,346).

In certain non-limiting embodiments, the vector expressing a presently disclosed tumor antigen-targeted CAR is a retroviral vector, e.g., an oncoretroviral vector.

Non-viral approaches can also be employed for the expression of a protein in cell. For example, a nucleic acid molecule can be introduced into a cell by administering the nucleic acid in the presence of lipofection (Feigner et al., Proc. Nat'l. Acad. Sci. U.S.A. 84:7413, (1987); Ono et al., Neuroscience Letters 17:259 (1990); Brigham et al., Am. J Med. Sci. 298:278, (1989); Staubinger et al., Methods in Enzymology 101:512 (1983)), asialoorosomucoid-polylysine conjugation (Wu et al., Journal of Biological Chemistry 263 14621 (1988); Wu et al., Journal of Biological Chemistry 264: 16985 (1989)), or by micro-injection under surgical conditions (Wolff et al., Science 247: 1465 (1990)). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g., Zinc finger nucleases, meganucleases, or TALE nucleases). Transient expression may be obtained by RNA electroporation.

cDNA expression can be directed from any suitable promoter (e.g., the human cytomegalovirus (CMV), simian virus 40 (SV40), or metallothionein promoters), and regulated by any appropriate mammalian regulatory element or intron (e.g., the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

### Polypeptides and Analogs and Polynucleotides

Also included in the presently disclosed subject matter are extracellular antigen-binding domains that specifically binds to a tumor antigen (e.g., human tumor antigen) (e.g., an scFv (e.g., a human scFv), a Fab, or a (Fab)₂), CD3ζ, CD8, CD28, etc. polypeptides or fragments thereof, and polynucleotides encoding thereof that are expressed in an engineered immune cell. The presently disclosed subject matter provides methods for optimizing an amino acid sequence or a nucleic acid sequence by producing an alteration in the sequence. Such alterations may comprise certain mutations, deletions, insertions, or post-translational modifications. The presently disclosed subject matter further comprises analogs of any naturally-occurring polypeptide of the presently disclosed subject matter. Analogs can differ from a naturally-occurring polypeptide of the presently disclosed subject matter by amino acid sequence differences, by post-translational modifications, or by both. Analogs of the presently disclosed subject matter can generally exhibit at least about 85%, about 90%, about 91%, about 92%, about 93%, about 94%, about 95%, about 96%, about 97%, about 98%, about 99% or more identity or homology with all or part of a naturally-occurring amino acid sequence of the presently disclosed subject matter. The length of sequence comparison is at least about 5, about 10, about 15, about 20, about 25, about 50, about 75, about 100 or more amino acid residues. Again, in an exemplary approach to determining the degree of identity, a BLAST program may be used, with a probability score between e⁻³ and e⁻¹⁰⁰ indicating a closely related sequence. Modifications comprise in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the presently disclosed subject matter by alterations in primary sequence. These include genetic variants, both natural and induced (for example, resulting from random mutagenesis by irradiation or exposure to ethanemethyl sulfate or by site-specific mutagenesis as described in Sambrook, Fritsch and Maniatis, Molecular Cloning: A Laboratory Manual (2nd ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., beta (β) or gamma (γ) amino acids.

In addition to full-length polypeptides, the presently disclosed subject matter also provides fragments of any one of the polypeptides or peptide domains of the presently disclosed subject matter. A fragment can be at least about 5, about 10, about 13, or about 15 amino acids. In some embodiments, a fragment is at least about 20 contiguous amino acids, at least about 30 contiguous amino acids, or at least about 50 contiguous amino acids. In some embodiments, a fragment is at least about 60 to about 80, about 100, about 200, about 300 or more contiguous amino acids. Fragments of the presently disclosed subject matter can be generated by methods known to those of ordinary skill in the art or may result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein of the present technology. Such analogs are administered according to methods of the presently disclosed subject matter. Such analogs may exceed the physiological activity of the original polypeptide. Methods of analog design are well known in the art, and synthesis of analogs can be carried out according to such methods by modifying the chemical structures such that the resultant analogs enhance the function of the original polypeptide when expressed in an engineered immune cell. These chemical modifications include, but are not limited to, substituting alternative R groups and varying the degree of saturation at specific carbon atoms of a reference polypeptide. The protein analogs can be relatively resistant to in vivo degradation, resulting in a more prolonged effect upon administration. Assays for measuring functional activity include, but are not limited to, those described in the Examples below.

In accordance with the presently disclosed subject matter, the polynucleotides encoding an extracellular antigen-binding domain that specifically binds to tumor antigen (e.g., human tumor antigen) (e.g., an scFv (e.g., a human scFv), a Fab, or a (Fab)₂), CD3, CD8, CD28 can be modified by codon optimization. Codon optimization can alter both naturally occurring and recombinant gene sequences to achieve the highest possible levels of productivity in any given expression system. Factors that are involved in different stages of protein expression include codon adaptability, mRNA structure, and various cis-elements in transcription and translation. Any suitable codon optimization methods or technologies that are known to ones skilled in the art can be used to modify the polynucleotides of the presently disclosed subject matter, including, but not limited to, OptimumGene™, Encor optimization, and Blue Heron.

### Administration

Engineered immune cells expressing the tumor antigen-targeted CAR and an anti-DOTA C825 antigen binding fragment of the presently disclosed subject matter can be provided systemically or directly to a subject for diagnosing or monitoring progression of a neoplasia. In certain embodiments, engineered immune cells are directly injected into an organ of interest (e.g., an organ affected by a neoplasia). Alternatively or additionally, the engineered immune cells are provided indirectly to the organ of interest, for example, by administration into the circulatory system (e.g., the tumor vasculature) or into the solid tumor. Expansion and differentiation agents can be provided prior to, during or after administration of cells and compositions to increase production of T cells in vitro or in vivo.

Engineered immune cells of the presently disclosed subject matter can be administered in any physiologically acceptable vehicle, systemically or regionally, normally intravascularly, intraperitoneally, intrathecally, or intrapleurally, although they may also be introduced into bone or other convenient site where the cells may find an appropriate site for regeneration and differentiation (e.g., thymus). In certain embodiments, at least 1×10⁵ cells can be administered, eventually reaching 1×10¹⁰ or more. In certain embodiments, at least 1×10⁶ cells can be administered. A cell population comprising engineered immune cells can comprise a purified population of cells. Those skilled in the art can readily determine the percentage of engineered immune cells in a cell population using various well-known methods, such as fluorescence activated cell sorting (FACS). The ranges of purity in cell populations comprising engineered immune cells can be from about 50% to about 55%, from about 55% to about 60%, from about 65% to about 70%, from about 70% to about 75%, from about 75% to about 80%, from about 80% to about 85%; from about 85% to about 90%, from about 90% to about 95%, or from about 95 to about 100%. Dosages can be readily adjusted by those skilled in the art (e.g., a decrease in purity may require an increase in dosage). The engineered immune cells can be introduced by injection, catheter, or the like. If desired, factors can also be included, including, but not limited to, interleukins, e.g., IL-2, IL-3, IL 6, IL-11, IL-7, IL-12, IL-15, IL-21, as well as the other interleukins, the colony stimulating factors, such as G-, M- and GM-CSF, interferons, e.g., γ-interferon.

In certain embodiments, compositions of the presently disclosed subject matter comprise pharmaceutical compositions comprising engineered immune cells expressing a tumor antigen-targeted CAR and an anti-DOTA C825 antigen binding fragment with a pharmaceutically acceptable carrier. Administration can be autologous or non-autologous. For example, engineered immune cells expressing a tumor antigen-targeted CAR and an anti-DOTA C825 antigen binding fragment and compositions comprising thereof can be obtained from one subject, and administered to the same subject or a different, compatible subject. Peripheral blood derived T cells of the presently disclosed subject matter or their progeny (e.g., in vivo, ex vivo or in vitro derived) can be administered via localized injection, including catheter administration, systemic injection, localized injection, intravenous injection, or parenteral administration. When administering a pharmaceutical composition of the presently disclosed subject matter (e.g., a pharmaceutical composition comprising engineered immune cells expressing a tumor antigen-targeted CAR), it can be formulated in a unit dosage injectable form (solution, suspension, emulsion).

### Formulations

Engineered immune cells expressing a tumor antigen-targeted CAR and an anti-DOTA C825 antigen binding fragment and compositions comprising thereof can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the compositions of the presently disclosed subject matter, e.g., a composition comprising engineered immune cells, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the engineered immune cells of the presently disclosed subject matter.

The compositions can be isotonic, i.e., they can have the same osmotic pressure as blood and lacrimal fluid. The desired isotonicity of the compositions of the presently disclosed subject matter may be accomplished using sodium chloride, or other pharmaceutically acceptable agents such as dextrose, boric acid, sodium tartrate, propylene glycol or other inorganic or organic solutes. Sodium chloride is suitable particularly for buffers containing sodium ions.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Those skilled in the art will recognize that the components of the compositions should be selected to be chemically inert and will not affect the viability or efficacy of the engineered immune cells as described in the presently disclosed subject matter. This will present no problem to those skilled in chemical and pharmaceutical principles, or problems can be readily avoided by reference to standard texts or by simple experiments (not involving undue experimentation), from this disclosure and the documents cited herein.

One consideration concerning the use of the engineered immune cells of the presently disclosed subject matter is the quantity of cells necessary to achieve an optimal effect. The quantity of cells to be administered will vary according to the subject. In certain embodiments, from about 10² to about 10¹², from about 10³ to about 10¹¹, from about 10⁴ to about 10¹⁰, from about 10⁵ to about 10⁹, or from about 10⁶ to about 10⁸ engineered immune cells of the presently disclosed subject matter are administered to a subject. More effective cells may be administered in even smaller numbers. In some embodiments, at least about 1×10⁸, about 2×10⁸, about 3×10⁸, about 4×10⁸, about 5×10⁸, about 1×10⁹, about 5×10⁹, about 1×10¹⁰, about 5×10¹⁰, about 1×10¹¹, about 5×10¹¹, about 1×10¹² or more engineered immune cells of the presently disclosed subject matter are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art. Generally, engineered immune cells are administered at doses that are nontoxic or tolerable to the patient.

The skilled artisan can readily determine the amount of cells and optional additives, vehicles, and/or carrier in compositions to be administered in methods of the presently disclosed subject matter. Typically, any additives (in addition to the active cell(s) and/or agent(s)) are present in an amount of from about 0.001% to about 50% by weight) solution in phosphate buffered saline, and the active ingredient is present in the order of micrograms to milligrams, such as from about 0.0001 wt % to about 5 wt %, from about 0.0001 wt % to about 1 wt %, from about 0.0001 wt % to about 0.05 wt %, from about 0.001 wt % to about 20 wt %, from about 0.01 wt % to about 10 wt %, or from about 0.05 wt % to about 5 wt %. For any composition to be administered to an animal or human, and for any particular method of administration, toxicity should be determined, such as by determining the lethal dose (LD) and LD50 in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### DOTA Hapten Compositions

DOTA is a macrocyclic chelating agent that forms stable metal complexes that are irreversible under physiological conditions. DOTA has a molecular weight of 405 Daltons, and exhibits rapid diffusion and renal clearance.

Examples of DOTA haptens include, but are not limited to, benzyl-DOTA, NH₂-benzyl (Bn) DOTA, DOTA-desferrioxamine, DOTA-Phe-Lys(HSG)-D-Tyr-Lys(HSG)-NH₂, Ac-Lys(HSG)D-Tyr-Lys(HSG)-Lys(Tscg-Cys)-NH₂, DOTA-D-Asp-D-Lys(HSG)-D-Asp-D-Lys(HSG)-NH₂; DOTA-D-Glu-D-Lys(HSG)-D-Glu-D-Lys(HSG)-NH₂, DOTA-D-Tyr-D-Lys(HSG)-D-Glu-D-Lys(HSG)-NH₂, DOTA-D-Ala-D-Lys(HSG)-D-Glu-D-Lys(HSG)-NH₂, DOTA-D-Phe-D-Lys(HSG)-D-Tyr-D-Lys(HSG)-NH₂, Ac-D-Phe-D-Lys(DOTA)-D-Tyr-D-Lys(DOTA)-NH₂, Ac-D-Phe-D-Lys(DTPA)-D-Tyr-D-Lys(DTPA)-NH₂, Ac-D-Phe-D-Lys(Bz-DTPA)-D-Tyr-D-Lys(Bz-DTPA)-NH₂, Ac-D-Lys(HSG)-D-Tyr-D-Lys(HSG)-D-Lys(Tscg-Cys)-NH₂, DOTA-D-Phe-D-Lys(HSG)-D-Tyr-D-Lys(HSG)-D-Lys(Tscg-Cys)-NH₂, (Tscg-Cys)-D-Phe-D-Lys(HSG)-D-Tyr-D-Lys(HSG)-D-Lys(DOTA)-NH₂, Tscg-D-Cys-D-Glu-D-Lys(HSG)-D-Glu-D-Lys(HSG)-NH₂, (Tscg-Cys)-D-Glu-D-Lys(HSG)-D-Glu-D-Lys(HSG)-NH₂, Ac-D-Cys-D-Lys(DOTA)-D-Tyr-D-Ala-D-Lys(DOTA)-D-Cys-NH₂, Ac-D-Cys-D-Lys(DTPA)-D-Tyr-D-Lys(DTPA)-NH₂, Ac-D-Lys(DTPA)-D-Tyr-D-Lys(DTPA)-D-Lys(Tscg-Cys)-NH₂, Ac-D-Lys(DOTA)-D-Tyr-D-Lys(DOTA)-D-Lys(Tscg-Cys)-NH₂, DOTA-RGD, DOTA-PEG-E(c(RGDyK))₂, DOTA-8-AOC-BBN, DOTA-PESIN, p-NO2-benzyl-DOTA, DOTA-biotin-sarcosine (DOTA-biotin), 1,4,7,10-tetraazacyclododecane-1,4,7,10-tetraacetic acid mono (N-hydroxysuccinimide ester) (DOTA-NHS), and DOTATyrLysDOTA. See Orcutt et al., Mol Imaging Biol. (2011) April; 13(2): 215-221; Cheal S M, et al. (2017) J Nucl Med, 58(11):1735-42 (describing DOTA haptens with β-emitters); Cheal S M, et al. (2018) J Nucl Med, 59:123 (α-emitters). DOTA and its variants chelate a wide range of metals including paramagnetic metals and radionuclides. Exemplary metals include indium, gallium, gadolinium, europium, terbium, copper, bismuth, and the like.

In some embodiments, the DOTA hapten has the structure of Formula I

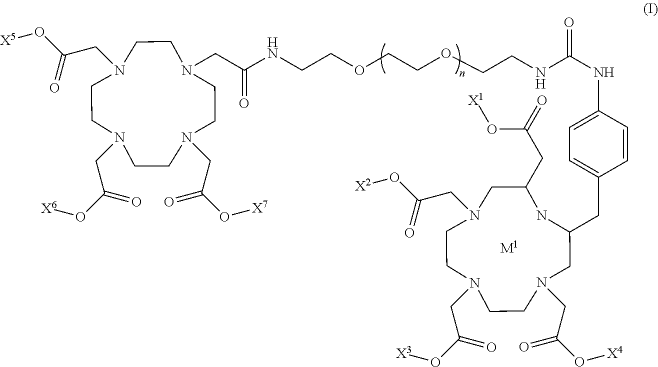

or a pharmaceutically acceptable salt thereof, wherein M¹ is ¹⁷⁵Lu³⁺, ⁴⁵Sc³⁺, ⁶⁹Ga³⁺, ⁷¹Ga³⁺, ⁸⁹Y³⁺, ¹¹³In³⁺, ¹¹⁵In³⁺, ¹³⁹La³⁺, ¹³⁶Ce³⁺, 138Ce³⁺, ¹⁴⁰Ce³⁺, ¹⁴²Ce³⁺, ¹⁵¹Eu³⁺, ¹⁵³Eu³⁺, ¹⁵⁹Tb³⁺, ¹⁵⁴Gd³⁺, ¹⁵⁵Gd³⁺, ¹⁵⁶Gd³⁺, ¹⁵⁷Gd³⁺, ¹⁵⁸Gd³⁺, or ¹⁶⁰Gd³⁺; X¹, X², X³, and X⁴ are each independently a lone pair of electrons (i.e., providing an oxygen anion) or H; X⁵, X⁶, and X⁷ are each independently a lone pair of electrons (i.e., providing an oxygen anion) or H; and n is 1, 2, 3, 4, 5, 6, 7, 8, 9, 10, 11, 12, 13, 14, 15, 16, 17, 18, 19, 20, 21, or 22. In certain embodiments, n is 3.

In some embodiments of the DOTA hapten, at least two of X¹, X², X³, and X⁴ are each independently a lone pair of electrons. In certain embodiments of the DOTA hapten, three of X¹, X², X³, and X⁴ are each independently a lone pair of electrons and the remaining X¹, X², X³, or X⁴ is H.

Additionally or alternatively, in some embodiments, the present disclosure provides a bischelate comprising any of the above DOTA haptens of Formula I and a radionuclide cation. In some embodiments, the DOTA hapten of Formula I can bind a radionuclide cation with a K_{d} of about 1 pM-1 nM (e.g., about 1-10 pM; 1-100 pM; 5-50 pM; 100-500 pM; or 500 pM-1 nM). In some embodiments, the K_{d} is in the range of about 1 nM to about 1 pM, for example, no more than about 1 nM, 950 pM, 900 pM, 850 pM, 800 pM, 750 pM, 700 pM, 650 pM, 600 pM, 550 pM, 500 pM, 450 pM, 400 pM, 350 pM, 300 pM, 250 pM, 200 pM, 150 pM, 100 pM, 90 pM, 80 pM, 70 pM, 60 pM, 50 pM, 40 pM, 30 pM, 20 pM, 10 pM, 9 pM, 8 pM, 7 pM, 6 pM, 5 pM, 4 pM, 3 pM, 2.5 pM, 2 pM, or 1 pM. In some embodiments, the bischelate is of Formula II

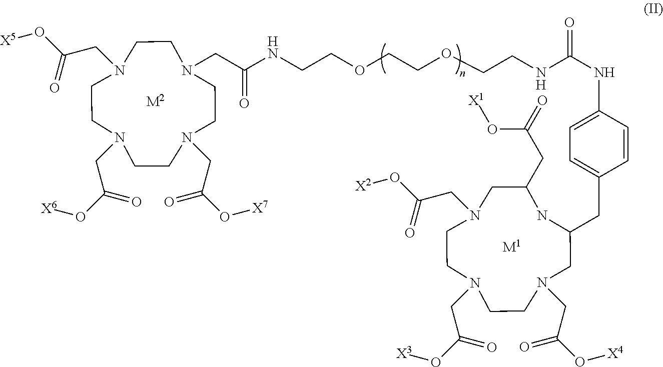

or a pharmaceutically acceptable salt thereof, wherein M¹ is ¹⁷⁵Lu³⁺, ⁴⁵Sc³⁺, ⁶⁹Ga³⁺, ⁷¹Ga³⁺, ⁸⁹Y³⁺, ¹¹³In³⁺, ¹¹⁵In³⁺, ¹³⁹La³⁺, ¹³⁶Ce³⁺, 138Ce³⁺, ¹⁴⁰Ce³⁺, ¹⁴²Ce³⁺, ¹⁵¹Eu³⁺, ¹⁵³Eu³⁺, ¹⁵⁹Tb³⁺, ¹⁵⁴Gd³⁺, ¹⁵⁵Gd³⁺, ¹⁵⁶Gd³⁺, ¹⁵⁷Gd³⁺, ¹⁵⁸Gd³⁺, or ¹⁶⁰Gd³⁺; M² is the radionuclide cation; X¹, X², X³, and X⁴ are each independently a lone pair of electrons (i.e., providing an oxygen anion) or H; X⁵, X⁶, and X⁷ are each independently a lone pair of electrons (i.e., providing an oxygen anion) or H; and n is 1, 2, 3, 4, 5, 6, 7, 8, 9, 10, 11, 12, 13, 14, 15, 16, 17, 18, 19, 20, 21, or 22. In certain embodiments, n is 3. In some embodiments of the bischelate, at least two of X⁵, X⁶, and X⁷ are each independently a lone pair of electrons. Additionally or alternatively, in some embodiments of the bischelate, the radionuclide cation is a divalent cation or a trivalent cation.

In any and all embodiments of the DOTA haptens disclosed herein, M² is ¹¹¹In, ⁶⁷Ga, ⁵¹Cr, ⁵⁸Co, ^{99m}Tc, ^{103m}Rh, ^{195m}Pt, ¹¹⁹Sb, ¹⁶¹Ho, ^{189m}Os, ¹⁹²Ir, ²⁰¹Tl, ²⁰³Pb, ⁸⁹Zr, ⁶⁸Ga, or ⁶⁴Cu.

In another aspect, the present disclosure provides a complex comprising an engineered immune cell provided herein and a DOTA hapten, wherein the engineered immune cell is configured to bind to the DOTA hapten and a tumor antigen. The present disclosure also provides a complex comprising a bischelate (e.g., the bischelate of Formula II) and an engineered immune cell, wherein the engineered immune cell is configured to bind to the DOTA hapten and a tumor antigen. In any of the above embodiments of the complexes disclosed herein, the engineered immune cell expresses an anti-DOTA C825 antigen binding fragment (See Cheal et al., Mol Cancer Ther. 13(7):1803-12 (2014)). Additionally or alternatively, in any of the above embodiments of the complexes disclosed herein, the engineered immune cell expresses an anti-DOTA C825 antigen binding fragment with a G54C substitution.

In any of the above embodiments of the complexes disclosed herein, the tumor antigen is selected from the group consisting of 5T4, alpha 5β1-integrin, 707-AP, A33, AFP, ART-4, B7H4, BAGE, Bcl-2, β-catenin, BCMA, Bcr-abl, MN/C IX antibody, CA125, CA19-9, CAMEL, CAP-1, CASP-8, CD4, CD5, CD19, CD20, CD21, CD22, CD25, CDC27/m, CD33, CD37, CD45, CD52, CD56, CD80, CD123, CDK4/m, CEA, c-Met, CS-1, CT, Cyp-B, cyclin B1, DAGE, DAM, EBNA, EGFR, ErbB3, ELF2M, EMMPRIN, EpCam, ephrinB2, estrogen receptor, ETV6-AML1, FAP, ferritin, folate-binding protein, GAGE, G250, GD-2, GM2, GnT-V, gp75, gp100 (Pmel 17), HAGE, HER-2/neu, HLA-A*0201-R170I, HPV E6, HPV E7, Ki-67, HSP70-2M, HST-2, hTERT (or hTRT), iCE, IGF-1R, IL-2R, IL-5, KIAA0205, LAGE, LDLR/FUT, LRP, MAGE, MART, MART-1/melan-A, MART-2/Ski, MC1R, mesothelin, MUC, MUC16, MUM-1-B, myc, MUM-2, MUM-3, NA88-A, NYESO-1, NY-Eso-B, p53, proteinase-3, p190 minor bcr-abl, Pml/RARα, PRAME, progesterone receptor, PSA, PSCA, PSM, PSMA, ras, RAGE, RU1 or RU2, RORI, SART-1 or SART-3, survivin, TEL/AML1, TGFβ, TPI/m, TRP-1, TRP-2, TRP-2/INT2, tenascin, TSTA tyrosinase, VEGF, and WT1. Additionally or alternatively, in some embodiments of the complex, the anti-DOTA C825 antigen binding fragment of the engineered immune cell binds to the DOTA hapten with a K_{d} that is lower than or equal to 100 nM-95 nM, 95-90 nM, 90-85 nM, 85-80 nM, 80-75 nM, 75-70 nM, 70-65 nM, 65-60 nM, 60-55 nM, 55-50 nM, 50-45 nM, 45-40 nM, 40-35 nM, 35-30 nM, 30-25 nM, 25-20 nM, 20-15 nM, 15-10 nM, 10⁻⁵ nM, 5-1 nM, 1 nM-950 pM, 950 pM-900 pM, 900 pM-850 pM, 850 pM-800 pM, 800 pM-750 pM, 750 pM-700 pM, 700 pM-650 pM, 650 pM-600 pM, 600 pM-550 pM, 550 pM-500 pM, 500 pM-450 pM, 450 pM-400 pM, 400 pM-350 pM, 350 pM-300 pM, 300 pM-250 pM, 250 pM-200 pM, 200 pM-150 pM, 150 pM-100 pM, 100 pM-50 pM, 50 pM-40 pM, 40 pM-30 pM, 30 pM-20 pM, 20 pM-10 pM, 9 pM, 8 pM, 7 pM, 6 pM, 5 pM, 4 pM, 3 pM, 2.5 pM, 2 pM, 1.5 pM, or 1 pM.

### Diagnostic Methods of the Present Technology

In one aspect, the present disclosure provides a method for detecting tumors (e.g., solid or liquid tumors) in a subject in need thereof comprising (a) administering to the subject an effective amount of any complex of the present technology, wherein the complex is configured to localize to a tumor expressing the tumor antigen recognized by the engineered immune cell of the complex; and (b) detecting the presence of tumors in the subject by detecting radioactive levels emitted by the complex that are higher than a reference value. Also disclosed are methods for detecting tumors (e.g., solid or liquid tumors) in a subject in need thereof comprising (a) administering to the subject an effective amount of any engineered immune cell described herein, wherein the engineered immune cell is configured to localize to a tumor expressing the tumor antigen recognized by the engineered immune cell; (b) administering to the subject an effective amount of a radiolabeled-DOTA hapten, wherein the radiolabeled-DOTA hapten is configured to bind to the anti-DOTA C825 antigen binding fragment expressed by the engineered immune cell; and (c) detecting the presence of tumors in the subject by detecting radioactive levels emitted by the radiolabeled-DOTA hapten that are higher than a reference value.

Additionally or alternatively, in some embodiments of the methods disclosed herein, the radioactive levels emitted by the complex or the radiolabeled-DOTA hapten are detected using positron emission tomography or single photon emission computed tomography.

In some embodiments of the methods disclosed herein, the subject is diagnosed with, or is suspected of having cancer. Examples of cancer include, but are not limited to, adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.

In any of the preceding embodiments of the methods disclosed herein, the complex, the engineered immune cell, or the radiolabeled-DOTA hapten is administered intravenously, intratumorally, intramuscularly, intraarterially, intrathecally, intracapsularly, intraorbitally, intradermally, intraperitoneally, transtracheally, subcutaneously, intracerebroventricularly, orally or intranasally. Additionally or alternatively, in some embodiments, the complex, the engineered immune cell, or the radiolabeled-DOTA hapten is administered into the cerebral spinal fluid or blood of the subject.

Additionally or alternatively, in some embodiments, the radioactive levels emitted by the complex or the radiolabeled-DOTA hapten are detected between 2 to 120 hours after the complex or the radiolabeled-DOTA hapten is administered. In certain embodiments, the radioactive levels emitted by the complex or the radiolabeled-DOTA hapten are expressed as the percentage injected dose per gram tissue (0% ID/g). The reference value may be calculated by measuring the radioactive levels present in non-tumor (normal) tissues, and computing the average radioactive levels present in non-tumor (normal) tissues±standard deviation. In some embodiments, the reference value is the standard uptake value (SUV). See Thie J A, J Nucl Med. 45(9):1431-4 (2004). Additionally or alternatively, in some embodiments, the ratio of radioactive levels between a tumor and normal tissue is about 2:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1 or 100:1. In some embodiments, the subject is human.

The radiolabeled-DOTA hapten may be administered at any time between 1 minute to 4 or more days following administration of the engineered immune cells expressing the anti-DOTA C825 antigen binding fragment. For example, in some embodiments, the radiolabeled-DOTA hapten is administered 1 minute, 2 minutes, 3 minutes, 4 minutes, 5 minutes, 10 minutes, 15 minutes, 20 minutes, 25 minutes, 30 minutes, 35 minutes, 40 minutes, 45 minutes, 50 minutes, 55 minutes, 1 hour, 1.25 hours, 1.5 hours, 1.75 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 48 hours, 72 hours, 96 hours, or any range therein, following administration of the engineered immune cells expressing the anti-DOTA C825 antigen binding fragment. Alternatively, the radiolabeled-DOTA hapten may be administered at any time after 4 or more days following administration of the engineered immune cells expressing the anti-DOTA C825 antigen binding fragment.

In one aspect, the present disclosure provides a method for monitoring biodistribution of engineered immune cells in a subject comprising: (a) administering to the subject an effective amount of any engineered immune cell disclosed herein, wherein the engineered immune cell is configured to localize to a tissue expressing the target antigen recognized by the engineered immune cell; (b) administering to the subject an effective amount of a radiolabeled-DOTA hapten, wherein the radiolabeled-DOTA hapten is configured to bind to the anti-DOTA C825 antigen binding fragment expressed by the engineered immune cell; and (c) determining the biodistribution of engineered immune cells in the subject by detecting radioactive levels emitted by the radiolabeled-DOTA hapten that are higher than a reference value. In another aspect, the present disclosure provides a method for monitoring biodistribution of engineered immune cells in a subject comprising: (a) administering to the subject an effective amount of a complex comprising any engineered immune cell of the present technology and a radiolabeled DOTA hapten, wherein the complex is configured to localize to a tissue expressing the target antigen recognized by the engineered immune cell; and (b) determining the biodistribution of engineered immune cells in the subject by detecting radioactive levels emitted by the radiolabeled-DOTA hapten that are higher than a reference value.

In yet another aspect, the present disclosure provides a method for monitoring viability of engineered immune cells in a subject comprising: (a) administering to the subject an effective amount of any engineered immune cell disclosed herein, wherein the engineered immune cell is configured to localize to a tissue expressing the target antigen recognized by the engineered immune cell; (b) administering to the subject an effective amount of a radiolabeled-DOTA hapten, wherein the radiolabeled-DOTA hapten is configured to bind to the anti-DOTA C825 antigen binding fragment expressed by the engineered immune cell; (c) detecting radioactive levels emitted by the radiolabeled-DOTA hapten that are higher than a reference value at a first time point; (d) detecting radioactive levels emitted by the radiolabeled-DOTA hapten that are higher than a reference value at a second time point; and (e) determining that the engineered immune cells in the subject are viable when the radioactive levels emitted by the radiolabeled-DOTA hapten at the second time point are comparable to that observed at the first time point. In some embodiments, the method further comprises administering to the subject a second effective amount of the radiolabeled-DOTA hapten prior to step (d). Also disclosed herein is a method for monitoring viability of engineered immune cells in a subject comprising: (a) administering to the subject an effective amount of a complex comprising any engineered immune cell described herein and a radiolabeled DOTA hapten, wherein the complex is configured to localize to a tissue expressing the target antigen recognized by the engineered immune cell; (b) detecting radioactive levels emitted by the radiolabeled-DOTA hapten that are higher than a reference value at a first time point; (c) detecting radioactive levels emitted by the radiolabeled-DOTA hapten that are higher than a reference value at a second time point; and (d) determining that the engineered immune cells in the subject are viable when the radioactive levels emitted by the radiolabeled-DOTA hapten at the second time point are comparable to that observed at the first time point.

In yet another aspect, the present disclosure provides a method for monitoring expansion of engineered immune cells in a subject comprising: (a) administering to the subject an effective amount of any engineered immune cell described herein, wherein the engineered immune cell is configured to localize to a tissue expressing the target antigen recognized by the engineered immune cell; (b) administering to the subject a first effective amount of a radiolabeled-DOTA hapten, wherein the radiolabeled-DOTA hapten is configured to bind to the anti-DOTA C825 antigen binding fragment expressed by the engineered immune cell; (c) detecting radioactive levels emitted by the radiolabeled-DOTA hapten that are higher than a reference value at a first time point; (d) administering to the subject a second effective amount of the radiolabeled-DOTA hapten after step (c); (e) detecting radioactive levels emitted by the radiolabeled-DOTA hapten that are higher than a reference value at a second time point; and (f) determining that the engineered immune cells in the subject have expanded when the radioactive levels emitted by the radiolabeled-DOTA hapten at the second time point are higher relative to that observed at the first time point.

In any and all embodiments of the methods disclosed herein, the radioactive levels emitted by the complex or the radiolabeled-DOTA hapten are detected using positron emission tomography or single photon emission computed tomography. Additionally or alternatively, in any of the preceding embodiments of the methods disclosed herein, the engineered immune cell, the radiolabeled-DOTA hapten, or the complex is administered intratumorally, intravenously, intramuscularly, intraarterially, intrathecally, intracapsularly, intraorbitally, intradermally, intraperitoneally, transtracheally, subcutaneously, intracerebroventricularly, orally or intranasally. In some embodiments of the methods disclosed herein, the engineered immune cell(s), radiolabeled-DOTA haptens, or complexes are administered intravenously, intratumorally, intraperitoneally, subcutaneously, intramuscularly, or intratumorally.

In any and all embodiments of the methods disclosed herein, the subject has a cancer or tumor selected from among carcinoma, sarcoma, a melanoma, or a hematopoietic cancer. In some embodiments, the cancer or tumor is selected from among adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.

### Kits

The present technology provides kits containing components suitable for diagnosing cancer in a patient.

In one aspect, the kit comprises a composition including engineered immune cells comprising a tumor antigen-targeted receptor (e.g., a CAR) and an anti-DOTA C825 antigen binding fragment in unit dosage form. In particular embodiments, the cells further expresses at least one co-stimulatory ligand. In some embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, the engineered immune cell can be provided together with instructions for administering the engineered immune cell to a subject having or at risk of developing a neoplasia (e.g., solid tumor). In certain embodiments, the instructions include at least one of the following: description of the diagnostic agent; dosage schedule and administration for diagnosing or monitoring progression of a neoplasia (e.g., solid tumor) or symptoms thereof, precautions; warnings; indications; counter-indications; overdose information; adverse reactions; animal pharmacology; clinical studies; and/or references. The instructions may be printed directly on the container (when present), or as a label applied to the container, or as a separate sheet, pamphlet, card, or folder supplied in or with the container.

In another aspect, the kits of the present technology comprise a DOTA hapten (e.g., Bn-DOTA, NH₂-benzyl (Bn) DOTA, a bischelate of Formula II, or any of the DOTA haptens described herein etc.), at least one engineered immune cell of the present technology, and instructions for use. The kits may further comprise one or more radionuclides, such as ¹¹¹In, ⁶⁷Ga ⁵¹Cr, ⁵⁸Co, ^{99m}Tc, ^{103m}Rh, ^{195m}Pt, ¹¹⁹Sb, ¹⁶¹Ho, ^{189m}Os, ¹⁹²Ir, ²⁰¹Tl, ²⁰³Pb, ⁸⁹Zr, ⁶⁸Ga, or ⁶⁴Cu.

In some embodiments, the at least one engineered immune cell of the present technology binds to a tumor antigen target (e.g., BCMA, CD19, mesothelin, MUC16, PSCA, WT1, and PRAME). The at least one engineered immune cell of the present technology may be provided in the form of a prefilled syringe or autoinjection pen containing a sterile, liquid formulation or lyophilized preparation of the antibody (e.g., Kivitz et al., Clin. Ther. 28:1619-29 (2006)).

A device capable of delivering the kit components through an administrative route may be included. Examples of such devices include syringes (for parenteral administration) or inhalation devices.

The kit components may be packaged together or separated into two or more containers. In some embodiments, the containers may be vials that contain sterile, lyophilized formulations of a DOTA hapten and/or engineered immune cell composition that are suitable for reconstitution. A kit may also contain one or more buffers suitable for reconstitution and/or dilution of other reagents. Other containers that may be used include, but are not limited to, a pouch, tray, box, tube, or the like. Kit components may be packaged and maintained sterilely within the containers.

### EXAMPLES

### Example 1: Materials and Methods

CAR T cell transduction. FIGS. 3A-3C show three different strategies to virally transduce primary human T cells with both C825 and CD19-CAR. FIG. 3A show transduction with two single constructs, one encoding C825 with a GFP reporter (top) and one encoding the CD19 CAR (bottom). FIG. 3B shows a bicistronic construct encoding C825 with a transmembrane domain and GFP reporter and CD19 CAR, separated by P2A cleavage site. FIG. 3C shows a bicistronic construct encoding C825 with a Thy1 GPI linkage and His tag reporter and CD19 CAR, separated by a P2A cleavage site. Representative flow plots of transduction of primary human T cells are shown on the right.

### Example 2: In Vivo Tracking of the Engineered CAR T Cells of the Present Technology in Xenograft Models

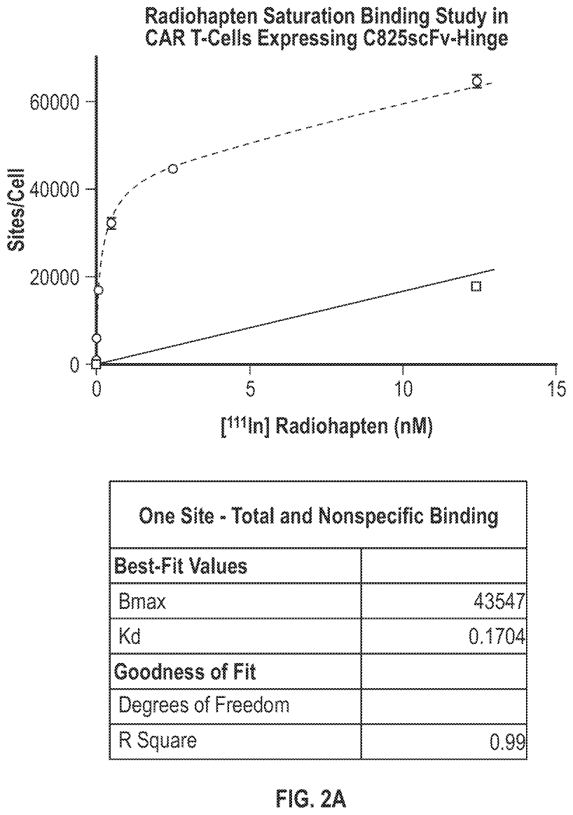

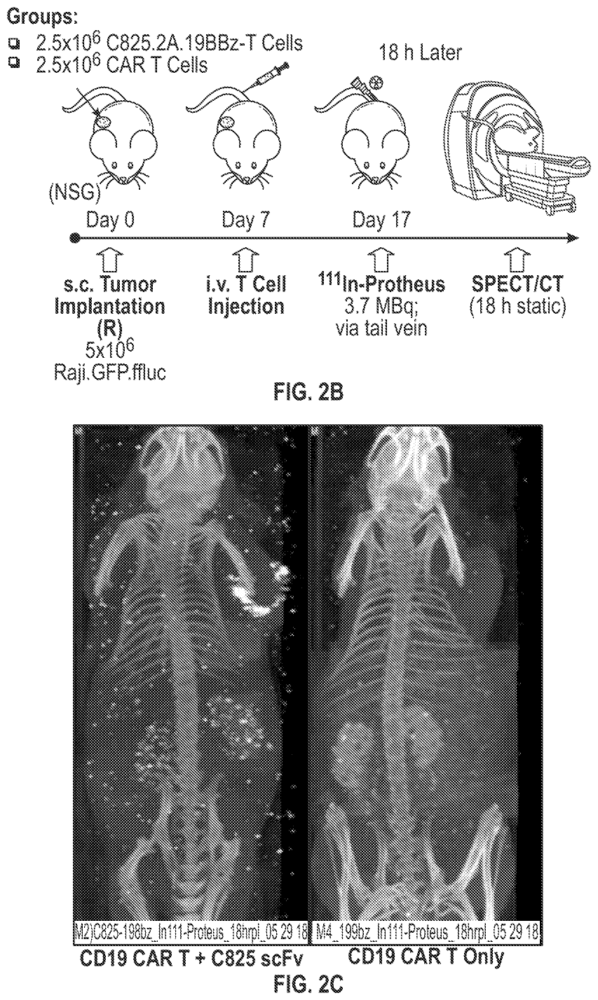

CD-19 CAR-T cells were transduced with a specialized ultra-high affinity membrane expressing hapten capture antibody C825. These cells were purified and tested for surface vector expression using [¹¹¹In]Pr-DOTA radiohapten system prior to in vivo use, by saturation binding assay as shown in FIG. 2A. CD19-expressing Raji lymphoma, a human B-cell (Burkitt's type) lymphoma, were used as a lymphomatous tumor in immunologically deficient mice for CD19 targeting. FIG. 2B shows a NSG mouse with a s.c. Raji GFP-fluc tumor in the right shoulder. Ten days after i.v. CAR-T cell injection (2.5×10⁶), the mouse was injected with [¹¹¹In]Pr-DOTA radiohapten for in vivo tracking of CAR T cells (either: CD19 CAR+C825, or control CD19 CAR only). As shown in FIG. 2C, animals treated with CD19 CAR T cells expressing C825 scFv showed effective tumor targeting in xenografts bearing Raji tumors. CAR-T cells efficiently captured radiohapten chelates with optimized pharmacology via renal clearance (data not shown).

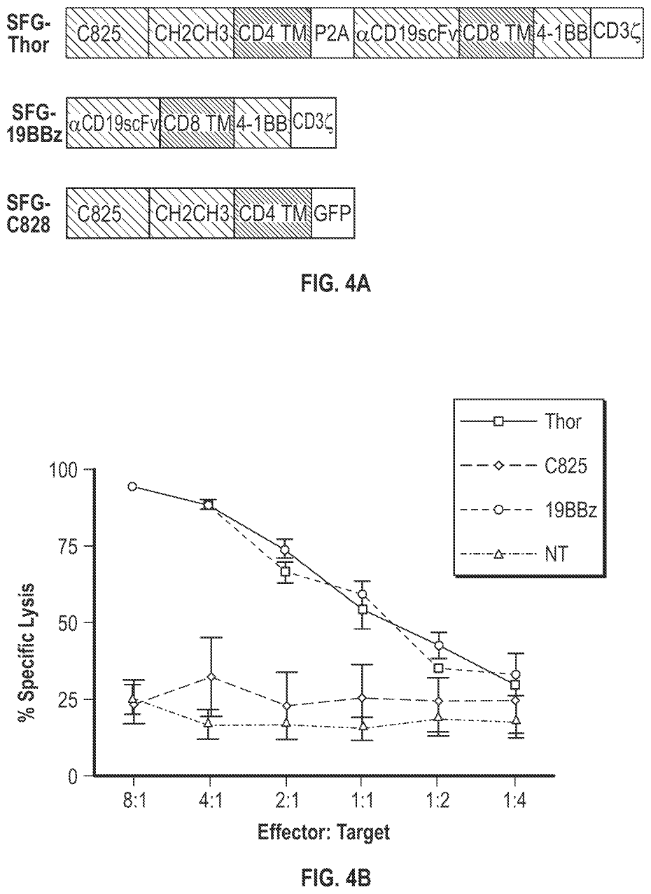

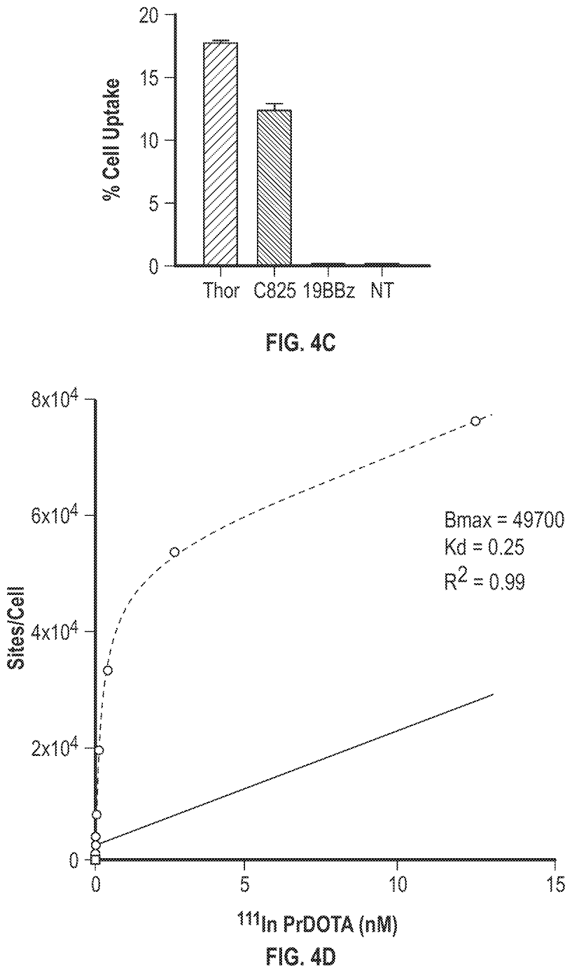

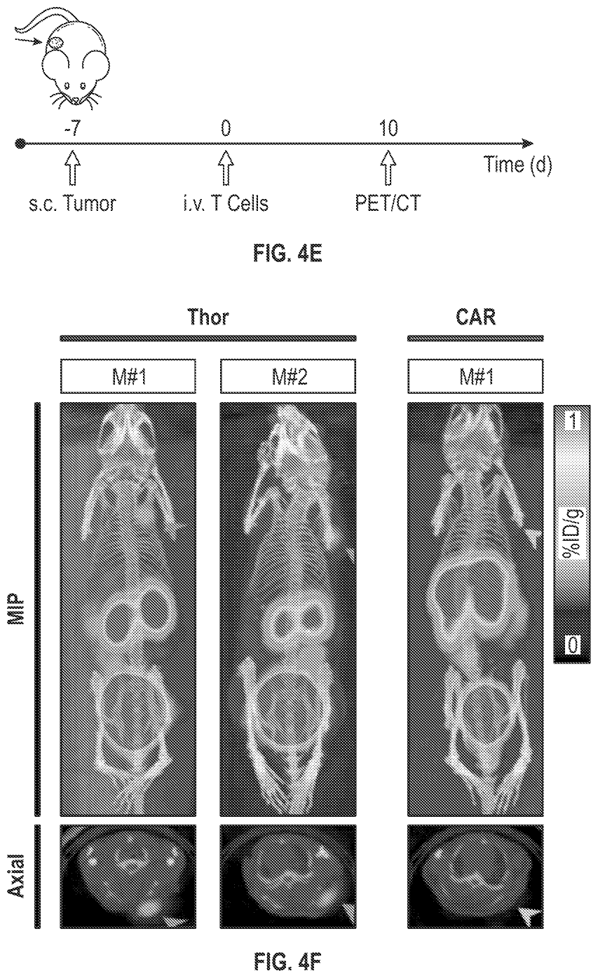

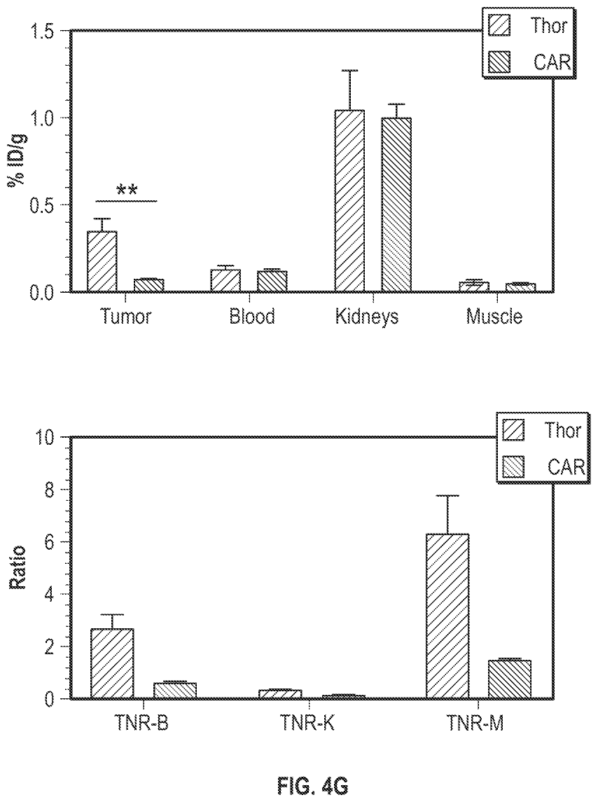

FIG. 4A shows schematic structures of retroviral vectors SFG-Thor, SFG-19BBz (CAR) and SFG-C825. FIG. 4B shows that there is no difference between SFG-Thor T cells and SFG-19BBz (CAR) T cells with respect to killing CD19(+) Raji tumor cells as measured by in vitro 4 h cytotoxicity assays. These results demonstrate that transducing CD19-specific CAR T cells with humanized C825 scFv did not negatively impact their ability to target and lyse CD19(+) tumor target cells. FIG. 4C shows in vitro binding of [¹¹¹In]InPr at 1 h. This representative data set demonstrates the specific binding of the radiolabeled DOTA probe to C825-expressing T cells, whereas no significant uptake was observed in SFG-19BBz (CAR) and NT T cells. (All experiments were performed in triplicate at 37° C.). Data are mean±SD. FIG. 4D shows in vitro binding kinetics of [¹¹¹In]InPr to SFG-Thor T cells (n=3 independent assays; representative example shown). FIG. 4E shows an exemplary scheme of in vivo study for assessing T cell targeting to tumor cells. ⁶⁸Ga-NODAGA-Pr (100mCu, 700 pmol) was used as the radiotracer and administered 10 days after T cell administration (1×10⁶ T cells) in NSG mice bearing CD19(+) Raji xenografts. FIG. 4F shows exemplary Maximum intensity projection (MIP) images at 1 h post-injection (p.i.) of ⁶⁸Ga-NODAGA-Pr depicting homing and accumulation of SFG-Thor T cells at the tumor (right shoulder, red arrow). No uptake above background at the tumor site is noted following SFG-19BBz (CAR) T cell administration (blue arrow). FIG. 4G shows mean uptake in tumors and tumor-to-normal-tissue-ratios (TNR) (SFG-Thor: n=4; SFG-19BBz (CAR): n=2) using image-based biodistribution. **, P<0.01.

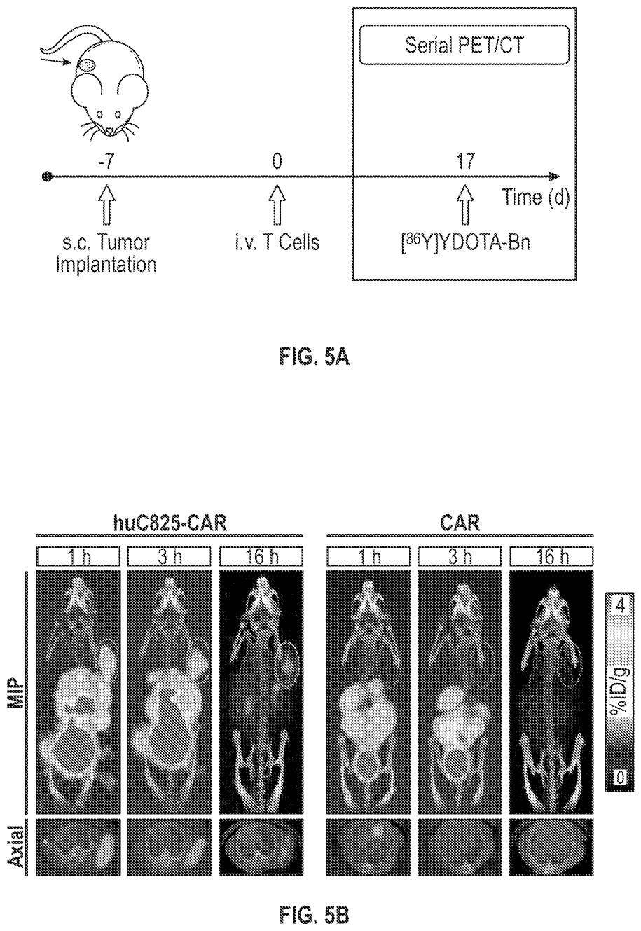

FIG. 5A shows an exemplary scheme for tracking engineered T cells in vivo in a s.c. Raji-tumor mouse model (3×10⁶ cells) with established treatment failure. Seven days post tumor inoculation, mice were injected i.v. with either 3×10⁶ huC825-19BBz or 3×10⁶ 19BBz T cells. On day 17 post T cell administration, mice demonstrating persistent growing tumor burden indicating treatment failure were i.v. injected with ⁸⁶Y-DOTA-Bn (3.7 MBq; 40 pmol) to assess persistence and localization of the transplanted T cells. FIG. 5B shows Maximum intensity projection (MIP) and axial PET/CT images at 1, 3 and 16 h p.i. depict accumulation of huC825-19BBz-CAR T cells at the tumor (orange circle). Highest intratumoral T cell uptake was seen at 3 h pi of 4.9% ID/g (vs 0.8% ID/g in control). No uptake above background at the tumor is noted in control mice (19BBz CAR; green circle). Rapid, predominant renal tracer clearance was noted.

These results demonstrate that the engineered immune cells of the present technology are useful in methods for determining the in vivo biodistribution, viability, and expansion of the engineered immune cells.

### EXEMPLARY EMBODIMENTS

The present disclosure may be described in terms of the following non-limiting embodiments:

Embodiment 1: The present application in one aspect provides an engineered immune cell comprising: (a) an anti-DOTA C825 antigen binding fragment comprising the amino acid sequence of any one of SEQ ID NOs: 35-39, 41 or 42, and/or a nucleic acid encoding the anti-DOTA C825 antigen binding fragment; and (b) a receptor that binds to a target antigen and/or a nucleic acid encoding the receptor.

Embodiment 2: The engineered immune cell of Embodiment 1, wherein the receptor is a T cell receptor.

Embodiment 3: The engineered immune cell of Embodiment 1 or 2, wherein the receptor is a native cell receptor.

Embodiment 4: The engineered immune cell of Embodiment 1 or 2, wherein the receptor is a non-native cell receptor.

Embodiment 5: The engineered immune cell of any one of Embodiments 1-4, wherein the receptor is a chimeric antigen receptor.

Embodiment 6: The engineered immune cell of Embodiment 5, wherein the nucleic acid encoding the anti-DOTA C825 antigen binding fragment comprises a leader sequence for secretion of the anti-DOTA C825 antigen binding fragment.

Embodiment 7: The engineered immune cell of any one of Embodiments 1-6, wherein the nucleic acid encoding the anti-DOTA C825 antigen binding fragment is operably linked to a promoter.

Embodiment 8: The engineered immune cell of Embodiment 7, wherein the promoter is a constitutive promoter.

Embodiment 9: The engineered immune cell of Embodiment 7, wherein the promoter is a conditional promoter.

Embodiment 10: The engineered immune cell of Embodiment 9, wherein the conditional promoter is induced by binding of the receptor to the target antigen.

Embodiment 11: The engineered immune cell of any one of Embodiments 1-10, wherein the target antigen is a tumor antigen.

Embodiment 12: The engineered immune cell of any one of Embodiments 1-11, wherein the nucleic acid encoding the receptor is operably linked to a constitutive promoter.

Embodiment 13: The engineered immune cell of any one of Embodiments 5-12, wherein the chimeric antigen receptor comprises (i) an extracellular antigen binding domain; (ii) a transmembrane domain; and (iii) an intracellular domain.

Embodiment 14: The engineered immune cell of Embodiment 13, wherein the extracellular antigen binding domain binds to the target antigen.

Embodiment 15: The engineered immune cell of any one of Embodiments 11-14, wherein the tumor antigen is selected from the group consisting of 5T4, alpha 5β1-integrin, 707-AP, A33, AFP, ART-4, B7H4, BAGE, Bcl-2, β-catenin, BCMA, Bcr-abl, MN/C IX antibody, CA125, CA19-9, CAMEL, CAP-1, CASP-8, CD4, CD5, CD19, CD20, CD21, CD22, CD25, CDC27/m, CD33, CD37, CD45, CD52, CD56, CD80, CD123, CDK4/m, CEA, c-Met, CS-1, CT, Cyp-B, cyclin B1, DAGE, DAM, EBNA, EGFR, ErbB3, ELF2M, EMMPRIN, EpCam, ephrinB2, estrogen receptor, ETV6-AML1, FAP, ferritin, folate-binding protein, GAGE, G250, GD-2, GM2, GnT-V, gp75, gp100 (Pmel 17), HAGE, HER-2/neu, HLA-A*0201-R170I, HPV E6, HPV E7, Ki-67, HSP70-2M, HST-2, hTERT (or hTRT), iCE, IGF-1R, IL-2R, IL-5, KIAA0205, LAGE, LDLR/FUT, LRP, MAGE, MART, MART-1/melan-A, MART-2/Ski, MC1R, mesothelin, MUC, MUC16, MUM-1-B, myc, MUM-2, MUM-3, NA88-A, NYESO-1, NY-Eso-B, p53, proteinase-3, p190 minor ber-abl, Pml/RARα, PRAME, progesterone receptor, PSA, PSCA, PSM, PSMA, ras, RAGE, RU1 or RU2, RORI, SART-1 or SART-3, survivin, TEL/AML1, TGFβ, TPI/m, TRP-1, TRP-2, TRP-2/INT2, tenascin, TSTA tyrosinase, VEGF, and WT1.

Embodiment 16: The engineered immune cell of any one of Embodiments 13-15, wherein the extracellular antigen binding domain comprises a single chain variable fragment (scFv).

Embodiment 17: The engineered immune cell of any one of Embodiments 13-16, wherein the extracellular antigen binding domain comprises a human scFv.

Embodiment 18: The engineered immune cell of any one of Embodiments 13-17, wherein the extracellular antigen binding domain comprises a CD19 scFv of SEQ ID NO: 3 or SEQ ID NO: 4.

Embodiment 19: The engineered immune cell of any one of Embodiments 13-18, wherein the extracellular antigen binding domain comprises a CD19 scFv having at least 80%, 85%, 90%, 95%, 96%, 97%, 98%, or 99% sequence identity to SEQ ID NO: 3 or SEQ ID NO: 4.

Embodiment 20: The engineered immune cell of any one of Embodiments 13-19, wherein the extracellular antigen binding domain comprises a signal peptide that is covalently joined to the N-terminus of the extracellular antigen binding domain.

Embodiment 21: The engineered immune cell of any one of Embodiments 13-20, wherein the transmembrane domain comprises a CD8 transmembrane domain.

Embodiment 22: The engineered immune cell of any one of Embodiments 13-21, wherein the intracellular domain comprises one or more costimulatory domains.

Embodiment 23: The engineered immune cell of Embodiment 22, wherein the one or more costimulatory domains are selected from a CD28 costimulatory domain, a CD3ζ-chain, a 4-1BBL costimulatory domain, or any combination thereof.

Embodiment 24: The engineered immune cell of any one of Embodiments 1-23, wherein the engineered immune cell is a lymphocyte.

Embodiment 25: The engineered immune cell of Embodiment 24, wherein the lymphocyte is a T cell, a B cell, or a natural killer (NK) cell.

Embodiment 26: The engineered immune cell of Embodiment 25, wherein the T cell is a CD4+ T cell or a CD8+ T cell.

Embodiment 27: The engineered immune cell of any one of Embodiments 1-26, wherein the engineered immune cell is a tumor infiltrating lymphocyte.

Embodiment 28: The engineered immune cell of any one of Embodiments 1-27, wherein the engineered immune cell is derived from an autologous donor or an allogenic donor.

Embodiment 29: A polypeptide comprising a chimeric antigen receptor and an anti-DOTA C825 antigen binding fragment comprising an amino acid sequence of any one of SEQ ID NOs: 35-39, 41 or 42.

Embodiment 30: The polypeptide of Embodiment 29, further comprising a self-cleaving peptide located between the anti-DOTA C825 antigen binding fragment and the chimeric antigen receptor.

Embodiment 31: The polypeptide of Embodiment 30, wherein the self-cleaving peptide is a P2A self-cleaving peptide.

Embodiment 32: The polypeptide of any one of Embodiments 29-31, wherein the anti-DOTA C825 antigen binding fragment comprises a leader sequence for secretion of the anti-DOTA C825 antigen binding fragment.

Embodiment 33: The polypeptide of any one of Embodiments 29-32, wherein the chimeric antigen receptor comprises (i) an extracellular antigen binding domain; (ii) a transmembrane domain; and (iii) an intracellular domain.

Embodiment 34: The polypeptide of Embodiment 33, wherein the extracellular antigen binding domain binds to a tumor antigen.

Embodiment 35: The polypeptide of Embodiment 34, wherein the tumor antigen is selected from among MUC16, mesothelin, CD19, WT1, PSCA, and BCMA.

Embodiment 36: The polypeptide of any one of Embodiments 33-35, wherein the extracellular antigen binding domain comprises a single chain variable fragment (scFv).

Embodiment 37: The polypeptide of any one of Embodiments 33-36, wherein the extracellular antigen binding domain comprises a CD19 scFv of SEQ ID NO: 3 or SEQ ID NO: 4.

Embodiment 38: The polypeptide of any one of Embodiments 33-37, wherein the extracellular antigen binding domain comprises a CD19 scFv having at least 80%, 85%, 90%, 95%, 96%, 97%, 98%, or 99% sequence identity to SEQ ID NO: 3 or SEQ ID NO: 4.

Embodiment 39: The polypeptide of any one of Embodiments 33-38, wherein the transmembrane domain comprises a CD8 transmembrane domain.

Embodiment 40: The polypeptide of any one of Embodiments 33-39, wherein the intracellular domain comprises one or more costimulatory domains.

Embodiment 41: The polypeptide of Embodiment 40, wherein the one or more costimulatory domains are selected from a CD28 costimulatory domain, a CD3ζ-chain, a 4-1BBL costimulatory domain, or any combination thereof.

Embodiment 42: A nucleic acid encoding the polypeptide of any one of Embodiments 29-41.

Embodiment 43: The nucleic acid of Embodiment 42, wherein the nucleic acid encoding the polypeptide is operably linked to a promoter.

Embodiment 44: The nucleic acid of Embodiment 43, wherein the promoter is a constitutive promoter.

Embodiment 45: The nucleic acid of Embodiment 43, wherein the promoter is a conditional promoter.

Embodiment 46: The nucleic acid of Embodiment 45, wherein the conditional promoter is inducible by the chimeric antigen receptor binding to an antigen.

Embodiment 47: A vector comprising the nucleic acid of any one of Embodiments 42-46.

Embodiment 48: The vector of Embodiment 47, wherein the vector is a viral vector or a plasmid.

Embodiment 49: The vector of Embodiment 47, wherein the vector is a retroviral vector.

Embodiment 50: A host cell comprising the nucleic acid of any one of Embodiments 42-46 or the vector of any one of Embodiments 47-49.

Embodiment 51: A complex comprising the engineered immune cell of any one of Embodiments 1-28 and a DOTA hapten, wherein the engineered immune cell is configured to bind to the DOTA hapten and a tumor antigen.

Embodiment 52: The complex of Embodiment 51, wherein the DOTA hapten is benzyl-DOTA, NH₂-benzyl (Bn) DOTA, DOTA-desferrioxamine, DOTA-Phe-Lys(HSG)-D-Tyr-Lys(HSG)-NH₂, Ac-Lys(HSG)D-Tyr-Lys(HSG)-Lys(Tscg-Cys)-NH₂, DOTA-D-Asp-D-Lys(HSG)-D-Asp-D-Lys(HSG)-NH₂; DOTA-D-Glu-D-Lys(HSG)-D-Glu-D-Lys(HSG)-NH₂, DOTA-D-Tyr-D-Lys(HSG)-D-Glu-D-Lys(HSG)-NH₂, DOTA-D-Ala-D-Lys(HSG)-D-Glu-D-Lys(HSG)-NH₂, DOTA-D-Phe-D-Lys(HSG)-D-Tyr-D-Lys(HSG)-NH₂, Ac-D-Phe-D-Lys(DOTA)-D-Tyr-D-Lys(DOTA)-NH₂, Ac-D-Phe-D-Lys(DTPA)-D-Tyr-D-Lys(DTPA)-NH₂, Ac-D-Phe-D-Lys(Bz-DTPA)-D-Tyr-D-Lys(Bz-DTPA)-NH₂, Ac-D-Lys(HSG)-D-Tyr-D-Lys(HSG)-D-Lys(Tscg-Cys)-NH₂, DOTA-D-Phe-D-Lys(HSG)-D-Tyr-D-Lys(HSG)-D-Lys(Tscg-Cys)-NH₂, (Tscg-Cys)-D-Phe-D-Lys(HSG)-D-Tyr-D-Lys(HSG)-D-Lys(DOTA)-NH₂, Tscg-D-Cys-D-Glu-D-Lys(HSG)-D-Glu-D-Lys(HSG)-NH₂, (Tscg-Cys)-D-Glu-D-Lys(HSG)-D-Glu-D-Lys(HSG)-NH₂, Ac-D-Cys-D-Lys(DOTA)-D-Tyr-D-Ala-D-Lys(DOTA)-D-Cys-NH₂, Ac-D-Cys-D-Lys(DTPA)-D-Tyr-D-Lys(DTPA)-NH₂, Ac-D-Lys(DTPA)-D-Tyr-D-Lys(DTPA)-D-Lys(Tscg-Cys)-NH₂, Ac-D-Lys(DOTA)-D-Tyr-D-Lys(DOTA)-D-Lys(Tscg-Cys)-NH₂, DOTA-RGD, DOTA-PEG-E(c(RGDyK))₂, DOTA-8-AOC-BBN, DOTA-PESIN, p-NO2-benzyl-DOTA, DOTA-biotin-sarcosine (DOTA-biotin), 1,4,7,10-tetraazacyclododecane-1,4,7,10-tetraacetic acid mono (N-hydroxysuccinimide ester) (DOTA-NHS), or DOTATyrLysDOTA.

Embodiment 53: The complex of Embodiment 51, wherein the DOTA hapten has the structure of Formula II

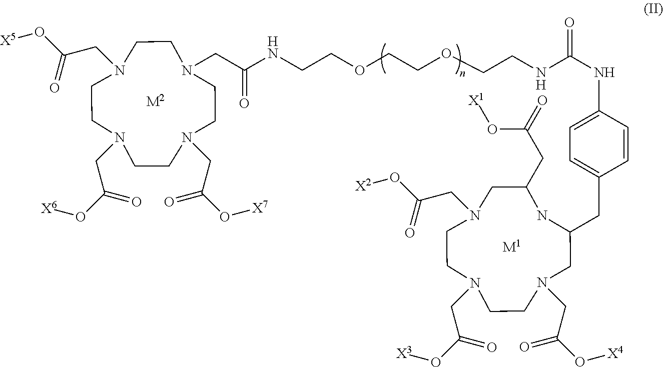

or a pharmaceutically acceptable salt thereof, wherein M¹ is ¹⁷⁵Lu³⁺, ⁴⁵Sc³⁺, ⁶⁹Ga³⁺, ⁷¹Ga³⁺, ⁸⁹Y³⁺, ¹¹³In³⁺, ¹¹⁵In³⁺, ¹³⁹La³⁺, ¹³⁶Ce³⁺, 138Ce³⁺, ¹⁴⁰Ce³⁺, ¹⁴²Ce³⁺, ¹⁵¹Eu³⁺, ¹⁵³Eu³⁺, ¹⁵⁹Tb³⁺, ¹⁵⁴Gd³⁺, ¹⁵⁵Gd³⁺, ¹⁵⁶Gd³⁺, ¹⁵⁷Gd³⁺, ¹⁵⁸Gd³⁺, or ¹⁶⁰Gd³⁺; M² is a radionuclide cation; X¹, X², X³, and X⁴ are each independently a lone pair of electrons (i.e., providing an oxygen anion) or H; X⁵, X⁶, and X⁷ are each independently a lone pair of electrons (i.e., providing an oxygen anion) or H; and n is 1, 2, 3, 4, 5, 6, 7, 8, 9, 10, 11, 12, 13, 14, 15, 16, 17, 18, 19, 20, 21, or 22.

Embodiment 54: The complex of any one of Embodiments 51-53, wherein M² is ¹¹¹In, ⁶⁷Ga ⁵¹Cr, ⁵⁸Co, ^{99m}Tc, ^{103m}Rh, ^{195m}Pt, ¹¹⁹Sb, ¹⁶¹Ho, ^{189m}Os, ¹⁹²Ir, ²⁰¹Tl, ²⁰³Pb, ⁸⁹Zr, ⁶⁸Ga, or ⁶⁴Cu.

Embodiment 55: A method for detecting tumors in a subject in need thereof comprising (a) administering to the subject an effective amount of the complex of Embodiment 54, wherein the complex is configured to localize to a tumor expressing the tumor antigen recognized by the engineered immune cell of the complex; and (b) detecting the presence of tumors in the subject by detecting radioactive levels emitted by the complex that are higher than a reference value.

Embodiment 56: A method for detecting tumors in a subject in need thereof comprising (a) administering to the subject an effective amount of the engineered immune cell of any one of Embodiments 11-28, wherein the engineered immune cell is configured to localize to a tumor expressing the tumor antigen recognized by the engineered immune cell; (b) administering to the subject an effective amount of a radiolabeled-DOTA hapten, wherein the radiolabeled-DOTA hapten is configured to bind to the anti-DOTA C825 antigen binding fragment expressed by the engineered immune cell; and (c) detecting the presence of tumors in the subject by detecting radioactive levels emitted by the radiolabeled-DOTA hapten that are higher than a reference value.

Embodiment 57: The method of Embodiment 55 or 56, wherein the radioactive levels emitted by the complex or the radiolabeled-DOTA hapten are detected using positron emission tomography or single photon emission computed tomography.

Embodiment 58: The method of any one of Embodiments 55-57, wherein the radioactive levels emitted by the complex or the radiolabeled-DOTA hapten are detected between 4 to 24 hours after the complex or the radiolabeled-DOTA hapten is administered.

Embodiment 59: The method of any one of Embodiments 55-58, wherein the radioactive levels emitted by the complex or the radiolabeled-DOTA hapten are expressed as the percentage injected dose per gram tissue (% ID/g).

Embodiment 60: The method of any one of Embodiments 55-59, wherein the ratio of radioactive levels between a tumor and normal tissue is about 2:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1 or 100:1.

Embodiment 61: The method of any one of Embodiments 55-60, wherein the subject is diagnosed with, or is suspected of having cancer.

Embodiment 62: The method of Embodiment 61, wherein the cancer is selected from the group consisting of adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.

Embodiment 63: The method of any one of Embodiments 55-62, wherein the complex, the engineered immune cell, or the radiolabeled-DOTA hapten is administered intravenously, intratumorally, intramuscularly, intraarterially, intrathecally, intracapsularly, intraorbitally, intradermally, intraperitoneally, transtracheally, subcutaneously, intracerebroventricularly, orally or intranasally.

Embodiment 64: The method of any one of Embodiments 55-63, wherein the complex, the engineered immune cell, or the radiolabeled-DOTA hapten is administered into the cerebral spinal fluid or blood of the subject.

Embodiment 65: A method for monitoring biodistribution of engineered immune cells in a subject comprising: (a) administering to the subject an effective amount of the engineered immune cell of any one of Embodiments 1-28, wherein the engineered immune cell is configured to localize to a tissue expressing the target antigen recognized by the engineered immune cell; (b) administering to the subject an effective amount of a radiolabeled-DOTA hapten, wherein the radiolabeled-DOTA hapten is configured to bind to the anti-DOTA C825 antigen binding fragment expressed by the engineered immune cell; and (c) determining the biodistribution of engineered immune cells in the subject by detecting radioactive levels emitted by the radiolabeled-DOTA hapten that are higher than a reference value.

Embodiment 66: A method for monitoring biodistribution of engineered immune cells in a subject comprising: (a) administering to the subject an effective amount of a complex comprising the engineered immune cell of any one of Embodiments 1-28 and a radiolabeled DOTA hapten, wherein the complex is configured to localize to a tissue expressing the target antigen recognized by the engineered immune cell; and (b) determining the biodistribution of engineered immune cells in the subject by detecting radioactive levels emitted by the radiolabeled-DOTA hapten that are higher than a reference value.

Embodiment 67: A method for monitoring viability of engineered immune cells in a subject comprising: (a) administering to the subject an effective amount of the engineered immune cell of any one of Embodiments 1-28, wherein the engineered immune cell is configured to localize to a tissue expressing the target antigen recognized by the engineered immune cell; (b) administering to the subject an effective amount of a radiolabeled-DOTA hapten, wherein the radiolabeled-DOTA hapten is configured to bind to the anti-DOTA C825 antigen binding fragment expressed by the engineered immune cell; (c) detecting radioactive levels emitted by the radiolabeled-DOTA hapten that are higher than a reference value at a first time point; (d) detecting radioactive levels emitted by the radiolabeled-DOTA hapten that are higher than a reference value at a second time point; and (e) determining that the engineered immune cells in the subject are viable when the radioactive levels emitted by the radiolabeled-DOTA hapten at the second time point are comparable to that observed at the first time point.

Embodiment 68: The method of Embodiment 67, further comprising administering to the subject a second effective amount of the radiolabeled-DOTA hapten prior to step (d).

Embodiment 69: A method for monitoring viability of engineered immune cells in a subject comprising: (a) administering to the subject an effective amount of a complex comprising the engineered immune cell of any one of Embodiments 1-28 and a radiolabeled DOTA hapten, wherein the complex is configured to localize to a tissue expressing the target antigen recognized by the engineered immune cell; (b) detecting radioactive levels emitted by the radiolabeled-DOTA hapten that are higher than a reference value at a first time point; (c) detecting radioactive levels emitted by the radiolabeled-DOTA hapten that are higher than a reference value at a second time point; and (d) determining that the engineered immune cells in the subject are viable when the radioactive levels emitted by the radiolabeled-DOTA hapten at the second time point are comparable to that observed at the first time point.

Embodiment 70: A method for monitoring expansion of engineered immune cells in a subject comprising: (a) administering to the subject an effective amount of the engineered immune cell of any one of Embodiments 1-28, wherein the engineered immune cell is configured to localize to a tissue expressing the target antigen recognized by the engineered immune cell; (b) administering to the subject a first effective amount of a radiolabeled-DOTA hapten, wherein the radiolabeled-DOTA hapten is configured to bind to the anti-DOTA C825 antigen binding fragment expressed by the engineered immune cell; (c) detecting radioactive levels emitted by the radiolabeled-DOTA hapten that are higher than a reference value at a first time point; (d) administering to the subject a second effective amount of the radiolabeled-DOTA hapten after step (c); (e) detecting radioactive levels emitted by the radiolabeled-DOTA hapten that are higher than a reference value at a second time point; and (f) determining that the engineered immune cells in the subject have expanded when the radioactive levels emitted by the radiolabeled-DOTA hapten at the second time point are higher relative to that observed at the first time point.

Embodiment 71: The method of any one of Embodiments 65-70, wherein the radioactive levels emitted by the complex or the radiolabeled-DOTA hapten are detected using positron emission tomography or single photon emission computed tomography.

Embodiment 72: The method of any one of Embodiments 65-71, wherein the engineered immune cell, the radiolabeled-DOTA hapten, or the complex is administered intravenously, intraperitoneally, subcutaneously, intramuscularly, or intratumorally.

Embodiment 73: The method of any one of Embodiments 65-72, wherein the cancer is a carcinoma, a sarcoma, a melanoma, or a hematopoietic cancer.

Embodiment 74: The method of any one of Embodiments 65-73, wherein the cancer is selected from among adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.

Embodiment 75: A kit comprising the engineered immune cell of any one of Embodiments 1-28, and instructions for diagnosing or monitoring the progression of cancer.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. An engineered immune cell comprising:
(a) an anti-DOTA C825 antigen binding fragment comprising the amino acid sequence of any one of SEQ ID NOs: 35-39, 41 or 42, and/or a nucleic acid encoding the anti-DOTA C825 antigen binding fragment; and
(b) a receptor that binds to a target antigen and/or a nucleic acid encoding the receptor, optionally wherein
the receptor is a T cell receptor, a native cell receptor, a non-native cell receptor, or a chimeric antigen receptor; or
the nucleic acid encoding the receptor is operably linked to a constitutive promoter; or
the engineered immune cell is derived from an autologous donor or an allogenic donor.

2. (canceled)

3. (canceled)

4. (canceled)

5. (canceled)

6. The engineered immune cell of claim 1, wherein the nucleic acid encoding the anti-DOTA C825 antigen binding fragment
comprises a leader sequence for secretion of the anti-DOTA C825 antigen binding fragment; or
is operably linked to a promoter, optionally wherein the promoter is a constitutive promoter or a conditional promoter, optionally wherein the conditional promoter is induced by binding of the receptor to the target antigen.

7. (canceled)

8. (canceled)

9. (canceled)

10. (canceled)

11. (canceled)

12. (canceled)

13. The engineered immune cell of claim 1, wherein the chimeric antigen receptor comprises
(i) an extracellular antigen binding domain, optionally wherein the extracellular antigen binding domain binds to the target antigen;
(ii) a transmembrane domain, optionally wherein the transmembrane domain comprises a CD8 transmembrane domain; and
(iii) an intracellular domain, optionally wherein the intracellular domain comprises one or more costimulatory domains, optionally wherein the one or more costimulatory domains are selected from a CD28 costimulatory domain, a CD3ζ-chain, a 4-1BBL costimulatory domain, or any combination thereof.

14. (canceled)

15. The engineered immune cell of claim 1, wherein the target antigen is a tumor antigen, optionally wherein the tumor antigen is selected from the group consisting of 5T4, alpha 5β1-integrin, 707-AP, A33, AFP, ART-4, B7H4, BAGE, Bcl-2, β-catenin, BCMA, Bcr-abl, MN/C IX antibody, CA125, CA19-9, CAMEL, CAP-1, CASP-8, CD4, CD5, CD19, CD20, CD21, CD22, CD25, CDC27/m, CD33, CD37, CD45, CD52, CD56, CD80, CD123, CDK4/m, CEA, c-Met, CS-1, CT, Cyp-B, cyclin B1, DAGE, DAM, EBNA, EGFR, ErbB3, ELF2M, EMMPRIN, EpCam, ephrinB2, estrogen receptor, ETV6-AML1, FAP, ferritin, folate-binding protein, GAGE, G250, GD-2, GM2, GnT-V, gp75, gp100 (Pmel 17), HAGE, HER-2/neu, HLA-A*0201-R170I, HPV E6, HPV E7, Ki-67, HSP70-2M, HST-2, hTERT (or hTRT), iCE, IGF-1R, IL-2R, IL-5, KIAA0205, LAGE, LDLR/FUT, LRP, MAGE, MART, MART-1/melan-A, MART-2/Ski, MC1R, mesothelin, MUC, MUC16, MUM-1-B, myc, MUM-2, MUM-3, NA88-A, NYESO-1, NY-Eso-B, p53, proteinase-3, p190 minor bcr-abl, Pml/RARα, PRAME, progesterone receptor, PSA, PSCA, PSM, PSMA, ras, RAGE, RU1 or RU2, RORI, SART-1 or SART-3, survivin, TEL/AML1, TGFβ, TPI/m, TRP-1, TRP-2, TRP-2/INT2, tenascin, TSTA tyrosinase, VEGF, and WT1.

16. The engineered immune cell of claim 13, wherein the extracellular antigen binding domain comprises
a single chain variable fragment (scFv), optionally wherein the extracellular antigen binding domain comprises a human scFv; or
a CD19 scFv of SEQ ID NO: 3 or SEQ ID NO: 4; or
a CD19 scFv having at least 80%, 85%, 90%, 95%, 96%, 97%, 98%, or 99% sequence identity to SEQ ID NO: 3 or SEQ ID NO: 4; or
a signal peptide that is covalently joined to the N-terminus of the extracellular antigen binding domain.

17. (canceled)

18. (canceled)

19. (canceled)

20. (canceled)

21. (canceled)

22. (canceled)

23. (canceled)

24. The engineered immune cell of claim 1, wherein the engineered immune cell is a lymphocyte, a T cell, a B cell, a natural killer (NK) cell, a CD4+ T cell, a CD8+ T cell, or a tumor infiltrating lymphocyte.

25. (canceled)

26. (canceled)

27. (canceled)

28. (canceled)

29. A polypeptide comprising a chimeric antigen receptor and an anti-DOTA C825 antigen binding fragment comprising an amino acid sequence of any one of SEQ ID NOs: 35-39, 41 or 42, optionally wherein the anti-DOTA C825 antigen binding fragment comprises a leader sequence for secretion of the anti-DOTA C825 antigen binding fragment.

30. The polypeptide of claim 29, further comprising a self-cleaving peptide located between the anti-DOTA C825 antigen binding fragment and the chimeric antigen receptor, optionally wherein the self-cleaving peptide is a P2A self-cleaving peptide.

31. (canceled)

32. (canceled)

33. The polypeptide of claim 29, wherein the chimeric antigen receptor comprises
(i) an extracellular antigen binding domain;
(ii) a transmembrane domain, optionally wherein the transmembrane domain comprises a CD8 transmembrane domain; and
(iii) an intracellular domain, optionally wherein the intracellular domain comprises one or more costimulatory domains, optionally wherein the one or more costimulatory domains are selected from a CD28 costimulatory domain, a CD3ζ-chain, a 4-1BBL costimulatory domain, or any combination thereof.

34. The polypeptide of claim 33, wherein the extracellular antigen binding domain
binds to a tumor antigen, optionally wherein the tumor antigen is selected from among MUC16, mesothelin, CD19, WT1, PSCA, and BCMA; or
comprises a single chain variable fragment (scFv); or
comprises a CD19 scFv of SEQ ID NO: 3 or SEQ ID NO: 4; or
comprises a CD19 scFv having at least 80%, 85%, 90%, 95%, 96%, 97%, 98%, or 99% sequence identity to SEQ ID NO: 3 or SEQ ID NO: 4.

35. (canceled)

36. (canceled)

37. (canceled)

38. (canceled)

39. (canceled)

40. (canceled)

41. (canceled)

42. A nucleic acid encoding the polypeptide of claim 29.

43. The nucleic acid of claim 42, wherein the nucleic acid encoding the polypeptide is operably linked to a promoter, optionally wherein the promoter is a constitutive promoter or a conditional promoter, optionally wherein the conditional promoter is inducible by the chimeric antigen receptor binding to an antigen.

44. (canceled)

45. (canceled)

46. (canceled)

47. A vector or host cell comprising the nucleic acid of claim 42, optionally wherein the vector is a viral vector, a retroviral vector or a plasmid.

48. (canceled)

49. (canceled)

50. (canceled)

51. A complex comprising the engineered immune cell of claim 1 and a DOTA hapten, wherein the engineered immune cell is configured to bind to the DOTA hapten and a tumor antigen, optionally wherein
the DOTA hapten is benzyl-DOTA, NH2-benzyl (Bn) DOTA, DOTA-desferrioxamine, DOTA-Phe-Lys(HSG)-D-Tyr-Lys(HSG)-NH2, Ac-Lys(HSG)D-Tyr-Lys(HSG)-Lys(Tscg-Cys)-NH2, DOTA-D-Asp-D-Lys(HSG)-D-Asp-D-Lys(HSG)-NH2; DOTA-D-Glu-D-Lys(HSG)-D-Glu-D-Lys(HSG)-NH2, DOTA-D-Tyr-D-Lys(HSG)-D-Glu-D-Lys(HSG)-NH2, DOTA-D-Ala-D-Lys(HSG)-D-Glu-D-Lys(HSG)-NH2, DOTA-D-Phe-D-Lys(HSG)-D-Tyr-D-Lys(HSG)-NH2, Ac-D-Phe-D-Lys(DOTA)-D-Tyr-D-Lys(DOTA)-NH2, Ac-D-Phe-D-Lys(DTPA)-D-Tyr-D-Lys(DTPA)-NH2, Ac-D-Phe-D-Lys(Bz-DTPA)-D-Tyr-D-Lys(Bz-DTPA)-NH2, Ac-D-Lys(HSG)-D-Tyr-D-Lys(HSG)-D-Lys(Tscg-Cys)-NH2, DOTA-D-Phe-D-Lys(HSG)-D-Tyr-D-Lys(HSG)-D-Lys(Tscg-Cys)-NH2, (Tscg-Cys)-D-Phe-D-Lys(HSG)-D-Tyr-D-Lys(HSG)-D-Lys(DOTA)-NH2, Tscg-D-Cys-D-Glu-D-Lys(HSG)-D-Glu-D-Lys(HSG)-NH2, (Tscg-Cys)-D-Glu-D-Lys(HSG)-D-Glu-D-Lys(HSG)-NH2, Ac-D-Cys-D-Lys(DOTA)-D-Tyr-D-Ala-D-Lys(DOTA)-D-Cys-NH2, Ac-D-Cys-D-Lys(DTPA)-D-Tyr-D-Lys(DTPA)-NH2, Ac-D-Lys(DTPA)-D-Tyr-D-Lys(DTPA)-D-Lys(Tscg-Cys)-NH2, Ac-D-Lys(DOTA)-D-Tyr-D-Lys(DOTA)-D-Lys(Tscg-Cys)-NH2, DOTA-RGD, DOTA-PEG-E(c(RGDyK))2, DOTA-8-AOC-BBN, DOTA-PESIN, p-NO2-benzyl-DOTA, DOTA-biotin-sarcosine (DOTA-biotin), 1,4,7,10-tetraazacyclododecane-1,4,7,10-tetraacetic acid mono (N-hydroxysuccinimide ester) (DOTA-NHS), or DOTATyrLysDOTA; or
wherein the DOTA hapten has the structure of Formula II 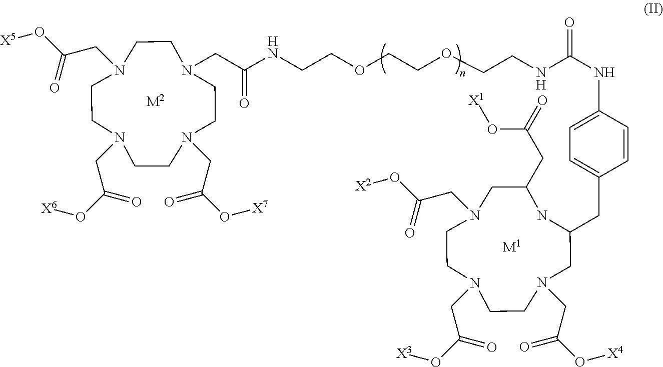
or a pharmaceutically acceptable salt thereof, wherein
M¹ is ¹⁷⁵Lu³⁺, ⁴⁵Sc³⁺, ⁶⁹Ga³⁺, ⁷¹Ga³⁺, ⁸⁹Y³⁺, ¹¹³In³⁺, ¹¹⁵In³⁺, ¹³⁹La³⁺, ¹³⁶Ce³⁺, 138Ce³⁺, ¹⁴⁰Ce³⁺, ¹⁴²Ce³⁺, ¹⁵¹Eu³⁺, ¹⁵³Eu³⁺, ¹⁵⁹Tb³⁺, ¹⁵⁴Gd³⁺, ¹⁵⁵Gd³⁺, ¹⁵⁶Gd³⁺, ¹⁵⁷Gd³⁺, ¹⁵⁸Gd³⁺, or ¹⁶⁰Gd³⁺;
M² is a radionuclide cation;
X¹, X², X³, and X⁴ are each independently a lone pair of electrons (i.e., providing an oxygen anion) or H;
X⁵, X⁶, and X⁷ are each independently a lone pair of electrons (i.e., providing an oxygen anion) or H; and
n is 1, 2, 3, 4, 5, 6, 7, 8, 9, 10, 11, 12, 13, 14, 15, 16, 17, 18, 19, 20, 21, or 22, optionally wherein M² is ¹¹¹In, ⁶⁷Ga, ⁵¹Cr, ⁵⁸Co, ^{99m}Tc, ^{103m}Rh, ^{195m}Pt, ¹¹⁹Sb, ¹⁶¹Ho, ^{189m}Os, ¹⁹²Ir, ²⁰¹Tl, ²⁰³Pb, ⁸⁹Zr, ⁶⁸Ga, or ⁶⁴Cu.

52. (canceled)

53. (canceled)

54. (canceled)

55. A method for detecting tumors in a subject in need thereof comprising
(I)
(a) administering to the subject an effective amount of a complex, wherein the complex is configured to localize to a tumor expressing a tumor antigen recognized by the engineered immune cell of the complex; and
(b) detecting the presence of tumors in the subject by detecting radioactive levels emitted by the complex that are higher than a reference value; or
(II)
(a) administering to the subject an effective amount of an engineered immune cell, wherein the engineered immune cell is configured to localize to a tumor expressing a tumor antigen recognized by the engineered immune cell;
(b) administering to the subject an effective amount of a radiolabeled-DOTA hapten, wherein the radiolabeled-DOTA hapten is configured to bind to an anti-DOTA C825 antigen binding fragment expressed by the engineered immune cell; and
(c) detecting the presence of tumors in the subject by detecting radioactive levels emitted by the radiolabeled-DOTA hapten that are higher than a reference value,
wherein the engineered immune cell is the engineered immune cell of claim 1, wherein the target antigen is a tumor antigen and wherein the complex comprises the engineered immune cell and a DOTA hapten.

56. (canceled)

57. The method of claim 55, wherein the radioactive levels emitted by the complex or the radiolabeled-DOTA hapten are detected using positron emission tomography or single photon emission computed tomography; or
wherein the radioactive levels emitted by the complex or the radiolabeled-DOTA hapten are detected between 4 to 24 hours after the complex or the radiolabeled-DOTA hapten is administered; or
wherein the radioactive levels emitted by the complex or the radiolabeled-DOTA hapten are expressed as the percentage injected dose per gram tissue (% ID/g); or
wherein the ratio of radioactive levels between a tumor and normal tissue is about 2:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1 or 100:1; or
wherein the subject is diagnosed with, or is suspected of having cancer, optionally wherein the cancer is selected from the group consisting of adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof; or
wherein the complex, the engineered immune cell, or the radiolabeled-DOTA hapten is administered intravenously, intratumorally, intramuscularly, intraarterially, intrathecally, intracapsularly, intraorbitally, intradermally, intraperitoneally, transtracheally, subcutaneously, intracerebroventricularly, orally or intranasally; or
wherein the complex, the engineered immune cell, or the radiolabeled-DOTA hapten is administered into the cerebral spinal fluid or blood of the subject.

58. (canceled)

59. (canceled)

60. (canceled)

61. (canceled)

62. (canceled)

63. (canceled)

64. (canceled)

65. A method for monitoring biodistribution of engineered immune cells in a subject comprising:
(I)
(a) administering to the subject an effective amount of an engineered immune cell, wherein the engineered immune cell is configured to localize to a tissue expressing a target antigen recognized by the engineered immune cell;
(b) administering to the subject an effective amount of a radiolabeled-DOTA hapten, wherein the radiolabeled-DOTA hapten is configured to bind to the anti-DOTA C825 antigen binding fragment expressed by the engineered immune cell; and
(c) determining the biodistribution of engineered immune cells in the subject by detecting radioactive levels emitted by the radiolabeled-DOTA hapten that are higher than a reference value; or
(II)
(a) administering to the subject an effective amount of a complex comprising an engineered immune cell and a radiolabeled DOTA hapten, wherein the complex is configured to localize to a tissue expressing a target antigen recognized by the engineered immune cell; and
(b) determining the biodistribution of engineered immune cells in the subject by detecting radioactive levels emitted by the radiolabeled-DOTA hapten that are higher than a reference value,
wherein the engineered immune cell is the engineered immune cell of claim 1.

66. (canceled)

67. A method for monitoring viability of engineered immune cells in a subject comprising:
(I)
(a) administering to the subject an effective amount of an engineered immune cell, wherein the engineered immune cell is configured to localize to a tissue expressing a target antigen recognized by the engineered immune cell;
(b) administering to the subject an effective amount of a radiolabeled-DOTA hapten, wherein the radiolabeled-DOTA hapten is configured to bind to the anti-DOTA C825 antigen binding fragment expressed by the engineered immune cell;
(c) detecting radioactive levels emitted by the radiolabeled-DOTA hapten that are higher than a reference value at a first time point;
(d) detecting radioactive levels emitted by the radiolabeled-DOTA hapten that are higher than a reference value at a second time point; and
(e) determining that the engineered immune cells in the subject are viable when the radioactive levels emitted by the radiolabeled-DOTA hapten at the second time point are comparable to that observed at the first time point, optionally wherein the method further comprises administering to the subject a second effective amount of the radiolabeled-DOTA hapten prior to step (d); or
(II)
(a) administering to the subject an effective amount of a complex comprising an engineered immune cell and a radiolabeled DOTA hapten, wherein the complex is configured to localize to a tissue expressing a target antigen recognized by the engineered immune cell;
(b) detecting radioactive levels emitted by the radiolabeled-DOTA hapten that are higher than a reference value at a first time point;
(c) detecting radioactive levels emitted by the radiolabeled-DOTA hapten that are higher than a reference value at a second time point; and
(d) determining that the engineered immune cells in the subject are viable when the radioactive levels emitted by the radiolabeled-DOTA hapten at the second time point are comparable to that observed at the first time point,
wherein the engineered immune cell is the engineered immune cell of claim 1.

68. (canceled)

69. (canceled)

70. A method for monitoring expansion of engineered immune cells in a subject comprising:
(a) administering to the subject an effective amount of the engineered immune cell of claim 1, wherein the engineered immune cell is configured to localize to a tissue expressing the target antigen recognized by the engineered immune cell;
(b) administering to the subject a first effective amount of a radiolabeled-DOTA hapten, wherein the radiolabeled-DOTA hapten is configured to bind to the anti-DOTA C825 antigen binding fragment expressed by the engineered immune cell;
(c) detecting radioactive levels emitted by the radiolabeled-DOTA hapten that are higher than a reference value at a first time point;
(d) administering to the subject a second effective amount of the radiolabeled-DOTA hapten after step (c);
(e) detecting radioactive levels emitted by the radiolabeled-DOTA hapten that are higher than a reference value at a second time point; and
(f) determining that the engineered immune cells in the subject have expanded when the radioactive levels emitted by the radiolabeled-DOTA hapten at the second time point are higher relative to that observed at the first time point.

71. The method of claim 65, wherein the radioactive levels emitted by the complex or the radiolabeled-DOTA hapten are detected using positron emission tomography or single photon emission computed tomography; or
wherein the engineered immune cell, the radiolabeled-DOTA hapten, or the complex is administered intravenously, intraperitoneally, subcutaneously, intramuscularly, or intratumorally; or
wherein the cancer is a carcinoma, a sarcoma, a melanoma, or a hematopoietic cancer; or
wherein the cancer is selected from among adrenal cancers, bladder cancers, blood cancers, bone cancers, brain cancers, breast cancers, carcinoma, cervical cancers, colon cancers, colorectal cancers, corpus uterine cancers, ear, nose and throat (ENT) cancers, endometrial cancers, esophageal cancers, gastrointestinal cancers, head and neck cancers, Hodgkin's disease, intestinal cancers, kidney cancers, larynx cancers, leukemias, liver cancers, lymph node cancers, lymphomas, lung cancers, melanomas, mesothelioma, myelomas, nasopharynx cancers, neuroblastomas, non-Hodgkin's lymphoma, oral cancers, ovarian cancers, pancreatic cancers, penile cancers, pharynx cancers, prostate cancers, rectal cancers, sarcoma, seminomas, skin cancers, stomach cancers, teratomas, testicular cancers, thyroid cancers, uterine cancers, vaginal cancers, vascular tumors, and metastases thereof.

72. (canceled)

73. (canceled)

74. (canceled)

75. (canceled)


SEQUENCE LISTING (43 sequences)

SEQ ID NO: 1 - protein, 15 aa, Artificial Sequence
  Description of Artificial Sequence Synthetic peptide
GGGGSGGGGSGGGGS

SEQ ID NO: 2 - DNA, 45 bp, Artificial Sequence
  Description of Artificial Sequence Synthetic oligonucleotide
ggcggcggcggatctggaggtggtggctcaggtggcggaggctcc

SEQ ID NO: 3 - protein, 245 aa, Homo sapiens
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 4 - protein, 263 aa, Artificial Sequence
  Description of Artificial Sequence Synthetic polypeptide
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 5 - DNA, 744 bp, Artificial Sequence
  Description of Artificial Sequence Synthetic polynucleotide
gaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgca

SEQ ID NO: 6 - DNA, 789 bp, Artificial Sequence
  Description of Artificial Sequence Synthetic polynucleotide
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgg

SEQ ID NO: 7 - protein, 21 aa, Artificial Sequence
  Description of Artificial Sequence Synthetic peptide
MALPVTALLLPLALLLHAARP

SEQ ID NO: 8 - DNA, 63 bp, Artificial Sequence
  Description of Artificial Sequence Synthetic oligonucleotide
atggccctgccagtaacggctctgctgctgccacttgctctgctcctccatgcagccaggcct

SEQ ID NO: 9 - protein, 18 aa, Artificial Sequence
  Description of Artificial Sequence Synthetic peptide
MALPVTALLLPLALLLHA

SEQ ID NO: 10 - DNA, 54 bp, Artificial Sequence
  Description of Artificial Sequence Synthetic oligonucleotide
atggctctcccagtgactgccctactgcttcccctagcgcttctcctgcatgca

SEQ ID NO: 11 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 12 - DNA, 321 bp, Homo sapiens
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 13 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 14 - protein, 71 aa, Homo sapiens
PTTTPAPRPPTPAPTIASQPLSLRPEACRPAAGGAVHTRGLDFACDIYIWAPLAGTCGVLLLSLVITLYCN

SEQ ID NO: 15 - DNA, 213 bp, Homo sapiens
cccaccacgacgccagcgccgcgaccaccaaccccggcgcccacgatcgcgtcgcagcccctgtccctgcgcccagaggcgtgccggccagcggcggggggcgcagtgcacacgagggggctggacttcgcctgtgatatctacatctgggcgcccctggccgggacttgtggggtccttctcctgtcactggttatcaccctttactgcaac

SEQ ID NO: 16 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 17 - protein, 164 aa, Homo sapiens; identical to SEQ ID NO: 16

SEQ ID NO: 18 - protein, 112 aa, Homo sapiens
RVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - DNA, 337 bp, Homo sapiens
agagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcg

SEQ ID NO: 21 - DNA, 339 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 22 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 23 - protein, 42 aa, Homo sapiens
KRGRKKLLYIFKQPFMRPVQTTQEEDGCSCRFPEEEEGGCEL

SEQ ID NO: 24 - DNA, 126 bp, Homo sapiens
aaacggggcagaaagaagctcctgtatatattcaaacaaccatttatgagaccagtacaaactactcaagaggaagatggctgtagctgccgatttccagaagaagaagaaggaggatgtgaactg

SEQ ID NO: 25 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 26 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 27 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 28 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 29 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 30 - protein, 370 aa, Homo sapiens
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 31 - protein, 289 aa, Homo sapiens
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 32 - protein, 10 aa, Homo sapiens
ALYVDSLFFL

SEQ ID NO: 33 - protein, 20 aa, Artificial Sequence
  Description of Artificial Sequence Synthetic peptide
GGGGSGGGGSGGGGSGGGGS

SEQ ID NO: 34 - protein, 30 aa, Artificial Sequence
  Description of Artificial Sequence Synthetic polypeptide
GGGGSGGGGSGGGGSGGGGSGGGGSGGGGS

SEQ ID NO: 35 - protein, 119 aa, Artificial Sequence
  Description of Artificial Sequence Synthetic polypeptide
HVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSS

SEQ ID NO: 36 - protein, 110 aa, Artificial Sequence
  Description of Artificial Sequence Synthetic polypeptide
QAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 37 - protein, 244 aa, Artificial Sequence
  Description of Artificial Sequence Synthetic polypeptide
HVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 38 - protein, 259 aa, Artificial Sequence
  Description of Artificial Sequence Synthetic polypeptide
HVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLG

SEQ ID NO: 39 - protein, 777 aa, Artificial Sequence
  Description of Artificial Sequence Synthetic polypeptide
TMNRGVPFRHLLLVLQLALLPAATQGHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGDLEPKSPDKTHTCPPCPAPPVAGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKAAAMALIVLGGVAGLLLFIGLGIFFCVRCRHMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 40 - protein, 491 aa, Artificial Sequence
  Description of Artificial Sequence Synthetic polypeptide
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 1053 aa, Artificial Sequence
  Description of Artificial Sequence Synthetic polypeptide
TMNRGVPFRHLLLVLQLALLPAATQGHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGKDPKAAAMALIVLGGVAGLLLFIGLGIFFCVRCRHMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKATNFSLLKQAGDVEENPGPALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 823 aa, Artificial Sequence
  Description of Artificial Sequence Synthetic polypeptide
TMNRGVPFRHLLLVLQLALLPAATQGHVQLVESGGGLVQPGGSLRLSCAASGFSLTDYGVHWVRQAPGKGLEWLGVIWSGGGTAYNTALISRFTISRDNSKNTLYLQMNSLRAEDTAVYYCARRGSYPYNYFDAWGCGTLVTVSSGGGGSGGGGSGGGGSQAVVTQEPSLTVSPGGTVTLTCGSSTGAVTASNYANWVQQKPGQCPRGLIGGHNNRPPGVPARFSGSLLGGKAALTLLGAQPEDEAEYYCALWYSDHWVIGGGTKLTVLGHHHHHHDKLVKCEGISLLAQNTSWLLLLLLSLSLLQATDFMSLATNFSLLKQAGDVEENPGPMALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 6 aa, Artificial Sequence
  Description of Artificial Sequence Synthetic 6xHis tag
HHHHHH